SEQUENCE LISTING (16 sequences)

SEQ ID NO: 1 - DNA, 31 bp, Artificial
  a NFAT responsive element
ggaggaaaaactgtttcatacagaaaggcgt

SEQ ID NO: 2 - DNA, 8 bp, Artificial
  a cAMP responsive element
cgacgtca

SEQ ID NO: 3 - protein, 19 aa, Artificial
  a self processing cleavage site
LLNFDLLKLAGDVESNPGP

SEQ ID NO: 4 - protein, 19 aa, Artificial
  a self processing cleavage site
TLNFDLLKLAGDVESNPGP

SEQ ID NO: 5 - protein, 13 aa, Artificial
  a self processing cleavage site
LKLAGDVESNPGP

SEQ ID NO: 6 - RNA, 25 nt, Artificial
  a siRNA sequence  (Chemically synthesized)
uaagcccaugcucugcuugaugcuc

SEQ ID NO: 7 - DNA, 15 bp, Artificial
  NFAT responsive element
ggaaaaactgtttca

SEQ ID NO: 8 - DNA, 8 bp, Artificial
  cAMP responsive element
tgacgtca

SEQ ID NO: 9 - DNA, 33 bp, Artificial
  a primer  (Chemically synthesized)
tatcatggatcctcagcaggactcctcaatcag

SEQ ID NO: 10 - DNA, 35 bp, Artificial
  a primer  (Chemically synthesized)
caagctggctagccaccatgtgcccaatgctactg

SEQ ID NO: 11 - DNA, 4591 bp, Artificial
  pcCBAD3 plasmid
attgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccagatccatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaggatcgatctgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagcgcgcgtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccttcgaaggtaagcctatccctaaccctctcctcggtctcgattctacgcgtaccggtcatcatcaccatcaccattgagtttaaacccgctgatcagcctcgaggcgccaacttgtttattgcagcttataatggttacaaataaagcaatagcatcacaaatttcacaaataaagcatttttttcactgcattctagttgtggtttgtccaaactcatcaatgtatcttatcatgtctggacccgtacg

SEQ ID NO: 12 - RNA, 25 nt, Artificial
  a siRNA sequence  (Chemically synthesized)
uucaaaggcacacacggcauccaug

SEQ ID NO: 13 - RNA, 25 nt, Artificial
  a siRNA sequence  (Chemically synthesized)
cauggaugccgugugugccuuugaa

SEQ ID NO: 14 - DNA, 6884 bp, Artificial
  “pcDNA5 TO G2A (mouse)” plasmid
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccatgagatcagaacctaccaatgcagcaggaaacaccacactgggggttacctccgttcttcagagcacctcagtaccttcttctgagacctgccacgtctcctacgaggagagcagagtggtcctggtggtggtgtacagtgccgtgtgcctgctgggcctaccagccaactgcctgactgcctggctgacgctgctgcaagtcctgcagaggaacgtgctagccgtctacctgttctgcctgtccctctgtgagctgctctacatcagcacggtgccattgtggatcatctacatccagaatcagcacaaatggaacctgggtccgcaggcctgcaaggtgactgcttacatcttcttctgcaacatctacatcagcatcctcttgctctgctgcatttcctgcgaccgctacatggccgtggtctatgcactggagagccgaggccaccgccaccagaggactgctgtcaccatttctgcgtgtgtgattcttcttgttggacttgttaactatccagtgtttgacatgaaggtggagaagagtttctgctttgagcccctgaggatgaacagcaagatagccggctaccactacctgcgtttcacctttggctttgccatccctctcggcatcctggcgttcaccaatcaccagatcttccggagcatcaaactcagtgacagcctgagcgctgcgcagaagaacaaggtgaagcgctccgccatcgcggtcgtcaccatcttcctggtctgctttgctccctaccacgtggtactcctcgtcaaagctgccagcttttccttctaccaaggagacatggatgccgtgtgtgcctttgaaagcagactgtacacagtctctatggtgtttctgtgcctgtctacagtcaacagtgtggctgaccccatcatctacgtgctgggtacagaccactctcggcaagaagtgtccagaatccacacagggtggaaaaagtggtccacaaagacatatgttacatgctcaaaggactctgaggagacacacttgcccacagagctttcaaacacatacaccttccccaatcccgcgcaccctccaggatcacagccagcgaagctaggtttactgtgctcgccagagagactgcctgaggagctctgctaagcggccgctcgagtctagagggcccttcgaaggtaagcctatccctaaccctctcctcggtctcgattctacgcgtaccggtcatcatcaccatcaccattgagtttaaacccgctgatcagcctcgaggcgccaacttgtttattgcagcttataatggttacaaataaagcaatagcatcacaaatttcacaaataaagcatttttttcactgcattctagttgtggtttgtccaaactcatcaatgtatcttatcatgtctggacccgtacgattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggttggttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - DNA, 1149 bp, Mouse
atgagatcagaacctaccaatgcagcaggaaacaccacactgggggttacctccgttcttcagagcacctcagtaccttcttctgagacctgccacgtctcctacgaggagagcagagtggtcctggtggtggtgtacagtgccgtgtgcctgctgggcctaccagccaactgcctgactgcctggctgacgctgctgcaagtcctgcagaggaacgtgctagccgtctacctgttctgcctgtccctctgtgagctgctctacatcagcacggtgccattgtggatcatctacatccagaatcagcacaaatggaacctgggtccgcaggcctgcaaggtgactgcttacatcttcttctgcaacatctacatcagcatcctcttgctctgctgcatttcctgcgaccgctacatggccgtggtctatgcactggagagccgaggccaccgccaccagaggactgctgtcaccatttctgcgtgtgtgattcttcttgttggacttgttaactatccagtgtttgacatgaaggtggagaagagtttctgctttgagcccctgaggatgaacagcaagatagccggctaccactacctgcgtttcacctttggctttgccatccctctcggcatcctggcgttcaccaatcaccagatcttccggagcatcaaactcagtgacagcctgagcgctgcgcagaagaacaaggtgaagcgctccgccatcgcggtcgtcaccatcttcctggtctgctttgctccctaccacgtggtactcctcgtcaaagctgccagcttttccttctaccaaggagacatggatgccgtgtgtgcctttgaaagcagactgtacacagtctctatggtgtttctgtgcctgtctacagtcaacagtgtggctgaccccatcatctacgtgctgggtacagaccactctcggcaagaagtgtccagaatccacacagggtggaaaaagtggtccacaaagacatatgttacatgctcaaaggactctgaggagacacacttgcccacagagctttcaaacacatacaccttccccaatcccgcgcaccctccaggatcacagccagcgaagctaggtttactgtgctcgccagagagactgcctgaggagctctgctaa

SEQ ID NO: 16 - DNA, 6565 bp, Artificial
  p4X-CRE-BLA-X plasmid
aattcccctcccgcgaaggcgtcggcgcggggctggcgtagggcctgcgtcagctgcagcccgccggagctcgacgtcacagtatgacggccatgggagctcaaattgacgtcatggtaaaaattgacgtcatggtaagaattcgaacacgcagatgcagtcggggcggcgcggtccgaggtccacttcgccatattaaggtgacgcgtgtggcctcgaacaccgagcgaccctgcaggtcgacctgcaggcatgcaagctttttgcagaagctcagaataaacgctcaactttccgccaccatggggcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcattgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactcgaaaaaaacccctgctcgacctgcaggcatgcaagctttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgccgcctgatgcggtattttctccttacgcatctgtgcggtatttcacaccgcatatggtgcactctcagtacaatctgctctgatgccgcatagttaagccagccccgacaccccgccaacacccgctgacgcgccctgacgggcttgtctgctcccggcatccgcttacagacaagctgtgaccgtctccgggagctgcatgtgtcagaggttttcacgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaagagcaactcggtcgccgccatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcatcgatatctagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagccggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacaggggaattcgagct

(19) United States
(12) Patent Application Publication
(10) Pub. No.: US 2009/0317858 A1
(43) Pub. Date: Dec. 24, 2009

(54) CELLULAR ASSAYS FOR SIGNALING RECEPTORS

(75) Inventor: Bonnie Jean Hanson, Madison, WI (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, 5791 Van Allen Way, CA (US)

(21) Appl. No.: 12/486,620

(22) Filed: Jun. 17, 2009

Related U.S. Application Data
(63) Continuation of application No. 11/672,841, filed on Feb. 8, 2007.
(60) Provisional application No. 60/771,011, filed on Feb. 8, 2006.

(51) Int. Cl. C12Q 1/02; C12N 5/00; C12P 21/06; C12N 15/00

(52) U.S. Cl. 435/29; 435/325; 435/69.1; 435/455

(57) ABSTRACT

The present invention provides cells and methods related to signaling receptors. The cells of the invention express the signaling receptors (e.g., in a constitutively active state). The cells are useful for analyzing the signaling receptors and their related pathways. The invention further provides methods for studying interactions of the signaling receptors and for small molecule screening, including high throughput methods. The invention further relates to expressing a signaling receptor (e.g., a GPCR) in a constitutively active state, even in the absence of the receptor's ligand. This allows for screening for inhibitors of the activated receptor's pathway without even knowing the ligand that activates the receptor, e.g., an orphan receptor. The invention further provides cell lines for expressing a signaling receptor in a constitutively active state. These cell lines are useful for high throughput screening assays of the invention.

### 1. CROSS REFERENCE TO RELATED APPLICATIONS

This application claims the benefit of U.S. Provisional Application No. 60/771,011, filed Feb. 8, 2006, the disclosure of which is incorporated herein by reference in its entirety.

### 2. FIELD OF THE INVENTION

The present invention provides cells and methods related to signaling receptors. The cells of the invention express the signaling receptors. The cells are useful for analyzing the signaling receptors and their related pathways. The invention further provides methods for studying interactions of the signaling receptors and for small molecule screening, including high throughput methods.

### 3. BACKGROUND OF THE INVENTION

In most instances, G-protein-coupled receptors (GPCRs) are seven transmembrane receptors, heptahelical receptors, or 7™ receptors. For the most part, GPCRs are a family of transmembrane receptors that transduce an extracellular signal (ligand binding) into an intracellular signal (G protein activation). The GPCRs are involved in numerous types of pathways including, but not limited to, intercellular communication, regulation of immune system pathways, autonomic nervous system transmission, and physiological senses (e.g., visual sense, sense of smell, behavioral and mood regulation). There are estimated to be five or six major classes of GPCRs. Examples of GPCRs include, but are not limited to, the class A or “rhodopsin-like” receptors; the class B or “secretin-like” receptors; the class C or “metabotropic glutamate-like” receptors; the Frizzled and Smoothened-related receptors; the adhesion receptor family or EGF-7™/LNB-7™ receptors; adiponectin receptors and related receptors; and chemosensory receptors including odorant, taste, vomeronasal and pheromone receptors. As examples, the GPCR superfamily in humans includes but is not limited to those receptor molecules described by Vassilatis, et al., Proc. Natl. Acad. Sci. USA, 100:4903-4908 (2003); Takeda, et al., FEBS Letters, 520:97-101 (2002); Fredricksson, et al., Mol. Pharmacol., 63:1256-1272 (2003); Glusman, et al., Genome Res., 11:685-702 (2001); and Zozulya, et al., Genome Biol., 2:0018.1-0018.12 (2001). Fredriksson, et al., (Mol. Pharmacol., 63:1256-1272 (2003)) describe five main GPCR families, named glutamate, rhodopsin, adhesion, frizzled/taste2, and secretin, forming the GRAFS classification system.

There are also a wide range of ligands recognized by GPCRs. Ligands include, but are not limited to, photons (e.g., rhodopsin) to small molecules (e.g., histamine receptors) to proteins (e.g., chemokine receptors). GPCRs are the target of about 40% of all prescription pharmaceuticals on the market. (Filmore, Modern Drug Discovery, November 2004, pp. 11)

A typical GPCR normally contains seven membrane-spanning regions, an extracellular N-terminus and an intracellular C-terminus. The extracellular domains of a GPCR receptor can be glycosylated. These extracellular loops typically contain two highly conserved cysteine residues for forming disulfide bonds to stabilize the receptor structure. Ligands of GPCRs typically bind within the transmembrane domain.

GPCRs are believed to exist in a conformational equilibrium between active and inactive states. (Rubenstein and Lanzara (1998) Journal of Molecular Structure 430: 57-71) The binding of ligands is thought to shift the equilibrium. Types of GPCR ligands include, but are not limited to: agonists which shift the equilibrium in favor of active states; inverse agonists which shift the equilibrium in favor of inactive states; and neutral antagonists which do not affect the equilibrium. When a GPCR in an active state encounters a G-protein, it may activate the G-protein. Additionally, binding of G-proteins to GPCRs can affect the GPCR's affinity for ligands. In some cases, evidence suggests some GPCRs may be able to signal without G-proteins.

Typically, GPCRs become less sensitive (e.g., desensitization) to their ligands when exposed to the ligands for a prolonged period of time. This downregulation can be caused by phosphorylation of the intracellular (or cytoplasmic) of a GPCR by a protein kinase. One mechanism involves cyclic AMP-dependent protein kinases (e.g., protein kinase A) are activated by a signal coming from the G protein, which was activated by the receptor, via adenylate cyclase and cAMP. In a feedback mechanism, these activated kinases phosphorylate the receptor. Typically, the longer the receptor remains active, the more kinases are activated and the more receptors are phosphorylated. Another mechanism involves G-protein-coupled receptor kinases (GRKs) which phosphorylate active GPCRs.

Phosphorylation of the receptor can cause translocation of the GPCR, wherein the GPCR is brought to the inside of the cell, where it is dephosphorylated and then brought back to the surface. One example of this mechanism is used to regulate long-term exposure, for example, to a hormone. Phosphorylation of the receptor can also cause arrestin linking. A phosphorylated GPCR is linked to arrestin molecules that prevent or inhibit the GPCR from binding and/or activating G proteins. One example of this mechanism is used with rhodopsin in retina cells to compensate for exposure to bright light. In some cases, arrestin binding to the receptor is a prerequisite for translocation.

Most GPCR-modulating drugs on the market were not initially targeted to a specific protein but were developed on the basis of functional activity observed in an assay. That they activated or inhibited a GPCR specifically was only later discovered. (Filmore, Modern Drug Discovery, November 2004, pp. 11) Currently, potential drugs are screened for modulating a specific protein (e.g., receptor) target(s). With regards to GPCRs, especially orphan-GPCRs, there is a need for assays to evaluate specific GPCR pathways and assays of screening various compounds for those that modulate activity of a specific GPCR(s).

Citation or discussion of a reference herein shall not be construed as an admission that such is prior art to the present invention.

### 4. SUMMARY OF THE INVENTION

The invention relates, in part, to assays for identifying modulators (e.g., agonists, inverse agonists, or antagonists) of signaling pathways, as well as compositions used in such assays. In some aspects, the invention involves the detection of an expression product which is transcribed in response to modulation of a signaling pathway. FIG. 1A shows embodiments of the invention which employ a cell that contains two nucleic acids (N.A.1 and N.A.2) which contain a promoter operatively linked to a coding region for a signaling pathway component (SPC) and a signaling pathway promoter operatively linked to a reporter, respectively. The signaling pathway component and the reporter may each independently be naturally resident in the cell or may be an added component. Thus, in some embodiments, the invention includes assays which function by contacting a cell with a potential agonist or antagonist of a signaling pathway followed by measuring a downstream activity of the signaling pathway. Examples of effects which can be measured include, but are not limited to, transcription of a particular cellular nucleic acid, translation of a particular gene and changes in concentrations of a compound(s) (e.g., calcium or cAMP).

Some embodiments of the invention provide, functional cell-based assays e.g., for high throughput screening or detection of small molecules that act as modulators of a cellular receptor's pathway (e.g., a GPCR's). Some embodiments of the invention provide coupled reactions wherein a signal from a cellular receptor (e.g., a GPCR) modulates a reporter gene/polypeptide system (e.g., a beta-lactamase system) and/or modulates the cellular concentration of a compound and wherein the change can be measured (e.g., calcium and/or cAMP levels). The invention provides various methods as described herein. For clarity, the invention can be used to screen for modulators of any component in the pathway. Using GPCRs as an example, the GPCR can be expressed in an active state in a cell of the invention as described herein. Potential modulators of the pathway can then be screened by methods of the invention described herein. Referring to FIG. 1B, a modulator of the pathway could act on as examples, the GPCR (e.g., be an agonist, inverse agonist, antagonist, or interfere with G-protein coupling), the G-protein (e.g., interfere with coupling to the GPCR or inhibit the G-protein's activation of another component of the reaction), component 1, component 2, or component 3.

For clarity, in some embodiments, the second promoter may be responsive to any step or component in the pathway. In other words, it does not have to be responsive to an end result of the pathway (e.g., calcium or cAMP level increase). Using FIG. 1B as an example, the step of the pathway involving activation of the GPCR, activation of the G-protein, component 1, component 2 or component 3 or combinations thereof can act on the second promoter. Of course if the desired result is to inhibit the end result or step of the pathway, one may want to more directly measure the end step (e.g., increase in cAMP or calcium levels).

Inter alia, the inventors have developed methods of constructing a stable cell line that is capable of expressing a SPC (e.g., a GPCR) in an activated state, e.g., wherein the SPC is toxic to the cell and/or inhibits the construction of a stable cell line when constitutively expressed. This embodiment of the invention provides a cell line that can be used to, inter alia, screen for compounds (e.g., small molecules) that modulate the activation state of a SPC (e.g., a GPCR or kinase) and/or modulate a pathway in which the SPC is involved. Some cell lines of the invention are particularly useful because, using a GPCR as an example; the GPCR can be expressed in an active state in the absence of a ligand. Many GPCRs are orphan receptors and their ligands are unknown. Methods and cells of the invention provide a method of assaying the activated functions of these orphan receptors without knowing their ligands. Although, the present invention is also useful for assaying the function of GPCRs whose ligands are known and provides the advantage that the ligand does not need to added for assays involving the activation state of the GPCR.

The description and embodiments provided herein are generally applicable to all signaling cellular receptors. In one embodiment, the cellular receptor is a GPCR. In some embodiments, a SPC is a GPCR, a kinase, a nuclear receptor, an ion channel or a G-protein. In one embodiment of the invention, the signaling pathway component is a GPCR.

The invention further provides related cells, nucleic acids and methods for constructing the cells of the invention.

One embodiment of the invention provides a cell comprising a nucleic acid comprising a regulatable promoter operatively linked to a G-protein-coupled receptor (GPCR) coding region. In some aspects of the invention, the regulatable promoter is selected from the group consisting of a tetracycline inducible promoter, a T-REx™ promoter, a heat shock inducible promoter, a heavy metal ion inducible promoter, or a nuclear hormone receptor inducible promoter or other promoter element whose activity is conditionally regulated. In one embodiment, the regulatable promoter comprises a tet operator.

In some embodiments, the GPCR is expressed in an active form. In some aspects of the invention, the GPCR is expressed in an active form in the absence of its ligand. In some embodiments, the GPCR is overexpressed in an active form in the absence of the GPCR's ligand.

In some embodiments, the regulatable promoter comprises a CMV promoter element. In some embodiments, the cell further comprises a selectable marker. In some embodiments, the selectable marker and GPCR coding region are on the same nucleic acid. In some aspects of the invention, the GPCR and selectable marker coding regions are operatively linked with an IRES or 2A-like sequence. In some embodiments, a GPCR and selectable marker coding regions are operatively linked to different promoters. In some aspects of the invention, the selectable marker and GPCR coding region are on different nucleic acids. In some aspects of the invention, the GPCR coding region is from a cDNA.

Embodiments of the invention include, but are not limited to, wherein the cell is selected from the group consisting of an animal cell, a plant cell, an insect cell, a yeast cell and a mammalian cell. In some embodiments, the cell is selected from the group consisting of a 293 cell, a HEK cell, a CHO cell, a Hela cell, a Freestyle™ 293F cell (Invitrogen, California), a Per.C6 cell, a COS cell, a Vero cell, a BHK cell, a mouse L cell, a Jurkat cell, a 153DG44 cell, a PC12 cells, a human T-lymphocyte cell, a Cos7 cell and a murine cell or derivatives of any of these cells. In one embodiment, the cell contains an intracellular calcium indicator.

In one embodiment, the nucleic acid is a DNA or RNA. In one embodiment, the nucleic acid is a viral vector. Viral vectors include, but are not limited to, those derived from a baculovirus, an adenovirus, an Adeno-associated virus, a lentivirus, a retrovirus, or other virus for delivery of genes into cells. In one embodiment, the nucleic acid is a plasmid. In some embodiments, the nucleic acid comprises a transposon. In some embodiments, the nucleic acid is a synthetic microchromosome.

In some aspects of the invention, the GPCR coding region codes for a Class A GPCR, a Class B GPCR, a Class C GPCR, a Class F/S GPCR, an orphan GPCR or a non-orphan GPCR. In some embodiments, the GPCR coding region codes for a G2A, mG2A or GPR23 GPCR. In some embodiments, the cell is engineered to express more than one GPCR. In some embodiments, the more than one GPCR is each expressed from a regulatable promoter. In some embodiments, the more than one GPCR is each expressed or operatively linked to the same regulatable promoter and is expressed on the same transcript.

In some embodiments, a cell further comprises a nucleic acid comprising a second promoter operatively linked to a coding region for a reporter polypeptide. In one embodiment, the regulatable promoter operatively linked to a GPCR coding region and the second promoter operatively linked to a coding region for a reporter polypeptide are on the same nucleic acid. In one embodiment, the regulatable promoter operatively linked to a GPCR coding region and the second promoter operatively linked to a coding region for a reporter polypeptide are on different nucleic acids. In some embodiments, the regulatable promoter is operatively linked to a GPCR coding region pre-existing in the genome of the cell. In some aspects of the invention, the second promoter is regulated directly or indirectly by the amount of activated GPCR. In one embodiment, the second promoter is regulated by the amount of or change in the amount of intracellular calcium. In some embodiments, the second promoter is regulated by the amount of or change in the amount of intracellular cAMP and/or calcium. In one embodiment, the second promoter comprises a calcium responsive element, a cAMP responsive element, an NFAT responsive element, a kinase C-responsive promoter or any combinations thereof. In some embodiments, an NFAT responsive element comprises the nucleotide sequence of SEQ ID NO:1. In some embodiments, a cAMP responsive element comprises the nucleotide sequence of SEQ ID NO:2.

In one embodiment, the second promoter (e.g., operatively linked to a reporter polypeptide region) is indirectly modulated by the activity of a promiscuous Gα15 protein, a chimeric G protein, a Gqi5, or a Gqo5. In some embodiments, the GPCR is coupled to either G-alpha-i, G-alpha-s or G-alpha-12 in the absence of a G-alpha-15 protein. In some embodiments, the GPCR is coupled to at least one G-protein selected from the group consisting of a Gi, a Go, a Gs, a Gq, a Ga12/13, a G-alpha15, a G-alpha16, a chimeric G proteins, a Gqi5, or a Gqo5.

In some embodiments of the invention, the reporter polypeptide is detectable directly or indirectly by fluorescence, light absorption, colorimetric readout, detecting an enzyme reaction, immunohistochemistry, immunofluorescence, flow cytometry, fluorescent-activated cell sorting (FACS), luminescence or FRET. In some aspects of the invention, the reporter polypeptide is selected from, but not limited to, the group consisting of a beta-lactamase (bla), a fluorescent polypeptide, a luciferase, a green fluorescent protein (GFP), a chloramphenicol acetyl transferase, an alkaline phosphatase a beta.-galactosidase, an alkaline phosphatase, and a human growth hormone. In some embodiments of the invention, expression of the reporter polypeptide is increased when the amount of activated GPCR is increased; is decreased when the amount of activated GPCR is increased; is increased when the amount of activated GPCR is decreased; or is decreased when the amount of activated GPCR is decreased.

In some embodiments, a cell of the invention does not contain a reporter polypeptide and/or coding region. Many GPCRs cause detectable changes in cellular levels of certain compounds, e.g., calcium and/or cAMP levels. One skilled in the art can readily detect these changes without a reporter polypeptide and/or coding region. For example, changes in calcium levels can be detected using Fluo-4 and changes in cAMP levels can be detected using a Lance assay (Perkin Elmer). Other methods for detecting cAMP and/or calcium levels are known in the art, some of which are described herein.

In some embodiments, the cell further comprises a nucleic acid encoding a polypeptide having a biological activity of a promiscuous G-alpha protein. In some aspects of the invention, the cell is stable. In other embodiments of the invention, the cell is not stable (e.g., transiently transfected).

In some embodiments, the cell further comprises and/or is contacted with a compound known to bind to the GPCR. In one embodiment, the cell further comprises a compound selected from the group consisting of phorbol ester, thapsigargin, ionomycin and a kinase inhibitor.

The present invention additionally provides various related methods. The cells of the invention can be utilized for various methods, e.g., related assays. One aspect of the invention provides, methods of expressing a GPCR from a cell comprising introducing into the cell a nucleic acid comprising a promoter operatively linked to a GPCR coding region. In some embodiments, the method comprises introducing the nucleic acid by transfection, electroporation, microinjection, or infection with a viral vector. In one embodiment, the promoter operatively linked to the GPCR coding region is a regulatable promoter.

Another embodiment of the invention provides methods of constructing a GPCR reporter cell comprising: (a) introducing into the cell a nucleic acid comprising a promoter operatively linked to a GPCR coding region and (b) introducing into the cell a nucleic acid comprising a second promoter operatively linked to a second coding region for a reporter polypeptide. In some embodiments, (a) is performed prior to (b); (b) is performed prior to (a); or (a) and (b) are performed essentially simultaneously. In some embodiments, the second promoter is regulated directly or indirectly by the amount of activated GPCR. In some embodiments, the second promoter regulates expression by the amount of or change in intracellular calcium and/or cAMP levels.

Some aspects of the invention provide methods of detecting or monitoring activity of a GPCR comprising: (a) culturing a cell of the invention under conditions wherein the GPCR is expressed; and (b) detecting the expression of the reporter polypeptide. Some methods of the invention further provide contacting the cell with a reporter polypeptide substrate.

Some aspects of the invention provide methods for measuring the ability of a compound(s) to affect or modulate activation of a GPCR comprising: (a) culturing a cell of the invention under conditions wherein the GPCR is expressed; (b) contacting the cell with the compound(s); and (c) measuring expression of the reporter polypeptide.

Some aspects of the invention provide methods of detecting or monitoring activity of a GPCR comprising: (a) culturing a cell comprising: (i) a nucleic acid comprising a regulatable promoter operatively linked to a G-protein-coupled receptor (GPCR) coding region; and (ii) a nucleic acid comprising a second promoter operatively linked to a coding region for a reporter polypeptide under conditions wherein the GPCR is expressed; and (b) detecting the expression of the reporter polypeptide.

Some aspects of the invention provide methods for measuring the ability of a compound(s) to affect or modulate activation of a GPCR comprising: (a) culturing a cell comprising; (i) a nucleic acid comprising a regulatable promoter operatively linked to a G-protein-coupled receptor (GPCR) coding region; and (ii) a nucleic acid comprising a second promoter operatively linked to a coding region for a reporter polypeptide under conditions wherein the GPCR is expressed; (b) contacting the cell with the compound; and (c) measuring expression of the reporter polypeptide. Some methods of the invention further comprise a second population of the cell of step (a) in the absence of the compound or in the presence of a different concentration of the compound and measuring expression of the reporter polypeptide in the second population of the cell. In some embodiments, the method further comprises measuring the expression of the reporter polypeptide before and after (b). In some aspects of the invention, the compound is determined to modulate activation of a GPCR if the measured expression in the presence and absence of the compound differ. In one embodiment, the measured expressions in the presence and absence of the second compound have a statistically significant difference.

Some aspects of the invention provide methods for determining whether activation of a cell pathway by a first compound activating a GPCR is capable of being modulated by a second compound comprising: (a) culturing a cell of the invention under conditions wherein the GPCR is expressed and contacting the cell with the first compound to form a first sample; (b) culturing a cell of the invention under conditions wherein the GPCR is expressed and contacting the cell with the first compound and second compound to form a second sample; and (c) measuring expression of the reporter polypeptide in the first and second samples.

Some aspects of the invention provide methods for determining whether activation of a cell pathway by a first compound activating a GPCR is capable of being modulated by a second compound comprising: (a) culturing a cell comprising: (i) a nucleic acid comprising a regulatable promoter operatively linked to a G-protein-coupled receptor (GPCR) coding region; and (ii) a nucleic acid comprising a second promoter operatively linked to a coding region for a reporter polypeptide under conditions wherein the GPCR is expressed and contacting the cell with the first compound to form a first sample; (b) culturing a cell comprising (i) a nucleic acid comprising a regulatable promoter operatively linked to a G-protein-coupled receptor (GPCR) coding region; and (ii) a nucleic acid comprising a second promoter operatively linked to a coding region for a reporter polypeptide under conditions wherein the GPCR is expressed and contacting the cell with the first compound and the second compound to form a second sample; and (c) measuring expression of the reporter polypeptide in the first and second samples.

In some embodiments, the second compound is determined to modulate activation of a cell pathway by a first compound If the measured expressions in the presence and absence of the second compound differ. In one embodiment, the second compound is determined to modulate activation of a cell pathway if the measured expressions in the presence and absence of the second compound are statistically significantly different. In some aspects of the invention, the culturing is in the presence of a factor that induces expression of the GPCR. In one embodiment, the factor is tetracycline, doxycycline or a heavy-metal. In one embodiment, the promoter of the GPCR is heat inducible. Methods of the invention can further comprise contacting the cell with a calcium increasing compound that increases calcium levels inside the cell; an ionomycin, a thapsigargin, or a phorbol myristate acetate or an analog thereof.

Other embodiments of the invention provide methods of identifying a GPCR for a ligand or of identifying a ligand for a GPCR, the method comprising: (a) expressing the GPCR in a cell of the invention; (b) contacting the cell with the ligand; and (c) detecting expression of a reporter polypeptide, wherein expression of the reporter polypeptide is regulated by the GPCR, e.g., by the state of activation of the GPCR.

Some embodiments of the invention provide methods of identifying a GPCR for a ligand or of identifying a ligand for a GPCR, the method comprising: (a) expressing the GPCR in a cell comprising (i) a nucleic acid comprising a regulatable promoter operatively linked to a G-protein-coupled receptor (GPCR) coding region; and (ii) a nucleic acid comprising a second promoter operatively linked to a coding region for a reporter polypeptide; (b) contacting the cell with the ligand; and (c) detecting expression of the reporter polypeptide, wherein expression of the reporter polypeptide is regulated by the GPCR.

Some embodiments of the invention provide kits comprising assay reagents and a container containing a cell or cells of the invention. In some embodiments, a kit of the invention further comprises a protocol for any methods of the invention. In some embodiments, a kit further comprises a compound known to interact with a GPCR(s) of interest.

### 5. BRIEF DESCRIPTION OF THE FIGURES

For the purpose of illustrating the invention, there are depicted in the drawings certain embodiments on the invention. However, the invention is not limited to the precise arrangements and instrumentalities of the embodiments depicted in the drawings.

FIG. 1A shows a cell (represented by a circle) which contains two nucleic acids (N.A.1 and N.A.2). These nucleic acids may be part of the same nucleic acid molecule or on different nucleic acid molecules. One of these nucleic acids (N.A.1), is composed of a promoter (P) and a coding region for a signaling pathway component (SPC). The other nucleic acid (N.A.2) is composed of a signaling pathway promoter (SPP) and a reporter coding sequence (e.g., a nucleic acid which encodes beta-lactamase, beta-galactosidase, etc.).

FIG. 1B depicts some embodiments of the invention using a GPCR pathway as an example of a receptor for a signaling pathway. The first construct comprising the regulatable promoter and GPCR coding region may be on the same or a different nucleic acid as the second construct comprised of the second promoter and reporter coding region. The figure depicts that signaling pathway component 3 causes directly or indirectly the increased or decreased transcription from the second promoter. This is just shown as an example. The invention contemplates that any signaling pathway component can activate the second promoter (e.g., 1, 2, 3, the G-protein or the GPCR. Additionally, the compound(s) may act upon any component of the pathway or even multiple components of the pathway.

FIG. 2 depicts a schematic diagram illustrating the mechanism of action of the T-REx™ System. 1. Tet repressor (tetR) protein is expressed from pcDNA6/TR© in cultured cells. 2. TetR homodimers bind to Tet operator 2 (TetO₂) sequences in the inducible expression vector, repressing transcription of the gene of interest. 3. Upon addition, tetracycline (tet) binds to tetR homodimers. 4. Binding of tet to tetR homodimers causes conformational change in tetR, release from the Tet operator sequences, and induction of transcription from the gene of interest.

FIG. 3 depicts a map of the pcDNA5 G2A/TO expression plasmid used in construction of the T-REx™-G2A-NFAT-bla Freestyle™293F assay.

FIG. 4 depicts a map of the pcDNA6/TR expression plasmid used in construction of the T-REx™-G2A-NFAT-bla Freestyle™293F assay. pcDNA6/TR© is 6662 nucleotides and comprises a CMV promoter (bases 232-819); a Rabbit β-globin intron II (IVS) (bases 1028-1600); TetR gene (bases 1684-2340); SV40 early polyadenylation sequence (bases 2346-2477); f1 origin (bases 2897-3325); SV40 promoter and origin (bases 3335-3675); EM-7 promoter (bases 3715-3781); Blasticidin resistance gene (bases 3782-4180); SV40 early polyadenylation sequence (bases 4338-4468); pUC origin (bases 4851-5521); bla promoter (complementary strand) (bases 6521-6625); and Ampicillin (bla) resistance gene (complementary strand) (bases 5666-6526)

FIG. 5 shows parental cell lines transiently transfected with a G2A expression plasmid.

FIG. 6 shows beta-lactamase expression of TR CRE-bla Freestyle™ cells transiently transfected with a G2A coding region in a Tet inducible promoter construct and stimulated with various amounts of tetracycline for 24 h.

FIG. 7 shows beta-lactamase expression of TR NFAT-bla Freestyle™ cells transiently transfected with a G2A coding region in a Tet inducible promoter construct and stimulated with various amounts of tetracycline for 24 h.

FIG. 8 shows beta-lactamase expression from a T-REx™ G2A CRE-bla Freestyle™ 293F stimulation time experiment.

FIG. 9 shows beta-lactamase expression from a T-REx™ G2A NFAT-bla Freestyle™ 293F stimulation time experiment.

FIG. 10 shows dose response curves generated from cells stimulated with a dilution series of tetracycline starting at 100 ng/mL with 1:10 dilutions using 16 h tetracycline stimulation in Poly-D-Lysine coated plates.

FIG. 11 shows dose response curves generated from cells stimulated with a dilution series of tetracycline or doxycycline starting at 10 ug/mL with 1:10 dilutions.

FIG. 12 shows results from RNAi experiments. 12A) Clone #20 12B) Clone #40 12C) Clone #46.

FIG. 13 shows a dose response curve generated from cells stimulated for 16 hours with a dilution series of doxycycline starting at 100 ng/mL with 1:10 dilutions.

FIG. 14 shows dose response curves generated from G2A clone #20 cells stimulated with a dilution series of doxycycline starting at 100 ng/mL with 1:5 dilutions.

FIG. 15 shows dose response curves generated from G2A clone #20 cells stimulated with a dilution series of the doxycycline starting at 100 ng/mL with 1:5 dilutions in varying DMSO concentrations.

FIG. 16 shows dose response curves generated from cells stimulated with a dilution series of the doxycycline starting at 100 ng/mL with 1:5 dilutions. Cells were loaded with LiveBLAzer™-FRET B/G substrate for 60, 90, or 120 minutes.

FIG. 17 shows dose response curves generated from cells stimulated with a dilution series of the doxycycline starting at 20 ng/mL with 1:3 dilutions run on 3 separate days.

FIG. 18 shows dose response curves generated from freshly thawed cells stimulated with a dilution series of doxycycline starting at 20 ng/mL with 1:3 dilutions.

FIG. 19 shows T-REx™-NFAT-bla Freestyle™/293F cells stimulated for 16 hours with doxycycline in the presence of 0.5% DMSO. Cells were then loaded with LiveBLAzer™-FRET B/G (CCF4-AM) for 2 hours. Fluorescence emission values at 460 nm and 530 nm are obtained using a standard fluorescence plate reader and the Blue/Green Emission ratios are plotted against the concentration of the stimulant.

FIG. 20 is an example for a diagram of a process flow for cell line development using e.g., FACS.

FIG. 21 shows a map of the vector pcCBAD3.

FIG. 22 is a map of the pcDNA5 mG2A/TO expression plasmid used in construction of the TREx™-mG2A-NFAT-bla Freestyle293F cell lines and related assays.

FIG. 23 shows transient transfection data for the TR NFAT-bla cell line transfected with an mG2A expression plasmid.

FIG. 24 shows doxycycline dose response curves obtained for both the green and the turquoise sorted pools of stable T-REx mG2A NFAT-bla Freestyle 293F cell pools.

FIG. 25 shows blue/green ratios of six T-REx mG2A NFAT-bla Freestyle 293F clones selected from the initial round of sorting.

FIG. 26 shows a vector map of the plasmid p4X-CRE-BLA-X.

FIG. 27 is an exemplary flow chart showing a process for producing cells of the invention.

FIG. 28 shows RNAi verification to confirm that the observed increase in beta-lactamase blue:green ratios was due to mG2A expression. The MedGC is a negative control siRNA made up of a random medium GC rich sequence. The BLA is a positive control consisting of siRNA directed towards beta-lactamase. The siRNA #1 is directed towards mG2A. FIGS. 28A, 28B and 28 C show results for clone #2, #25 and #53, respectively.

FIG. 29 shows TREx™-mG2A-NFAT-bla Freestyle293F cells doxycycline response in the presence of 0.5% DMSO. The results produced an EC₅₀ for clone #2 of 386 pg/ml; for clone #25 of 1.12 ng/ml; for and clone #53 of 524 pg/ml.

FIG. 30 shows results from a transient transfection assay of GPR23 into CellSensor™ cell lines.

FIG. 31 shows an LPA dose response on the hGPR23-CRE-bla CHO-K1 selected pool and CRE-bla CHO-K1 cell lines with a resulting EC₅₀ of 258 nM for the hGPR23 CRE-bla CHO cells and of 239 nM for the CRE-bla CHO cells.

FIG. 32 shows results of a Perkin Elmer LANCE cAMP assay run on two inducible T-REx™-GPR23-CHO-K1 clones and a parental control. The LPA EC₅₀ results were: parent=5 μM; parent induced=915 nM; E1 clone=2 μM; E1 clone induced=30.3 nM; H6 clone=1.5 μM; and H6 induced=13.8 nM.

FIG. 33 shows tetracycline induced versus uninduced for six T-REx™-GPR23-CRE-bla-CHO-K1 clones to evaluate their inducible GPR23 specific activity. Clone H6-E2 gave the greatest inducible response (about 9.2 fold) and was chosen as a clone for an inverse agonist assay for GPR23.

FIG. 34 shows results for cell density experiments at different doxycycline concentrations using T-REx™-GPR23-CRE-bla-CHO-K1 clone H6-E2. The assay performed the best plating 20,000 cells per well with a maximum response ratio of 5.7 fold and a Z′ value of 0.8. The assay could also be run at 10,000 or 5,000 cells per well with only a small effect on the assay window. The EC₅₀ values for doxycycline were 1.3 ng/ml, 1.0 ng/ml, 1.6 ng/ml and 2.0 ng/ml for 2,500, 5,000, 10,000, and 20,000 cells/well, respectively.

FIG. 35 shows results for different induction times with doxycycline using T-REx™-GPR23-CRE-bla-CHO-K1 clone H6-E2. The widest assay window was achieved with a 24 hour (hr) induction time. The EC₅₀ values for doxycycline were 4.0 ng/ml, 1.9 ng/ml and 2.0 ng/ml for 16, 20 and 24 hours respectively.

FIG. 36 shows results for different GeneBLAzer® substrate loading times using T-REx™-GPR23-CRE-bla-CHO-K1 clone H6-E2. The cells were then loaded with LiveBLAzer™-FRET B/G substrate (2 μM) containing solution D for 1, 1.5 or 2 hours (hrs). The widest assay window was achieved with a 2 hr substrate loading time.

FIG. 37 shows results to analyze assay reproducibility.

FIG. 38 shows results comparing freshly thawed T-REx™-GPR23-CRE-bla-CHO-K1 cells to passaged cells. There was no significant change in the assay window or the Z′ values of the assay when it was run using recently thawed cells.

FIG. 39 shows LPA responsiveness of the T-REx™-GPR23 CRE-bla-CHO-K1 Clone H6-E2. The induced T-REx™-GPR23-CRE-bla-CHO-K1 Clone H6-E2 cells showed a shifted EC₅₀ of LPA to 2.3 nM from the 628 μM of the un-induced cells. The response of the cells to LPA decreases from 9 fold in the un-induced cells to 2.3 fold in the induced cells due to the constitutive activity of the receptor.

FIG. 40 shows a dose response of T-REx-GPR23-CRE-bla CHO-K1 cells to doxycycline. Blue/Green Emission Ratios were plotted against the indicated concentrations of doxycycline.

### 6. BRIEF DESCRIPTION OF THE SEQUENCES

SEQ ID NO:1 an NFAT responsive element:

GGAGGAAAAACTGTTTCATACAGAAAGGCGT.

SEQ ID NO:2 a cAMP responsive element:

CGACGTCA.

SEQ ID NO:3-5 are examples of self processing cleavage sites:

LLNFDLLKLAGDVESNPGP (SEQ ID NO: 3);
TLNFDLLKLAGDVESNPGP (SEQ ID NO: 4);
and
LKLAGDVESNPGP (SEQ ID NO: 5).

SEQ ID NO:6 is an example of an siRNA sequence:

| | |
|---|---|
| UAAGCCCAUGCUCUGCUUGAUGCUC. | (SEQ ID NO: 6) |

SEQ ID NO:7 is an NFAT responsive element (e.g., fragment of SEQ ID NO: 1):

GGAAAAACTGTTTCA.

SEQ ID NO:8 is a cAMP responsive element:

TGACGTCA.

SEQ ID NO:9 and 10 are primers:

(SEQ ID NO: 9)

G2arevbamHI-TATCATGGATCCTCAGCAGGACTCCTCAATCAG
and

(SEQ ID NO: 10)

G2aforNHE-CAAGCTGGCTAGCCACCATGTGCCCAATGCTACTG

SEQ ID NO:11 is the sequence of the vector pcCBAD3.

SEQ ID NO:12 is the “upper stand” of siRNA #1:

(SEQ ID NO: 12)

5′ to 3′ UUC AAA GGC ACA CAC GGC AUC CAU G.

SEQ ID NO:13 is the “lower stand” of siRNA #1:

(SEQ ID NO: 13)

5′ to 3′ CAU GGA UGC CGU GUG UGC CUU UGA A.

SEQ ID NO:14 is the nucleotide sequence of the “pcDNA5 TO G2A (mouse)” plasmid.

SEQ ID NO:15 is the nucleotide sequence of a mG2a coding region:

ATGAGATCAGAACCTACCAATGCAGCAGGAAACACCACACTGGGGGTTAC
CTCCGTTCTTCAGAGCACCTCAGTACCTTCTTCTGAGACCTGCCACGTCT
CCTACGAGGAGAGCAGAGTGGTCCTGGTGGTGGTGTACAGTGCCGTGTGC
CTGCTGGGCCTACCAGCCAACTGCCTGACTGCCTGGCTGACGCTGCTGCA
AGTCCTGCAGAGGAACGTGCTAGCCGTCTACCTGTTCTGCCTGTCCCTCT
GTGAGCTGCTCTACATCAGCACGGTGCCATTGTGGATCATCTACATCCAG
AATCAGCACAAATGGAACCTGGGTCCGCAGGCCTGCAAGGTGACTGCTTA
CATCTTCTTCTGCAACATCTACATCAGCATCCTCTTGCTCTGCTGCATTT
CCTGCGACCGCTACATGGCCGTGGTCTATGCACTGGAGAGCCGAGGCCAC
CGCCACCAGAGGACTGCTGTCACCATTTCTGCGTGTGTGATTCTTCTTGT
TGGACTTGTTAACTATCCAGTGTTTGACATGAAGGTGGAGAAGAGTTTCT
GCTTTGAGCCCCTGAGGATGAACAGCAAGATAGCCGGCTACCACTACCTG
CGTTTCACCTTTGGCTTTGCCATCCCTCTCGGCATCCTGGCGTTCACCAA
TCACCAGATCTTCCGGAGCATCAAACTCAGTGACAGCCTGAGCGCTGCGC
AGAAGAACAAGGTGAAGCGCTCCGCCATCGCGGTCGTCACCATCTTCCTG
GTCTGCTTTGCTCCCTACCACGTGGTACTCCTCGTCAAAGCTGCCAGCTT
TTCCTTCTACCAAGGAGACATGGATGCCGTGTGTGCCTTTGAAAGCAGAC
TGTACACAGTCTCTATGGTGTTTCTGTGCCTGTCTACAGTCAACAGTGTG
GCTGACCCCATCATCTACGTGCTGGGTACAGACCACTCTCGGCAAGAAGT
GTCCAGAATCCACACAGGGTGGAAAAAGTGGTCCACAAAGACATATGTTA
CATGCTCAAAGGACTCTGAGGAGACACACTTGCCCACAGAGCTTTCAAAC
ACATACACCTTCCCCAATCCCGCGCACCCTCCAGGATCACAGCCAGCGAA
GCTAGGTTTACTGTGCTCGCCAGAGAGACTGCCTGAGGAGCTCTGCTAA.

SEQ ID NO:16 is the nucleotide sequence of p4X-CRE-BLA-X.

### 7. DETAILED DESCRIPTION

The invention provides, in part, cells and methods for screening or characterizing signaling pathways and signaling pathway components. In particular embodiments, the invention provides, in part, cells and methods for screening or characterizing G-protein coupled receptors (GPCRs), ligands for GPCRs, and compounds that modulate signal transduction (e.g., agonists and antagonists). Such receptors are cell surface receptors that typically contain seven transmembrane regions and that transduce signals (e.g., sensory, hormonal, and neurotransmitter signals) from extracellular environments to intracellular environments.

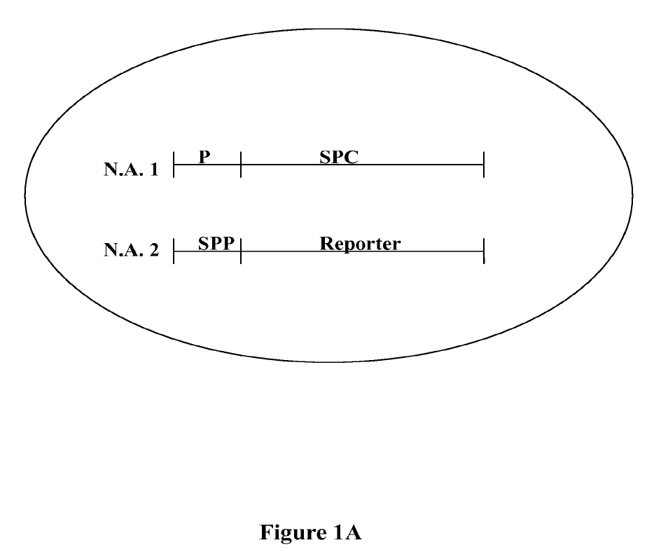

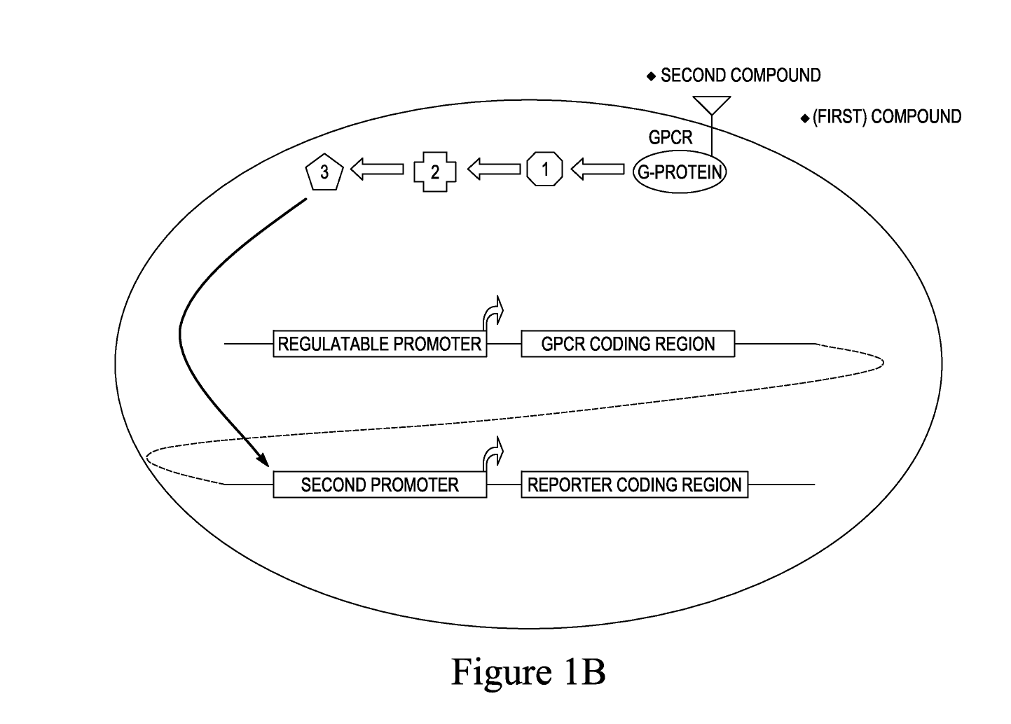

Using the illustrations in FIGS. 1A and 1B as examples, the invention includes, in part, method and compositions for detecting the inter-play between signaling pathways and a reporter. As shown in FIG. 1A a promoter (P) is operatively linked to a signaling pathway component (SPC). Thus, transcription based upon the promoter results in expression of the signaling pathway component. Further, a signaling pathway promoter (SPP) is operatively linked to a reporter coding sequence. The system in FIG. 1A is designed such that activation of the signaling pathway component results in a change in transcription levels of the reporter. One example of a signaling pathway component is a G2A GPCR.

The inventors have, inter alia, developed cell lines (e.g., stable cell lines) that are capable of expressing a GPCR (e.g., G2A). In one embodiment, the cells contain a nucleic acid comprising a GPCR coding region (e.g., for G2A) that is operatively linked and under the control of a regulatable promoter. This allows cells to be cultured with no or low levels of the GPCR being expressed and when desired the cell can be caused (e.g., induced) to express the GPCR. The constitutive expression of some cell signaling receptors is toxic to a cell and/or inhibits cell growth. In some cases the cell signaling receptor is toxic when expressed in an active state. Because of the low levels or absence of signaling receptor (e.g., a GPCR) expression from the cells of the invention, the cells can be cultured even if the expressed signaling receptor is toxic to the cell. Therefore, provided herein are methods of creating and/or producing cell lines (e.g., stable cell lines) expressing GPCRs. In some embodiments, the GPCRs are toxic and/or inhibit the development of a stable cell line when expressed, e.g., at higher levels that are desired for GPCR assays. Additionally, the present invention provides methods of using these cells in various GPCR assays that are known in the art or as provided herein.

The inventors have also made another surprising finding. When expressed (e.g., from a regulatable promoter), a GPCR (e.g., G2A) can be expressed as a constitutively activated GPCR even in the absence of its ligand. Therefore, the invention provides methods for expressing a constitutively active GPCR in a cell (e.g., a stable cell line) and methods for GPCR assays utilizing these cells. The invention provides particularly useful methods related to GPCRs that are toxic when expressed or overexpressed in a cell. The invention also includes methods for producing GPCRs which are constitutively active, as well as compositions (e.g., cells, etc.) which contain such GPCRs.

Cells which express a constitutively active GPCR can be readily utilized, for example, to screen or determine ligands that are agonists, inverse agonists, or inhibitors or enhancers of GPCR activation. This gives the advantage of being able to analyze the effects of the activated form of the GPCR without needing to provide or even knowing the ligand(s) for the GPCR. This invention also provides methods of identifying or evaluating compounds that modulate the activation state of a GPCR without needing to provide or even knowing the ligand(s) for the GPCR. If a ligand is available for the GPCR then the cell line can also be used to screen for antagonists. Modulators of GPCRs identified by methods described herein may directly interact with the GPCR or with one or more components and/or end products of the GPCR signaling pathway. In one embodiment, the cells are used for drug screening, e.g., screening for a drug that modulates a SPC and/or the signal pathway.

### Definitions

The terms “activated SPC” or “activated GPCR” refers to compounds, usually proteins, that are able to activate the next step (e.g., phosphorylate a protein) in a cellular pathway. For example, a GPCR may be considered activated or in an activated state when it is capable of activating a coupled G-protein and/or activating a cellular pathway. For clarity, when embodiments of the invention are discussed as having an activated SPC or GPCR, it is understood that not all of the expressed SPC or GPCR is necessarily expressed in an active state. In some embodiments, there is enough SPC or GPCR expressed in an active state to detect activation of the corresponding pathway(s).

“Compound” and “factor” are used interchangeably and are used in accordance with their art recognized meeting. A compound can be any chemical, nonlimiting examples include an inorganic chemical, an organic molecule, a protein or polypeptide, a carbohydrate, a polynucleotide, a polysaccharide, a lipid, a phospholipid, or a combination thereof. The term “test compound” refers to a compound to be tested by one or more screening methods of the invention, e.g., to determine if it is a putative modulator of a GPCR. Typically, various predetermined concentrations (e.g., various dilutions) of compounds are used for screening, such as 0.01 micromolar, 1 micromolar, or 10 micromolar. Experimental controls for a test compound can include measuring a signal for an assay performed in the absence of the test compound, with a different concentration of the compound (e.g., higher or lower), or comparing a signal obtained using a compound known to modulate a target activity with a signal obtained with the test compound.

A “construct,” when used in the context of molecular biology, is any genetically engineered nucleic acid (e.g., a plasmid, restriction fragment, a viral vector nucleic acid or an engineered chromosome).

A promoter is considered to be “modulated” by a GPCR (e.g., an active GPCR) when the expression of a coding region (e.g., for a reporter polypeptide) to which the promoter is operatively linked is either increased or decreased upon activation of the corresponding G-protein and/or a promiscuous G-α-protein. It is not necessary that the GPCR or even a G-protein activated by the GPCR directly modulate reporter gene expression. For example, other downstream events like changes in intracellular calcium levels or cAMP levels can more directly affect the expression of the reporter polypeptide.

“Promiscuous G-α-protein” refers to a protein with the promiscuous coupling activity of one of the G-α-proteins. In one embodiment, a promiscuous G-α-protein can couple to at least one GPCR that normally couples to a G-α-protein other than a promiscuous G-α-protein. In some embodiments, a promiscuous G protein is one that can couple to multiple GPCR types, e.g., Gs coupled receptors, Gq coupled receptors, and Gi/o coupled receptors. Examples of G-α-proteins, include G-α-q, G-α-s, G-α-i and G-α-12. Promiscuous G-α-protein coupling activity can be measured with an endogenously or heterologously expressed GPCR using the assays described herein. In some embodiments of the invention, a promiscuous G-α-protein can couple to at least two different types of GPCRs that normally couple to one of the following G-α-proteins, G-α-q, G-α-s, G-α-i and G-α-12. In other embodiments, a promiscuous G-α-protein can couple to at least three different types of GPCRs that normally couple to one of the following G-α-proteins, G-α-q, G-α-s, G-α-i and G-α-12. Promiscuous G-α-proteins permit coupling under conditions that would not occur with a G-α-protein and a receptor of a different G-α-subtype, unless the G-α-protein was expressed at sufficiently high levels to promote coupling with a GPCR that is not its normal coupling partner. Examples of G-α-15 are described in Wilke et al. (PNAS 88: 10049-10053, (1991)) and G-α-16 described in Amatruda et al. (PNAS 88: 5587-5591, (1991)). It is understood that promiscuous G-α-proteins do not include members of G-α-q, G-α-s, G-α-i and G-α-12 proteins that couple to only one type of GPCR.

The term “promoter” is used in accordance with its art recognized definition. A “promoter” is a sequence sufficient to direct transcription of a coding region or gene (including a cDNA encoding a protein) in an eukaryote. This includes a minimal sequence sufficient to direct transcription of a coding region or gene. In one embodiment, the promoter is derived from an eukaryotic gene or a virus that can direct transcription in an eukaryotic cell. A promoter can include, but is not limited to, a TATA box, a CAAT box, at least one response elements (e.g., a NFAT response element) and a transcriptional start site.

“Reporter coding regions” or “a coding region for a reporter polypeptide” refers to a nucleotide sequence encoding a polypeptide that is detectable either by its presence or activity, including, but not limited to, luciferase, a fluorescent protein (e.g., a green fluorescent protein), chloramphenicol acetyl transferase, beta-galactosidase, secreted placental alkaline phosphatase, beta-lactamase, human growth hormone, and other secreted enzyme reporters. Generally, reporter coding regions encode a polypeptide not otherwise produced by the host cell, which is detectable by analysis of the cell(s), e.g., by fluorometric, radioisotopic or spectrophotometric analysis of the cell(s). In one embodiment, the detection is performed without the need to kill the cells for signal analysis. In one embodiment, the coding region for a reporter polypeptide encodes an enzyme, which produces a change in fluorometric properties of the host cell. In some embodiments, the detected property is detectable by qualitative, quantitative or semi-quantitative (e.g., a function of transcriptional activation). Exemplary enzymes include esterases, phosphatases, proteases (e.g., tissue plasminogen activator or urokinase) and other enzymes whose function can be detected by appropriate chromogenic or fluorogenic substrates. In some embodiments, a reporter polypeptide utilizes a substrate to produce a detectable signal.

“Signaling pathway” refers to cellular signal transduction systems in which a stimulus external to a cell results in transcription inside the cell. Examples of signaling pathways include GPCR mediated pathways, hormone mediated pathways, kinase mediated pathways, a nuclear receptor mediated pathways, an ion channel mediated pathways or a G-protein mediated pathways. The signal of a signaling pathway may be transmitted across the cellular membrane, as occurs with GPCRs, or a signaling molecule may pass through the cell membrane, as with steroid hormone mediated signaling systems.

“Signaling pathway components” refers to members of signaling pathways. Typically, these members will function in the process of transmitting the signal. By way of example, signaling pathway components of a GPCR mediated signaling pathway may include the GPCR and the G-protein. In many instances, a signaling pathway component will be a protein.

“Signal transduction detection system” refers to a system for detecting signal transduction across a cell membrane, typically a cell plasma membrane. Such systems typically detect at least one activity or physical property directly or indirectly associated with signal transduction. For example, an activity or physical property directly associated with signal transduction can be the activity or physical property of either the receptor (e.g., GPCR), or a coupling protein (e.g., a G-protein). Signal transduction detection systems for monitoring an activity or physical property directly associated with signal transduction, include GTPase activity, and conformational changes. An activity or physical property indirectly associated with signal transduction is the activity or physical property produced directly by a molecule (other than by a receptor (e.g., GPCR)) and associated with a receptor (e.g., GPCR), or a coupling protein (e.g., a G-protein). Such indirect activities and properties include changes in intracellular levels of molecules (e.g., ions (e.g., Ca, Na or K)), second messenger levels (e.g., cAMP, cGMP and inositol phosphate), kinase activities, transcriptional activities, enzymatic activities, phospholipase activities, ion channel activities and phosphatase activities. Signal transduction detection systems for monitoring an activity or physical property indirectly associated with signal transduction, include transcriptional-based assays, enzymatic assays, intracellular ion assays and second messenger assays.

### GPCRs

GPCRs generally span cell membranes. Typically, a matching natural ligand binds to a GPCR's active site and causes a conformational change in the protein to form its active state and therefore activate the GPCR. This signals the G-protein coupled to the receptor inside the cell to release components that set some predefined cellular mechanism in motion.

Based upon current classification schemes, structurally, GPCRs can be divided into subfamilies, each of which currently includes orphan receptors as well as receptors whose ligands are characterized, e.g., reviewed in Gether (Endocrine Reviews 21:90-113 (2000)); and see Spedding et al. (International Union of Pharmacology. XXXI. Pharmacol. Rev, 54:231-232 (2002)). Fredricksson, et al. (Mol. Pharmacol., 63:1256-1272 (2003)) describe five main GPCR families, named glutamate, rhodopsin, adhesion, frizzled/taste2, and secretin, forming the GRAFS classification system. In one embodiment, the GPCR is from the glutamate, rhodopsin, adhesion, frizzled/taste2, or secretin family.

In one embodiment, the GPCR is a member or a derivative of a member of the Rhodopsin/β2 adrenergic receptor-like family of GPCRs including, but are not limited to, receptors for biogenic amines (e.g., adrenergic, serotonin, dopamine, muscarinic, histamine and the like), CCK, endothelin, tachykinin, neuropeptide Y, TRH, neurotensin, bombesin, growth hormone secretagogues, vertebrate and invertebrate opsins, bradykinin, adenosine, cannabinoid, melanocortin, olfactory signals, chemokines, FMLP, c5A, GnRH, eicosanoid, leukotriene, FSH, LH, TSH, fMLP, galanin, nucleotides, opioids, oxytocin, vasopressin, somatostatin and melatonin, as well as GPCRs activated by proteases.

In one embodiment, the GPCR is a member or a derivative of a member of the Glucagon/VIP/Calcitonin receptor-like family of GPCRs including, but are not limited to, receptors for calcitonin, CGRP, CRF, PTH, PTHrP, glucagon, glucagon-like peptide, GIP, GHRH, PACAP, VIP, secretin and latrotoxin.

In one embodiment, the GPCR is a member or a derivative of a member of the Metabotropic neurotransmitter/Calcium receptor family of GPCRs, which include, but are not limited to, metabotropic glutamate receptors, metabotropic GABA receptors, calcium receptors, vomeronasal pheromone receptors and taste receptors.

Databases containing links to the nucleotide sequences, amino acid sequences and other information related to numerous GPCRs, including orphan GPCRs, are available at http://www.gpcr.org/7tm/ (GPCRDB) at the CMBI, the Netherlands (formerly at the EMBL), http://tinygrap.uit.no/ (GRAP) Mutant Database at Tromso, Norway, The http://senselab.med.yale.edu/senselab/ORDB/ Olfactory Database (ORDB) at Yale, and Swiss-Prot (http://www.expasy.ch/).

The invention can be practiced with a nucleic acid encoding any GPCR, including variants and mutants of known GPCRs, or any desired fragment thereof. In many instances, a coding region for a GPCR(s) used in the practice of the invention will be introduced into cells. In some instances, a GPCR(s) used in the practice of the invention will be naturally resident in the cells (e.g., the wild-type gene located on one of the cell's chromosomes).

In particular embodiments of the invention, a GPCR, or other signaling pathway component, may be expressed as a fusion protein, e.g., with its peptide ligand, a transcription factor, with an arrestin, or with a G-protein α-subunit. Methods of recombinantly preparing functional GPCR-Gα fusions are known in the art (reviewed in Seifert et al., Trends Pharmacol. Sci. 20:383-389 (1999)). Constructs encoding other desired fusion proteins can be made by routine molecular biological methods. GPCRs, or other signaling pathway components, of the invention can be an epitope tagged version or a non-epitope tagged version.

One skilled in the art can readily obtain or isolate GPCR coding regions, as well as other signaling pathway component coding regions. For example, isolating a desired GPCR from a cDNA library (e.g., a commercial library). Additionally, GPCR coding regions can be obtained by reverse transcription PCR using primers specific for the GPCR. GPCR nucleotide and amino acid sequences are known in the art and readily available. With a known GPCR amino acid sequence one skilled in the art can also construct a synthetic (e.g., from overlapping oligos) GPCR coding region.

Various aspects of the present invention relate to cells expressing a GPCR (or other signaling pathway component), methods of expressing a GPCR (or other signaling pathway component) and GPCR (or other signaling pathway component) related assay methods. The present invention can be utilized with any GPCR or other signaling pathway component. In one embodiment, a GPCR employed is a class A (e.g., a “rhodopsin-like” receptor); a class B (e.g., a “secretin-like” receptor); a class C (e.g., a “metabotropic glutamate-like” receptor); a Frizzled and Smoothened-related receptor; an adhesion receptor family (e.g., a EGF-7™/LNB-7™ receptor); an adiponectin receptor or related receptor; or a chemosensory receptor including, but not limited to, an odorant, taste, vomeronasal or pheromone receptor. As examples, the GPCR superfamily in humans includes, but is not limited to, those receptor molecules described by Vassilatis, et al., Proc. Natl. Acad. Sci. USA, 100:4903-4908 (2003); Takeda, et al., FEBS Letters, 520:97-101 (2002); Fredricksson, et al., Mol. Pharmacol., 63:1256-1272 (2003); Glusman, et al., Genome Res., 11:685-702 (2001); and Zozulya, et al., Genome Biol., 2:0018.1-0018.12 (2001). In one embodiment, a GPCR(s) being assayed is a known GPCR(s). In one embodiment, a native and/or a non-native ligand(s) for the GPCR, or other signaling pathway component, is known. In one embodiment, a native and/or a non-native ligand(s) for the GPCR, or other signaling pathway component, is not known (e.g., an orphan GGPC). A GPCR used in the practice of the invention may be a GPCR of known function or of unknown function (e.g., an orphan GPCR).

In some aspects of the invention, the GPCR is a member of the secretin receptor family including, but not limited to, CALCR, NP_{—}001733.1, 7q21.3; CALCRL, NP_{—}005786.1, 2q21.1-q21.3; CRHR1, NP_{—}004373.1, 17q21.31; CRHR2, NP_{—}001874.1, 7p14.3; GCGR, NP_{—}000151.1, 17q25.3; GHRHR, NP_{—}000814.1, 7p14; GIPR, NP_{—}000155.1, 19q13.3; GLP1R, NP_{—}002053.1, 6p21.2; GLP2R, NP_{—}004237.1, 17p1.2; PACAP, NP_{—}001109.1, 7p14; PTHR1, NP_{—}000307.1, 3p21.31; PTHR2, NP_{—}005039.1, 2q33; SCTR, NP_{—}002971.1, 2q14.1; VIPR1, NP_{—}004615.1, 3p22.1; and VIPR2, NP_{—}003373.1, 7q36.3.

In some aspects of the invention, the GPCR is a member of the adhesion receptor family including, but not limited to, BAI1, NP_{—}001693.1, 8q24; BAI2, NP_{—}001694.1, 1p35; BAI3, NP_{—}001695.1, 6q12; CELSR1, NP_{—}055061.1, 22q13.3; CELSR2, NP_{—}001399.1, 1p21; CELSR3, NP_{—}001398.1, 3p21.31; CD97, NP_{—}001775.1, 19p13.13; EMR1, NP_{—}001965.1, 19p13.3; EMR2, NP_{—}038475.1, 19p13.1; EMR3, NP_{—}115960.1, 19p13.3; ETL, NP_{—}071442.1, 1p33-p32; GPR97, AY140959, 16q13; GPR110, AY140952, 6p12.3; GPR111, AY140953, 6p12.3; GPR112, AY140954, Xq26.3; GPR113, AY140955, 2p23.3; GPR114, AY140956, 16q13; GPR115, AY140957, 6p12.3; GPR116, AY140958, 6p12.3; HE6 (GPR64), NP_{—}005747.1, XP22.22; LEC1, NP_{—}036434.1, 1p31.1; LEC2, NP_{—}055736.1, 19p13.2; LEC3, NP_{—}056051.1, 4q13.1; and GPR56 (TMVIIXN1), NP_{—}003263.1, 1q42-q43.

In some aspects of the invention, the GPCR is a member of the glutamate receptor family including, but not limited to, CASR, NP_{—}000379.1, 3q21.1; GABBR1, NP_{—}001461.1, 6p21.1; GABBR2(GPR51), NP_{—}005449.1, 9q22.1-q22.3; GRM1, NP_{—}000829.1, 6q24.3; GRM2, NP_{—}000830.1, 3p21.31; GRM3, NP_{—}000831.1, 7q21.12; GRM4, NP_{—}000832.1, 6p21.1; GRM5, NP_{—}000833.1, 11q21.1; GRM6, NP_{—}000834.1, 5q35.3; GRM7, NP_{—}000835.1, 3p21.1; GRM8, NP_{—}000836.1, 7q31.3-q32.1; GPRC6A, NP_{—}683766.1, 6q22.1; TAS1R1, NP_{—}619642, 1p36.23; TAS1R2, NP_{—}689418.1, 1p36.2; and TAS1R3, XP_{—}060177.1, 1p36.33.

In some aspects of the invention, the GPCR is a member of the frizzled/taste2 receptor family including, but not limited to, FZD1, NP_{—}003496.1, 7q21.13; FZD2, NP_{—}001454.1, 17q21.31; FZD3, NP_{—}059108.1, 8p21.1; FZD4, NP_{—}036325.1, 11q14.2; FZD5, NP_{—}003459.1, 2q33-q34; FZD6, NP_{—}003497.1, 8q22.3-q23.1; FZD7, NP_{—}003498.1, 2q33; FZD8, NP_{—}114072.1, 10p1.21; FZD9, NP_{—}003459.1, 7q11.23; FZD10, NP_{—}009128.1, 12q24.33; SMOH, NP_{—}005622.1, 7q32.1; TAS2R13, NP_{—}076409, 12p13; TAS2R14, NP_{—}076411.1, 12p13; TAS2R7, NP_{—}076408.1, 12p13; TAS2R9, NP_{—}076406.1, 12p13; TAS2R8, NP_{—}76407.1, 12p13.2; TAS2R3, NP_{—}058639.1, 7q31.3-q32; TAS2R10, NP_{—}076410.1, 12p13; TAS2R5, NP_{—}061853.1, 7q31.3-q32; TAS2R4, NP_{—}058640.1, 7q31.3-q32; TAS2R1, NP_{—}062545.1, 5p15; TAS2R16, NP_{—}58641.1, 7q31.1-q31.3; GPR59, XP_{—}069626, 7q33; and GPR60, XP_{—}090424, 7q33.

In some aspects of the invention, the GPCR is a member of the rhodopsin receptor family including, but not limited to, TBXA2R, NP_{—}001051.1, 19p13.3; PTGER3, NP_{—}000948.1, 1p31; PTGER2, NP_{—}000947.1, 1 q22.1; PTGDR, XP_{—}051711.1, 14q22.1; PTGER4, NP_{—}000949.1, 5p12; PTGIR, NP_{—}000951.1, 19q13.31; PTGER1, NP_{—}000946.1, 19p13.12; PTGFR, NP_{—}000950.1, 1p31.1; SREB3, NP_{—}061842.1, Xp11; GPR26, XP_{—}061555.1, 10q26.2; SREB1(GPR27), NP_{—}061844.1, 3p21-p14; SREB2(GPR85), NP_{—}061843.1, 7q31; GPR61, NP_{—}114142, 1p13.3; GPR62, NT_{—}005975.6, 3p21.31; GPR78, NT_{—}006307.5, 4p16.1; HTR1A, NP_{—}000515.1, 5q11.2-q13; HTR5(HTR5A), NP_{—}076917.1, 7q36.3; HTR7, NP_{—}000863.1, 10q21-q24; HRH2, NP_{—}071640.1, 5q35.2; HTR4, NP_{—}000861.1, 5q31-q33; HTR6, NP_{—}000862.1, 1p36-q35; ADRA1A, NP_{—}000671.1, 8p21.2; ADRA1D, NP_{—}000669.1, 20p13; ADRA1B, NP_{—}000670.1, 5q33.1; ADRB1, NP_{—}000675.1, 10q25.3; ADRB3, NP_{—}000016.1, 8p12-p11.2; ADRB2, NP_{—}000015.1, 5q32; DRD5, NP_{—}000789.1, 4p16.1; DRD1, NP_{—}000785.1, 5q35.2; HTR2B, NP_{—}000858.1, 2q36.3-q37.1; HTR2A, NP_{—}000612.1, 13q14-q21; HTR2C, NP_{—}000859.1, Xq24; TAR1, AAK71236; 8q23.2; PNR, NP_{—}003958.1, 6q23; TAR3, AAK71240; 6q23.2; TAR4, AAK71243; 6q23.2; TAR5(GPR102), NP_{—}444508.1, 6q23.2; GPR58, NP_{—}055441.1, 6q24; GPR57, NP_{—}055442.1, 6q23.2; HTR1B, NP_{—}000854.1, 6q13; HTR1D, NP_{—}008555.1, 1p36.3-p34.3; HTR1E, NP_{—}000856.1, 6q14-q15; HTR1F, NP_{—}000857.1, 3p12; ADRA2B, NP_{—}000673.1, 3p13-q13; ADRA2A, NP_{—}000672.1, 10q25.2; ADRA2C, NP_{—}000674.1, 4p16; DRD4, NP_{—}000788.1, 11p15.5; DRD3, NP_{—}000787.1, 3q13.3; DRD2, NP_{—}000786.1, 11q23, HRH4, NP_{—}067830.1, 18q11.2; CHRM4, NP_{—}000732.1, 11p12-p11.2; CHRM2, NP_{—}000730.1, 7q31-q35; CHRM1, NP_{—}000729.1, 11q13; CHRM3, NP_{—}000731.11q43; CHRM5, NP_{—}036257.1, 15q26; ADORA3, NP_{—}000668.1, 1p13.3; ADORA1, NP_{—}000671.1, 8p21.2, ADORA2A, NP_{—}000666.1, 22q11.23; ADORA2B, NP_{—}000667.1; 17q12; GPR3, NP_{—}005272.1, 1p35.3; GPR12, NP_{—}005279.1, 13q12.13; GPR6, NP_{—}005275.1, 6q21; MC2R, NP_{—}000520.1, 18p11.2; MC1R, NP_{—}002377.1, 16q24.3; MC3R, NP_{—}063941.1, 20q13.31; MC4R, NP_{—}005903.1, 18q22; MC5R, NP_{—}005904.1, 18p11.2; EDG7, NP_{—}036284.1, 1p22.3; EDG2, NP_{—}001392.1, 9q31.3; EDG4, NP_{—}004711.1, 19p12; EDG8, NP_{—}110387.1, 19p13.2; EDG5, NP_{—}004221.1, 19p13.2; EDG6, NP_{—}003766.1, 19p13.3; EDG3, NP_{—}005217.1, 9q22.1; EDG1, NP_{—}001391.1, 1p21; CNR1, NP_{—}001831.1, 6q15; CNR2, NP_{—}001832.1, 1p36.11; AVPR2, NP_{—}000045.1, Xq28; AVPR1A, NP_{—}000697.1, 12q14.1; AVPR1B, NP_{—}000698.1, 1q32; EDNRB, NP_{—}000106.1, 13q22.3; EDNRA, NP_{—}001948.1, 4q31.21; ETBRLP1 (GPR37), NP_{—}005293.1, 7q31; ETBRLP2, NP_{—}004758, 1q31.3; BRS3, NP_{—}001718.1, Xq21-q28; CCKAR, NP_{—}000721.1, 4p15.1-p15.2; CCKBR, NP_{—}000722.1, 11p15.4; Ghrelin(GPR38), NP_{—}001498.1, 13q14-q21; GHSR, NP_{—}004113.2, 3q26.2; GNRHR, NP_{—}000397.1, 4q21.2; GNRHRII, NP_{—}476504.1, 1q12; GRPR, NP_{—}005302.1, Xp22.1-p22.13; HCRTR2, NP_{—}001517.1, 6p12.1; HCRTR1, NP_{—}001516.1, 1p33; NTSR1, NP_{—}002522.1, 20q13; NTSR2, NP_{—}036476.1; NMU2R, NP_{—}064552.1, 5q33.2; NMU1R(GPR66), NP_{—}006047.1, 2q37.1; NMBR, NP_{—}002502.1, 6q24.1; OXTR, NP_{—}000907.1, 3p25; NPFF1, NP_{—}071429.1, 1q21-q22; NPFF2(GPR74), NP_{—}004876.1, 4q21; TACR2, NP_{—}001048.1, 10q22.1; TACR3, NP_{—}001050.1, 4q25; TACR1, NP_{—}001049.1, 2p13.1; TAC3RL, NP_{—}006670.1; NPY5R, NP_{—}006165.1, 4q31-q32; PPYR1, NP_{—}005963.1, 10q11.21; NPY1R, NP_{—}000900.1, 4q31.3; PrRP (GPR10), NP_{—}004239.1, 10q25.3-q26; GPR72, NP_{—}057624.1, 11q21; NPY2R, NP_{—}000901.1, 4q31; GPR54, NP_{—}115940.1, 19p13.3; GALR1, NP_{—}001471.1, 18q23; GALR2, NP_{—}003848.1, 17q25.3; GALR3, NP_{—}003605.1, 22q13.1; GPR8, NP_{—}000836.1, 7q31.3-q32.1; GPR7, NP_{—}000835.1, 3p26.1; OPRL₁, NP_{—}000904.1, 20p13.3; OPRD1, NP_{—}000902.1, 1p36.1-p34.3; OPRM1, NP_{—}000905.1, 6q25.2; OPRK1, NP_{—}000903.1, 8q11.23; SSTR3, NP_{—}001042.1, 22q13.1; SSTR5, NP_{—}001044.1, 16p13.3; SSTR2, NP_{—}001041.1, 17q25.1; SSTR1, NP_{—}061842.1, Xp11; SSTR4, NP_{—}001043.1, 20p11.2; RDC1, NP_{—}051522.1, 2q37.3; AGTRL1, NP_{—}005152.1, 11q12.1; GPR1, NP_{—}005270.1, 2q33.3; CRTH2(GPR44), NP_{—}004769.1, 11q12.2; AGTR2, NP_{—}000677.1, Xq23; ADMR, NP_{—}009195.1, 12q32.3; AGTR1, NP_{—}000646.1, 3q24; CCR7, NP_{—}001829.1, 17q21.2; CCR6, NP_{—}004358.1, 6q27; CXCR6, NP_{—}006555.1, 3p21; CCR9, NP_{—}006632.2, 3p21.31; CCR11, NP_{—}057641.1, 3p21.31; CXCR4, NP_{—}003458.1, 2q21.3; CCR8, NP_{—}005192.1, 3p22.2; CCRL2, NP_{—}003956.1, 3p21.31; CXC3R1, NP_{—}001328.1, 3p22.2; CCR4, NP_{—}005499.1, 3p24; CCR1, NP_{—}001286.1, 3p21.31; CCR3, NP_{—}001828.1, 3p21.31; CCR2, NP_{—}000639.1, 3p21.31; CCR5, NP_{—}000570.1, 3p21.31; XCR1(CCXCR1), NP_{—}005274.1, 3p21.3; CCBP2, NP_{—}001287.1, 3p21.31; CXCR5, NP_{—}001707.1, 11q23.3; CCR10(GPR2), NP_{—}057687.1, 17q21.31; CXCR3(GPR9), NP_{—}001495.1, Xq13; CXCR1(IL8RA), NP_{—}000625.1, 2q35; CXCR2(IL8RB), NP_{—}001548.1, 2q35; BDKRB1, NP_{—}000701.1, 14q32.2; BDKRB2, NP_{—}000614.1, 14q32.2; CMKLR1, NP_{—}004063.1, 12q23.3; C5L2(GPR77), NP_{—}060955.1, 19q13.3; C5R1, NP_{—}001727.1, 19q13.32; GPR32, NP_{—}001497.1, 19q13.3; FPR1, NP_{—}002020.1, 19q14.4; FPRL2, NP_{—}002021.1, 19q13.3; FPRL1, NP_{—}001453.1, 19q13.3; GPR25, NP_{—}005289.1, 1q32.1; GPR15, NP_{—}005281.1, 3q12.1; BLTR2, NP_{—}062813.1, 14q11.2; BLTR(LTB4R), NP_{—}000743.1, 14q11.2; SALPR, NP_{—}057652.1, 5p15.1-p14; MAS, NP_{—}002368.1, 6q25.3; MRGF, AAH16964, 11q12.1; MRGX2, NP_{—}473371.1, 11p15.1; MRGX1, NP_{—}089843.1, 11p15.1; MRGX4, NP_{—}473373.1, 11p15.1; MRGX3, NP_{—}473372.1, 11p15.1; MRGD, XP_{—}089955.1, 11q12.2; MRG, NP_{—}443199.1, 6p21.1; LGR8, NP_{—}570718.1, 13q13.2; LGR7, NP_{—}067647.1, 4q32; LGR4(GPR48), NP_{—}060960.1, 11p14.1; LGR6, XP_{—}046692.1, 1q32.1; LGR5(GPR49), NP_{—}003658.1, 12q22-q23; LHCGR, NP_{—}000224.1, 2p16.3; FSHR, NP_{—}000136.1, 2p16.3; TSHR, NP_{—}000360.1, 14q31.1; GPR18, NP_{—}005283.1, 13q32; PTAFR, NP_{—}000943.1, 1p36.11; G2A, NP_{—}037477.1, 14q32.3; EBI2, NP_{—}004942.1, 13q32.3; P2Y11(P2RY11), NP_{—}002557.1, 19p13.2; GPR92, NP_{—}065133.1, 12p13.31; C3AR(C3AR1), NP_{—}004045.1, 12p13.31; P2Y9(GPR23), NP_{—}005287.1, Xq21.31; P2Y5, NP_{—}005758.1, 13q14.2; FKSG79, NP_{—}115942.1, Xq21.1; P2Y10, NP_{—}055314.1, Xq21.1; GPR17, NP_{—}005282.1, 2q14.3; F2RL3, NP_{—}003941.1, 19p13.11; F2RL2, NP_{—}004092.1, 5q13.1; F2R, NP_{—}001983.1, 5q13.1; F2RL1, NP_{—}005233.1, 5q13.1; GPR87, NP_{—}076404.1, 3q25.1; GPR105, NP_{—}055694.1, 3q25.1; P2Y12, NP_{—}073625.1, 3q25.1; FKSG77(GPR86, GPR94), NP_{—}076403.1, 3q25.1; CYSLT1, NP_{—}006630.1, Xq21.1; CYSLT2, NP_{—}065110.1, 13q14.2; GPR80(GPR99), XP_{—}062888.1, 13q32.1; GPR91, NP_{—}149039.1, 3q25.1; P2Y6(P2RY6), NP_{—}004145.1, 11q14.1; P2Y1(P2RY1), NP_{—}002554.1, 3q25.2; P2Y2(P2RY2), NP_{—}002555.1, 11q13.1; P2Y4(P2RY4), NP_{—}002556.1, Xq13.1; FKSG80(GPR81), NP_{—}115943.1, 12q24.31; HM74, NP_{—}006009.1, 12q24.31; GPR35, NP_{—}005292.1, 2q37.3; GPR55, NP_{—}005674.1, 2q37; GPR65, NP_{—}003599.1, 14q31.3; OGR1(GPR68), NP_{—}003476.1, 14q31; GPR4, NP_{—}005273.1, 19q13.3; H963, NP_{—}037440.1, 3q25.1; GPR82, NP_{—}543007.1, 1; TRHR, NP_{—}003292.1, 8p23; RE2, NP_{—}031395.1, 1p36.13-q31.3; GPR103, NT_{—}006337.5, 4q26; RGR, NP_{—}002912.1, 10q22.3; GPR101, NP_{—}473362.1, Xq26.3; GPRC5B, NP_{—}071319, 17q25; GPRC5C, NP_{—}016235.1, 16p12; GPRC5D, NP_{—}061124.1; GPR, NP_{—}009154.1, 15q13.3; GPR14, NP_{—}061822.1, 17q25.3; GPR19, NP_{—}006134.1, 12p12.3; GPR20, NP_{—}005284.1, 8q24.2-q24.3; GPR22, NP_{—}005286.1, 7q22-q31.1; CMKRL2(GPR30), NP_{—}001496.1, 7p22; GPR31: NP_{—}005290.1, 6q27; GPR34, NP_{—}005291.1, Xp11.4-p11.3; GPR40, NP_{—}005294.1, 19q13.12; GPR41(GPR42), NP_{—}005295.1, 19q13.12; GPR43, NP_{—}005297.1, 19q13.12; GPR39, NP_{—}001499.1, 2q21-q22; GPR63, NP_{—}110411.1, 6q16.1-q16.3; GPR75, NP_{—}006785.1, 2p16; GPR84, NP_{—}065103.1, 12q13.13; HRH1, NP_{—}000852.1, 3p25; HRH3, NP_{—}009163.1, 20q13.33; SREB2(GPR85), NP_{—}061843.1, 7q31; VLGR1, XP_{—}057299, 5q13; and V1RL1, NP_{—}065684, 19q13.43.

GPCRs that can be utilized with the invention are not limited to known GPCRs or wild-type GPCRs. By “wild-type GPCRs” is meant GPCRs with amino acid sequences corresponding to those found in nature. Mutant GPCRs can also be utilized in the present invention. Methods for creating and even screening mutant GPCRs for certain functions are well known in the art. For example, it is well within the skill of one skilled in the art to make mutations in a GPCR coding region and screen for GPCRs with desired characteristics. Mutation can be directed or random mutations. For example, if the sequence and/or structure of the GPCR is known, certain domains (e.g., ligand binding domain, transmembrane domain or a domain involved in G-protein activation) can be mutated. In some embodiments, when the cell signaling component is a GPCR, the GPCR could be mutated to have an increased ability for activating a G-protein. In this case the GPCR could be, for example, mutated to bind an agonist ligand with higher or lower affinity and/or enhanced binding or coupling with a G-protein. The present invention includes a mutant GPCR with one or more of these characteristics.

In one embodiment, the GPCR, or other cell signaling component, is not normally expressed in the parental cell. In one embodiment, the level of the GPCR, or other cell signaling component, expression in the cell is altered. For example, the parental cell may already express a GPCR and the present invention includes increasing or regulating expression of the GPCR. This may be accomplished, for example, by introducing a non-native (e.g., a regulatable promoter) upstream of the GPCR coding region. In another embodiment, at least one expression vector is introduced into the cell, wherein the expression vector is capable of increasing expression levels of the GPCR. In one embodiment, the GPCR coding region, or coding region of another cell signaling component, is operatively linked to a regulatable promoter in the expression vector. In other words, the invention includes any method of changing, increasing or decreasing expression of the GPCR or other cell signaling component. For example, inserting a non-native promoter upstream of a native GPCR.

Many embodiments of the invention will include a polynucleotide sequence not naturally occurring in the cell encoding a GPCR and a promiscuous G-protein (e.g., a promiscuous G-protein-α) construct. In one embodiment, the GPCR is not under the control of a promoter controlling a G-protein (e.g., a promiscuous G-α-protein). Promoters known in the art can be used to either constitutively or inducibly express the receptor or putative receptor.

GPCRs that can be used with the invention include, but are not limited to, muscarinic receptors, e.g., human M2 (GenBank accession#M16404); rat M3 (GenBank accession#M16407); human M4 (GenBank accession#M16405); human M5 (Bonner, et al., (1988) Neuron 1, pp. 403-410); and the like; neuronal nicotinic acetylcholine receptors, e.g., the human .alpha..sub.2, .alpha..sub.3, and .beta..sub.2, subtypes disclosed in U.S. Ser. No. 504,455 (filed Apr. 3, 1990); the human .alpha..sub.5 subtype (Chini, et al. (1992) Proc. Natl. Acad. Sci. U.S.A. 89: 1572-1576), the rat .alpha..sub.2 subunit (Wada, et al. (1988) Science 240, pp. 330-334); the rat .alpha..sub.3 subunit (Boulter, et al. (1986) Nature 319, pp. 368-374); the rat .alpha..sub.4 subunit (Goldman, et al. (1987) Cell 48, pp. 965-973); the rat .alpha..sub.5 subunit (Boulter, et al. (1990) I. Biol. Chem. 265, pp. 4472-4482); the chicken .alpha..sub.7 subunit (Couturier et al. (1990) Neuron 5: 847-856); the rat .beta..sub.2 subunit (Deneris, et al. (1988) Neuron 1, pp. 45-54) the rat .beta..sub.3 subunit (Deneris, et al. (1989) J. Biol. Chem. 264, pp. 6268-6272); the rat .beta..sub.4 subunit (Duvoisin, et al. (1989) Neuron 3, pp. 487-496); combinations of the rat .alpha. subunits, and s .beta. subunits and a and p subunits; GABA receptors, e.g., the bovine x, and .beta..sub.1, subunits (Schofield, et al. (1987) Nature 328, pp. 221-227); the bovine X.sub.2, and X.sub.3, subunits (Levitan, et al. (1988) Nature 335, pp. 76-79); the .gamma.-subunit (Pritchett, et al. (1989) Nature 338, pp. 582-585); the .beta..sub.2, and .beta..sub.3, subunits (Ymer, et al. (1989) EMBO J. 8, pp. 1665-1670); the 8 subunit (Shivers, B. D. (1989) Neuron 3, pp. 327-337); and the like; glutamate receptors, e.g., rat GluR1 receptor (Hollman, et al. (1989) Nature 342, pp. 643-648); rat GluR2 and GluR3 receptors (Boulter et al. (1990) Science 249:1033-1037; rat GluR4 receptor (Keinanen et al. (1990) Science 249: 556-560); rat GluR5 receptor (Bettler et al. (1990) Neuron 5: 583-595); rat GluR6 receptor (Egebjerg et al. (1991) Nature 351: 745-748); rat GluR7 receptor (Bettler et al. (1992) neuron 8:257-265); rat NMDAR1 receptor (Moriyoshi et al. (1991) Nature 354:31-37 and Sugihara et al. (1992) Biochem. Biophys. Res. Comm. 185:826-832); mouse NMDA el receptor (Meguro et al. (1992) Nature 357: 70-74); rat NMDAR2A, NMDAR2B and NMDAR2C receptors (Monyer et al. (1992) Science 256: 1217-1221); rat metabotropic mGluR1 receptor (Houamed et al. (1991) Science 252: 1318-1321); rat metabotropic mGluR2, mGluR3 and mGluR4 receptors (Tanabe et al. (1992) Neuron 8:169-179); rat metabotropic mGluR5 receptor (Abe et al. (1992) I. Biol. Chem. 267: 13361-13368); and the like; adrenergic receptors, e.g., human .beta.1 (Frielle, et al. (1987) Proc. Natl. Acad. Sci. 84, pp. 7920-7924); human .alpha..sub.2 (Kobilka, et al. (1987) Science 238, pp. 650-656); hamster .beta..sub.2 (Dixon, et al. (1986) Nature 321, pp. 75-79); and the like; dopamine receptors, e.g., human D2 (Stormann, et al. (1990) Molec. Pharm. 37, pp. 1-6); mammalian dopamine D2 receptor (U.S. Pat. No. 5,128,254); rat (Bunzow, et al. (1988) Nature 336, pp. 783-787); and the like; and the like; serotonin receptors, e.g., human 5HT1a (Kobilka, et al. (1987) Nature 329, pp. 75-79); serotonin 5HT1C receptor (U.S. Pat. No. 4,985,352); human 5HT1D (U.S. Pat. No. 5,155,218); rat 5HT2 (Julius, et al. (1990) PNAS 87, pp. 928-932); rat 5HT1c (Julius, et al. (1988) Science 241, pp. 558-564), GPCR-K2 (Benovic et al. J Biol Chem 262:9026-9032, 1987), formyl peptide receptor like-1 (FPRL-1) receptor (Murphy et al. 1992. J. Biol. Chem. 267:7637-7643; Ye, et al. 1992 Biochem. Biophys. Res. Commun. 184:582-589), a G2a GPCR, a hmGlu1a receptor (Lavreysen et al. 2002 Molecular Pharmacology 61(5) 1244-1254), a GPR23 receptor (O'Dowd et al. (1997) Gene 187, 75-81), a Histamine H4 (Nguyen et al., Mol Pharmacol. 2001 March; 59(3):427-33), a FPRL-2 receptor (Bao, et al. 1992. 19. Genomics 13:437-440), and the like. Also, see Steven et al., International Union of Pharmacology. XLVI. G Protein-Coupled Receptor List. Pharmacol Rev. 2005 June; 57(2):279-880) for other GPCRs that can be used with the invention.

The present invention also includes methods of screening wild-type GPCRs and/or mutant GPCRs as described herein.

### G2A GPCRs

In one embodiment, when a signaling pathway component is a GPCR, the GPCR is a G2A GPCR. In one embodiment, the GPCR is a mammalian or human G2A. In some embodiments, the G2A GPCR is from a species other than human. In some embodiments, a G2A GPCR is a murine G2a GPCR. One skilled in the art, utilizing common methods (e.g., BLAST and/or alignments and/or nucleic acid hybridization studies), can identify G2A GPCRs from other species.

G2A is believed to be an immunoregulatory GPCR predominately expressed in lymphocytes, monocytes, and macrophages and was named for its ability to function at the G-2/M checkpoint to delay mitosis resulting in accumulation of cells in the G2 phase (Rikitake, et al 2002 Arterioscler Thromb Vasc Biol. 22, 2049-2053; Weng, et al. 1998 Proc. Natl. Acad. Sci. USA 95, 12334-12339). G2A was originally thought to be a potential tumor suppressor, since over-expression of G2A was shown to suppress the ability of the oncogenic tyrosine kinase, Bcr-Abl, to transform pre-B cells and fibroblasts (Weng, et al. 1998). More recently, G2A has also been considered to be an oncogenic GPCR as over-expression caused transformation of NIH3T3 fibroblasts (Zohn, et al. 2000 Oncogene 19, 3866-3877). In addition, knockout studies have demonstrated that mice lacking the G2A receptor develop a late on-set autoimmune disease similar to the human autoimmune disease, systemic lupus erythematosus (SLE) (Le et al. (2001) Immunity 14, 561-571). G2A is thought to be a high-affinity receptor for lysophosphatidylcholine (LPC). LPC, a phospholipid component of oxidized LDL, plays an etiological role in atherosclerosis and has also been implicated in the pathogenesis of SLE (Lusis, A. J. (2000) Nature 407, 233-241; Wu, et al. (1999) Lupus 8, 142-150; Koh, et al (2000) J. Immunol. 165, 4190-4201). The therapeutic effects of LPC have been examined in mouse models of sepsis. One such study found that administration of LPC protected mice from lethality associated with cecal ligation and puncture (CLP) (Yan et al. (2004) Nature Medicine 10, 161-167). However, pre-treatment of the mice with an antibody to G2A inhibited this LPC-induced protection from CLP lethality. These results suggest that LPC could be used to prevent and/or treat sepsis and microbial infections in a G2A dependent manner (Yan et al. 2004).

GPCRs lead to activation of various signal transduction pathways through coupling to specific G proteins. Over-expression of G2A in HeLa cells has been described as stimulating the accumulation of inositol phosphates and cAMP (Lin, P., and Ye, R. D. (2003) J. Biol. Chem. 278, 14379-14386). LPC augmented the cAMP signal in a dose dependent manner, but did not further enhance the inositol phosphate accumulation. LPC has also been shown to lead to an increase in intracellular calcium concentrations in MCF10A epithelial cells overexpressing G2A.

Co-expression of Gαq and Gα13 with G2A in HeLa cells led to G2A-mediated activation of an NF-kB response element. Co-expression of LscRGS, a GTPase-activating protein that suppresses signaling by Gα13, inhibited G2A induced morphological changes in fibroblasts further supporting the coupling of G2A to Gα13 (Zohn, et al. 2000). In addition, G2A has been reportedly linked to the Gαi signaling pathway in MCF10A and CHO cells (Kabarowski, et al. (2001) Science 293, 702-705). These results show that G2A can couple to multiple G proteins including Gαs, Gαq, and Gα13, and Gαi (Kabarowski, et al. (2001); Lin, P., and Ye, R. D. (2003) J. Biol. Chem. 278, 14379-14386). It has also been reported that G2A is a proton-sensing GPCR and that lowering the pH augmented the inositol phosphate accumulation in G2A expressing cells (Murakami et al. (2004) J. Biol. Chem. 279, 42484-42497). Interestingly, it has been suggested that LPC dose-dependently inhibited the pH-dependent activation of G2A thus suggesting that LPC acts as an antagonist in this instance. In addition to the various signaling pathways activated by G2A, G2A has been shown to lead to apoptosis induction in HeLa, NIH3T3, COS-7, Saos2, and U20S and to induce chemotaxis in Jurkat cells.

The inventors have, inter alia, surprisingly developed cell lines (e.g., stable cell lines) that are capable of expressing a constitutively activated GPCR (e.g., G2A). In one embodiment, the cells contain a nucleic acid comprising a GPCR coding region (e.g., for G2A) that is operatively linked and under the control of a regulatable promoter. In one embodiment, the cells express a constitutively activated GPCR in the absence of the GPCR's ligand(s).

### GPR23 GPCRs

GPR23, also referred to as P2Y9 and LPA4, is a GPCR identified as a receptor for lysophosphatidic acid (LPA). GPR23 is structurally distinct from the other GPCRs that have been identified as receptors for LPA, e.g., LPA1, LPA2, and LPA3 (EDG-2, EDG-4 and EDG-7, respectively). GPR23 is believed to be coupled to Gq and Gs pathways, in contrast to the other LPA receptors (which are believed to be coupled to Gi and Go pathways). GPR23 sequence identity between species is high, with the human receptor having more than 96% identity with the murine receptor.

The scientific literature has reported that GPR23 is predominantly expressed in the ovaries, but expression in other organs and tissues has also been observed (see, e.g., Anliker et al., Biol. Chem. 279(20):20555-558 (2004) and Noguchi et al., J. Biol. Chem. 278(28):25600-606 (2003)). GPR23 has been connected to a number of diseases and disorders, including infections (e.g., viral infections), cancer, inflammatory disorders, cardiovascular disorders (e.g., heart failure and hypertension), urological disorders (e.g., urinary retention), and neurological disorders (e.g., anxiety and schizophrenia) (see, e.g., U.S. Pat. No. 6,010,877; PCT Publication No. WO 04/106936 and U.S. Patent Publication No. 20030064438) and metabolic disorders.

Inhibition or modulation of GPR23 signaling with a modulator (e.g., a small molecule antagonist, agonist, inverse agonist or an antibody) may be therapeutically beneficial in the treatment of any of the diseases listed above. GPR23 is further described in U.S. Patent Application No. 20060275285.

In some embodiments, a cell of the invention is engineered to express a GPR23 GPCR. In some embodiments, a GPR23 coding region is operatively linked to a regulatable promoter. In some embodiments, a cell expressing a GPR23 does not express another GPCR that is a LPA receptor. In some embodiments, a cell expressing a GPR23 expresses between from about 2 to about 100 fold, about 2 to about 3 fold, about 2 to about 4 fold, about 3 to about 5 fold, about 4 to about 6 fold, about 5 to about 7 fold, about 6 to about 8 fold, about 7 to about 9 fold, about 8 to about 10 fold, about 2 to about 10 fold, about 2 to about 50 fold, about 5 to about 10 fold, about 5 to about 20 fold, about 10 to about 25 fold, about 25 to about 50 fold, or about 50 to about 100 fold more GPR23 molecules that the other GPCR LPA receptors combined. In some embodiments, a cell expressing a GPR23 shows a response to a LPA or ligand of between from about 1.5 to about 3 fold, about 1.25 to about 2 fold, about 2 to about 100 fold, about 2 to about 3 fold, about 2 to about 4 fold, about 3 to about 5 fold, about 4 to about 6 fold, about 5 to about 7 fold, about 6 to about 8 fold, about 7 to about 9 fold, about 8 to about 10 fold, about 2 to about 10 fold, about 2 to about 50 fold, about 5 to about 10 fold, about 5 to about 20 fold, about 10 to about 25 fold, about 25 to about 50 fold, or about 50 to about 100 fold more than the response for the same LPA or ligand of the other GPCR LPA receptors combined. A response can be any detectable signal including increases in calcium level, cAMP levels or transcription levels of a gene such as a reporter gene.

### G-Proteins

Typically a GPCR activates a G-protein to pass on a signal, but in some cases a GPCR is capable of signaling in the absence and/or without using a G-protein. In various embodiments of the invention, the GPCR couples with a specific G-protein and/or a promiscuous G-protein. In other embodiments, the GPCR does not couple with a G-protein. For example, various methods of the invention may be carried out wherein the GPCR signals by 1) activating a specific G-protein; 2) activating a promiscuous G-protein; 3) not activating a G-protein (e.g., activating through a pathway not involving a G-protein; or 4) any combinations thereof.

GPCR signaling is typically mediated by trimeric G-proteins containing alpha, beta, and gamma subunits and can be categorized into signaling classes based upon alpha subunit composition. G-alpha-q, G-alpha-s, and G-alpha-i/o proteins mediate intracellular signaling through activation of signaling pathways leading to distinct physiological endpoints. Activation of G-alpha-s and G-alpha-i/o coupled receptors typically leads to stimulation or inhibition of adenylate cyclase, respectively, while activation of G-alpha-q coupled receptors typically results in stimulation of phopholipase-C. GPCRs coupled to any of these types of G-proteins can be utilized in the present invention. GPCR signaling through these distinct pathways can be monitored by activation of specific transcriptional response elements placed upstream of a reporter coding region.

Certain G-proteins are considered “promiscuous” G-proteins because their G subunits allow them to couple with GPCRs that normally couple with G-proteins of other families (e.g., see PCT Publication No. WO 97/48820 and U.S. Pat. No. 6,004,808).

For example, two members of the G-alpha-q family, human G-alpha-16 and its murine homolog G-alpha-15, have been shown in transient cell-based systems to possess promiscuous receptor coupling. Although G-proteins having these G subunits are promiscuous with respect to the GPCR with which they couple, these G-proteins retain the ability to couple with a specific downstream effector. For example, regardless of which receptor is used to activate these G-proteins, the active promiscuous G subunit nonetheless activates Phospholipase(PLC)-C-beta. In one embodiment, a cell of the invention expresses or is engineered to express G-alpha-15 or G-alpha-16.

In some embodiments, a cell of the invention or a cell utilized in a method of the invention further comprises a non-native nucleic acid coding for a G-protein (e.g., G-alpha-15 or -16). For clarity, the G-protein itself may be a native or non-native protein. In one embodiment, the cell expresses native G-protein from a native nucleic acid. Controlling and/or upregulating expression of at least one G-protein, in will be beneficial for certain embodiments of the invention. For example, it may increase the sensitivity of a detection method. Additionally, sustained increased expression of a particular G-protein may be toxic to the cell. In one embodiment, a G-protein is expressed by a regulatable promoter.

In some embodiments of the invention, a cell additionally comprises a G-protein under control of a regulatable promoter. The G-protein can be under the same promoter sequence as the GPCR or a different promoter. In some cases, each is operatively linked to the same promoter. In some cases, the GPCR and G-protein coding regions are operatively linked, (e.g., via an IRES or self processing cleavage site), so as they are transcribed onto the same transcript. In other embodiments, they are expressed on different transcripts.

### Cells of the Invention

GPCRs, as well as other signaling pathway components, play important roles in cellular functions and also in many diseases and ailments. Therefore, the study of signaling pathway components (e.g., GPCRs), their functions and various GPCR modulators are of benefit and importance. In fact, as an example, there are enormous numbers of related GPCR experiments performed each year. GPCRs are involved in numerous types of cellular pathways. Many GPCRs still have not been linked to any function, cellular pathway and/or ligand. The cells and methods of the present invention can be utilized for this purpose.

One embodiment of the invention involves regulating expression of a GPCR, or other signaling pathway component, in a cell. In one embodiment, expression of a GPCR may be regulated by utilizing a nucleic acid comprising a regulatable promoter operatively linked to a GPCR coding region. Regulatable expression of a GPCR may be desirable for a number of reasons. For example, sustained and/or high level expression of a particular GPCR may be toxic to the cell (e.g., cause apoptosis). Further, it may also be desirable to study the effects of different expression levels for a GPCR. This may be combined with studying the effects of contacting a cell with at least one compound at different expression levels of a GPCR. In some cases, regulating expression of a GPCR may allow the development of a stable cell line that is capable of expressing a particular GPCR. In one embodiment, a stable cell line capable of expressing at least one GPCR is packaged in a kit. Of course, in instances where constitutive expression of a signaling pathway component (e.g., a GPCR) is not toxic to the cell in which it is expressed, a constitutive promoter may be used.

In one embodiment, a cell expresses an activated GPCR or other signaling pathway component. In one embodiment, a GPCR or other signaling pathway component is activated in the absence of a ligand. The inventors surprisingly discovered that when they expressed a GPCR from a regulatable promoter and induced expression of the GPCR, the GPCR was expressed in an activated form. Among other things, this characteristic allows screening for inhibitors of the activated GPCR pathway(s) in the absence of ligand. This is especially useful for orphan GPCRs.

In one embodiment, a parental cell does not express the GPCR or other signaling pathway component. In another embodiment, a parental cell expresses the GPCR or other signaling pathway component, e.g., the levels are too low for readily performing related assays. In another embodiment, a parental cell is modified or engineered to produce a higher level of the GPCR or other signaling pathway component than without modification.

Many cells can be used in the invention, e.g., for expression of a GPCR. Embodiments of the invention include, wherein the cell is selected from the group consisting of animal cells, plant cells, insect cells, yeast cells, human, murine and mammalian cells. Examples of cells that find use with the present invention include, but are not limited to, a 293 cell, a HEK cell, a HeLa cell, a Freestyle™ 293F cell (Invitrogen, California), a Per.C6 cell, a COS cell, a Vero cell, a mouse L cell, a 153DG44 cell, a human T-lymphocyte cell, baby hamster kidney (BHK) cells (e.g., ATCC No. CCL10), mouse L cells (e.g., ATCC No. CCLI.3), Jurkats (e.g., ATCC No. TIB 152) and 153 DG44 cells (e.g., see, Chasin (1986) Cell. Molec. Genet. 12: 555) human embryonic kidney (HEK) cells (e.g., ATCC No. CRL1573), Chinese hamster ovary (CHO) cells (e.g., ATCC Nos. CRL9618, CCL61, CRL9096), PC12 cells (e.g., ATCC No. CRL17.21) and COS-7 cells (e.g., ATCC No. CRL1651) or derivatives of any of these cells. In some embodiments, cells for heterologous cell surface protein expression are those that can be readily and efficiently transfected. In one embodiment, the cells are Jurkat cells, CHO cells or HEK 293 cells, such as those described in U.S. Pat. No. 5,024,939 and by Stillman et al. Mol. Cell. Biol. 5: 2051-2060 (1985). One skilled in the art can determine other cells that can be used with the invention.

In some instances, various GPCRs in a cell can be activated by the same ligands. Therefore, the ability to analyze a specific GPCR's activity or response to a ligand may be reduced in a cell expressing another GPCR(s) that responds to the same ligand. In these cases, the cell's response to the ligand may be due to the activity of both of theses GPCRs, possibly decreasing the sensitivity for detecting a response due to the GPCR of interest. However, cell of the invention may include more than one GPCR that is activated by the same ligand. In some of these embodiments, the sensitivity or assay window may be reduced as compared to a cell that does not express another GPCR activated by the same ligand. In some embodiments, a cell of the invention expresses a GPCR of interest that is activated by a ligand that does not activate another GPCR expressed in the cell. In some embodiments, a cell of the invention expresses an amount of a GPCR of interest that is activated by a ligand, wherein that ligand activates another GPCR expressed in the cell at a level of between from about 1.5 to about 3 fold, about 1.25 to about 2 fold, about 2 to about 100 fold, about 2 to about 3 fold, about 2 to about 4 fold, about 3 to about 5 fold, about 4 to about 6 fold, about 5 to about 7 fold, about 6 to about 8 fold, about 7 to about 9 fold, about 8 to about 10 fold, 2 to about 100 fold, about 2 to about 10 fold, about 2 to about 50 fold, about 5 to about 10 fold, about 5 to about 20 fold, about 10 to about 25 fold, about 25 to about 50 fold, or about 50 to about 100 fold less than the ligand activates the GPCR of interest.

In some embodiments, a cell of the invention is engineered to express a GPCR of interest and is engineered and/or selected to not express a GPCR which binds a particular ligand that activates the GPCR of interest. A cell population(s) can be screened for cells that do not express or have reduced expression of GPCR(s) that bind a particular ligand. These cells can be used as a parental cell to introduce a GPCR of interest as described herein. In some embodiments, a cell population(s) can be screened for cells that do not express or have reduced expression of GPCR(s) that bind a particular ligand as compared to the GPCR of interest.

Not wishing to be bound by theory, the inventors believe that at least for some GPCRs and/or cells, overexpression of the GPCR can cause expression of a constitutively activated population of a GPCR. This may be due to a percentage of an expressed GPCR being present in an activated state. When relatively high expression levels are obtained this population of activated GPCRs reaches an amount able to produce a detectable activation of the relevant pathway. When relatively low expression levels are maintained, the amount of activated GPCRs remains low and minimal or no detectable activation of the relevant pathway is detected.

As an example, a theoretical cell may require 100 activated molecules of a SPC (e.g., a GPCR) to result in a detectable increase in calcium levels. In the absence of a ligand, about 10% of the expressed SPC molecules may be in an activated state. Therefore, if the cell expresses 250 molecules of the SPC (e.g., expression of the SPC is uninduced and/or repressed) only 25 molecules will be in an activated state and there will be no detectable increase of calcium levels. In this case, the cells can be used for, inter alia, screening for agonists or screening for antagonists in the presence of a know ligand. In this case, if an agonist causes at least 75 more SPCs to reach an activated state then an increase in calcium levels would be detected. In another scenario, the cell expresses 1100 molecules of the SPC (e.g., expression of the SPC is induced and/or unrepressed) and therefore 110 molecules will be in an activated state resulting in a detectable increase of calcium levels. In this case, the cells can be used for, inter alia, screening for inverse agonists in the absence of a ligand.

Thus, the invention provides methods of making and using a cell that can be induced into an active state based on the expression of a GPCR(s) of interest. Inter alia, this type of cell line can be used to screen for inverse agonists. The invention provides methods wherein a cell can have the active state based on a particular GPCR(s) modulated. In some embodiments, expression of a GPCR in a cell can be reduced to a point where a population of the cells is not in an active state. These cells can be utilized to screen for and analyze, e.g., agonists that activate the GPCR, e.g., the GPCR population that is not in an activated state. In some embodiments, expression of a GPCR in a cell can be increased to a point where a population of the cells is in an active state relative to the GPCR pathway. These cells can be utilized to screen for and analyze, e.g., inverse agonists. In some embodiments, an inducible/repressible promoter is operatively linked to the GPCR's coding region in a cell to allow for modulating expression of the GPCR and possibly modulating the active state of the cell with regards to the GPCR's activation pathway. These cells can be utilized as described herein.

In some embodiments, a cell of the invention is capable of modulated expression of at least one SPC, e.g., a GPCR. In some embodiments, a cell of the invention can be modulated to have a difference in the expression levels of the SPC of between from about 1.5 to about 3 fold, about 1.25 to about 2 fold, about 2 to about 100 fold, about 2 to about 3 fold, about 2 to about 4 fold, about 3 to about 5 fold, about 4 to about 6 fold, about 5 to about 7 fold, about 6 to about 8 fold, about 7 to about 9 fold, about 8 to about 10 fold, 2 to about 100 fold, about 2 to about 10 fold, about 2 to about 50 fold, about 5 to about 10 fold, about 5 to about 20 fold, about 10 to about 25 fold, about 25 to about 50 fold, about 75 to about 100 fold or about 50 to about 100 fold. For example, the cell may express about 10 fold more of the SPC molecules when expression of the SPC of interest is induced as compared to uninduced.

In some embodiments, a cell of the invention can be modulated (e.g., via modulating expression of the SPC (e.g., a GPCR) of interest) to have a difference in the level of activation of a cellular pathway related to the SPC of interest of between from about 1.5 to about 3 fold, about 1.25 to about 2 fold, about 2 to about 100 fold, about 2 to about 3 fold, about 2 to about 4 fold, about 3 to about 5 fold, about 4 to about 6 fold, about 5 to about 7 fold, about 6 to about 8 fold, about 7 to about 9 fold, about 8 to about 10 fold, 2 to about 100 fold, about 2 to about 10 fold, about 2 to about 50 fold, about 5 to about 10 fold, about 5 to about 20 fold, about 10 to about 25 fold, about 25 to about 50 fold, about 75 to about 100 fold or about 50 to about 100 fold less than the ligand activates the SPC of interest. As an example, a cell of the invention may be capable of modulating the expression of a GPCR of interest (e.g., in the absence of a ligand) so as cAMP levels in the cell may increase about 10 fold more when expression of the GPCR of interest is induced as compared to uninduced.

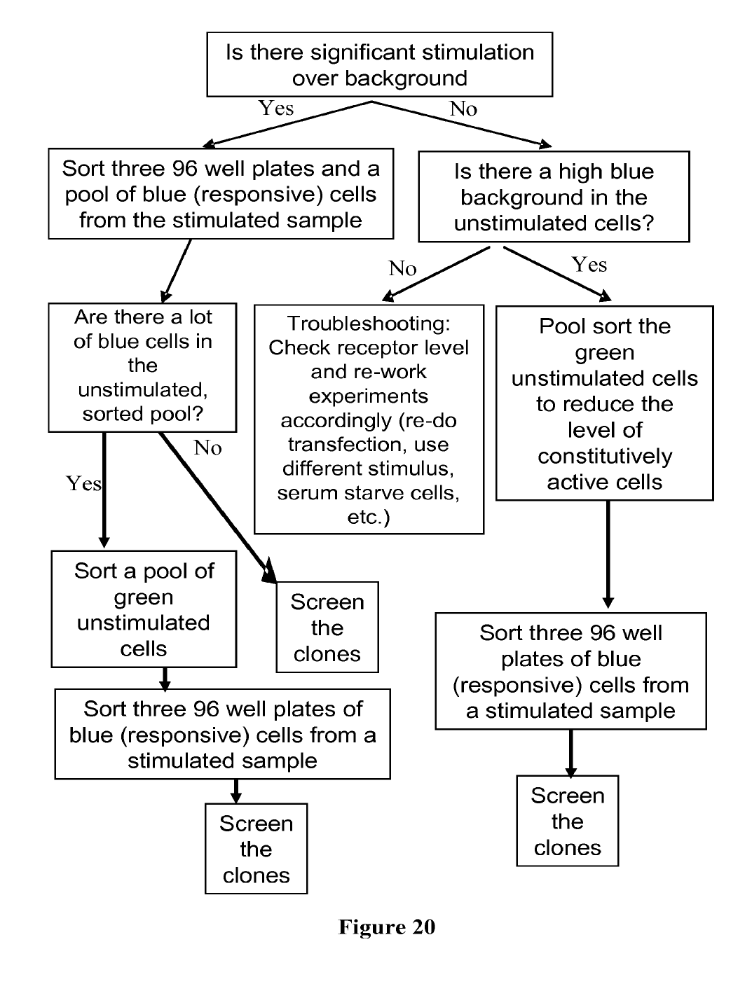

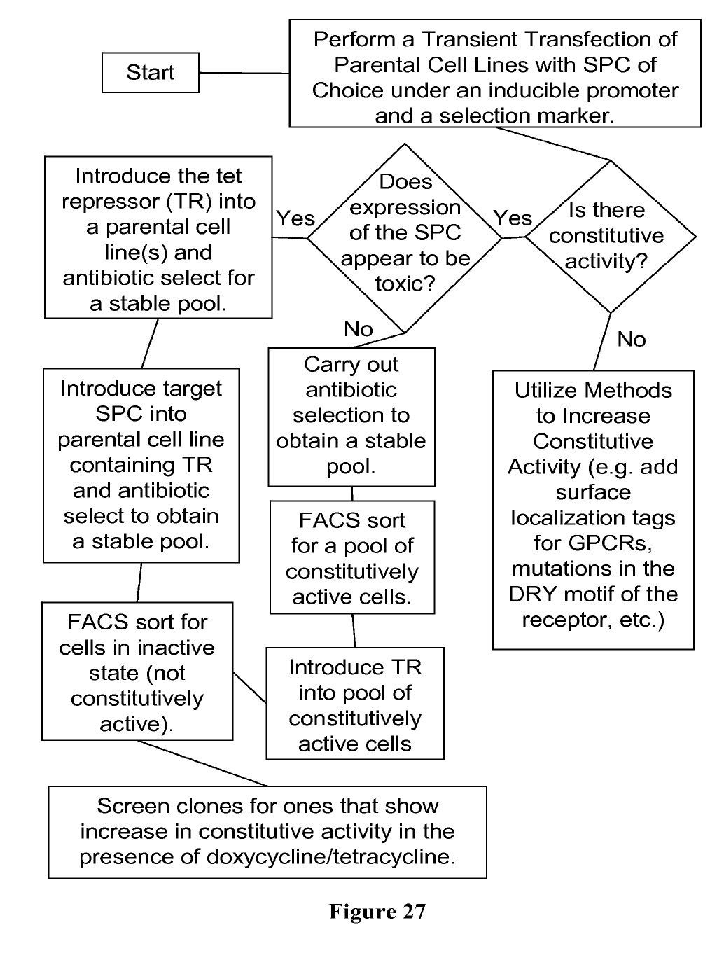

Another aspect of the invention relates to methods for the selection of stable cell lines and cell lines resulting from such selection. Stable cell lines can be functionally selected using a signal transduction detection system as described herein. The invention provides stable cells that are capable of expressing a GPCR or other signaling pathway component. In further embodiments, the cell line additionally is engineered to express a signal transduction coupling protein (e.g., a G-protein). FIGS. 20 and 27 are examples of flowcharts showing exemplary embodiments of the invention for selecting, producing or determining cells of the invention with desired characteristics. In some embodiments, the reporter polypeptide is beta-lactamase, but any compatible reporter polypeptide could be used.

One embodiment of the invention provides, a stable cell line expressing a G2A receptor that will couple to a G-protein signaling pathways (e.g., a pathway as described herein) and lead to a dose dependant, LPC stimulated increase in expression of beta-lactamase mediated by a specific response element located upstream of a beta-lactamase reporter coding region.

In some embodiments, the cell may also be engineered to express G-proteins capable of coupling with a GPCR or other signaling pathway component. Some embodiments of the invention utilize cells (e.g., NFAT-beta lactamase cell lines) containing a promiscuous G-protein which re-directs Gi/o coupled signaling to the Gq/NFAT pathway.

The cells of the invention can be employed in methods for (i) determining whether a polypeptide is a GPCR for a given ligand; (ii) determining whether a “test” ligand (e.g., a compound or antibody) is a ligand for a given GPCR; (iii) functionally characterizing the ability of a ligand to activate various GPCRs; and (iv) determining whether a compound modulates signal transduction in a cell (e.g., as an agonist or antagonist). The invention further includes similar methods where signaling pathway components other than GPCRs are used.

A “stable isolated cell” or “stable cell” of the invention is a cell that retains an expression construct typically longer than at least 3 to 4 passages in tissue culture. In other embodiments of the invention, a cell retains a construct longer than 6 to 7, 7 to 8, 9 to 10, 11 to 12, or longer than 12 passages. In some instances, the construct will be integrated into the genome of the host cells (e.g., into chromosomal DNA, mitochondrial DNA, etc.).

An “isolated” cell refers to a cell in an in vitro state (e.g., a cell of a mammalian tissue culture). In some aspects of the invention, the cell is an animal cell, a plant cell, a insect cell, a yeast cell, a human cell, a murine cell or a mammalian cell (e.g., a COS-7 cell).

One aspect of the invention includes a cell comprising a nucleic acid comprising a regulatable promoter operatively linked to a G-protein-coupled receptor (GPCR) coding region or a coding region of another signaling pathway component. In one embodiment the regulatable promoter is selected from the group consisting of a tetracycline inducible promoter, a T-REx™ promoter, heat shock inducible promoter, heavy metal ion inducible promoter, or nuclear hormone receptor inducible promoter or other promoter element whose activity is conditionally regulated. In one embodiment, the regulatable promoter comprises a tet operator. In another embodiment, the regulatable promoter comprises a CMV promoter element.

In some aspects of the invention, the GPCR or other signaling pathway component is expressed in an active form. In one embodiment, the GPCR or other signaling pathway component is expressed in an active form in the absence of its ligand. In one embodiment, the GPCR or other signaling pathway component is overexpressed in an active form in the absence of its cognate ligand.

In one embodiment, the cell further comprises at least one selectable marker. In some embodiments, the selectable marker(s) and GPCR or other signaling pathway component coding region are on the same nucleic acid. In some embodiments, the GPCR or other signaling pathway component and the selectable marker coding regions are operatively linked with an IRES or self processing cleavage site. In other embodiments, the GPCR or other signaling pathway component and selectable marker coding regions are operatively linked to different promoters. In one embodiment, the selectable marker and GPCR or other signaling pathway component coding region are on different nucleic acids. In one embodiment, the GPCR or other signaling pathway component coding region is from a cDNA.

The present invention provides numerous nucleic acids, e.g., those encoding a GPCR(s), a selectable marker(s), a reporter polypeptide, a G-protein or any combination thereof. In some embodiments, a nucleic acid is a DNA or RNA. In some embodiments, the nucleic acid is a plasmid, a viral vector, a synthetic microchromosome or composes a transposons. Examples of viral vectors include, but are not limited to, a baculovirus derivative, an adenovirus, an Adeno-associated virus, a lentivirus, a retrovirus, or other viral vectors for delivery of genes into cells. In some embodiments, the SPC encoding nucleic acid is integrated into a cell's chromosome at least once, but there may be multiple integrants.

In one embodiment, the cell further comprises an intracellular calcium indicator.

In some aspects of the invention, a cell of the invention comprises a GPCR coding region for a Class A GPCR, a Class B GPCR, a Class C GPCR, a Class F/S GPCR, an orphan GPCR or non-orphan GPCR. In one embodiment, the GPCR coding region codes for a G2A GPCR.

In some aspects of the invention, the cell further comprises a nucleic acid comprising a second promoter operatively linked to a coding region for a reporter polypeptide. In one embodiment, the regulatable promoter operatively linked to a GPCR coding region and the second promoter operatively linked to a coding region for a reporter polypeptide are on the same nucleic acid. In some embodiments of the invention, the regulatable promoter operatively linked to a GPCR coding region is on a nucleic acid different than the second promoter operatively linked to a coding region for a reporter polypeptide. In one aspect of the invention, the regulatable promoter is operatively linked to a GPCR coding region pre-existing in the genome of the cell. In various aspects of the invention, the second promoter is regulated directly or indirectly by the amount of activated GPCR. In one embodiment, the second promoter is regulated by the amount of or change in the amount of intracellular calcium. In one aspect the second promoter is regulated by the amount of or change in the amount of intracellular cAMP. In some aspects of the invention, the second promoter comprises a calcium responsive element. In one embodiment, the second promoter comprises a cAMP responsive element. Embodiments of the invention include, wherein the second promoter comprises a responsive element selected from the group consisting of an NFAT responsive element, a cAMP responsive element (CRE) and a kinase C-responsive promoter.

In one embodiment, the NFAT responsive element comprises at least one copy of the nucleotide sequence GGAGGAAAAACTGTTTCATACAGAAAGGCGT (SEQ ID NO:1) or GGAAAAACTGTTTCA (SEQ ID NO:7). In some embodiments, the promoter comprises 2, 3 or more than 3 copies of SEQ ID NO:1 and/or SEQ ID NO:7. For clarity the NFAT responsive element (e.g., SEQ ID NO:1 and/or SEQ ID NO:7) can be in any orientation. In one embodiment, a calcium responsive promoter comprises 3 copies of SEQ ID NO:1 or 7, e.g., wherein one copy is in the forward orientation and 2 copies are in the reverse orientation with respect to the coding region.

In one embodiment, the second promoter is regulated by the amount of or change in the amount of cAMP amounts. In one embodiment, a cAMP responsive element comprises the nucleotide sequence of CGACGTCA (SEQ ID NO:2) or TGACGTCA (SEQ ID NO:8). In each of the above instances, a signaling pathway component other than a GPCR may be used.

In one embodiment, the cell further comprises a nucleic acid encoding a polypeptide having a biological activity of a promiscuous G-alpha protein. In some embodiments, the second promoter of the reporter polypeptide is indirectly modulated by the activity of a promiscuous Gα15 protein, chimeric G proteins, Gqi5, or Gqo5.

In some embodiments, the expressed GPCR is coupled to either G-alpha-i, G-alpha-s or G-alpha-12 in the absence of a G-alpha-15 protein. In some aspects of the invention, the GPCR is coupled to at least one G-protein selected from the group consisting of Gi, Go, Gs, Gq, Ga12/13, Galpha15, G alpha16, chimeric G proteins, Gqi5, or Gqo5.

In some embodiments of the invention, the reporter polypeptide is detected directly or indirectly by fluorescence, light absorption, colorimetric readout, detecting an enzyme reaction, immunohistochemistry, immunofluorescence, flow cytometry, fluorescent-activated cell sorting (FACS), luminescence or FRET. The reporter polypeptide may be, but is not limited to, a beta-lactamase, a fluorescent polypeptide, a luciferase, a green fluorescent protein (GFP), a chloramphenicol acetyl transferase, an alkaline phosphatase a galactosidase, an alkaline phosphatase, and a human growth hormone.

In one embodiment, the expression of the reporter polypeptide is increased when the amount of activated GPCR is increased. In one embodiment, the expression of the reporter polypeptide is decreased when the amount of activated GPCR is increased. For example, the reporter polypeptide coding region is operatively linked to a promoter that is repressed directly or indirectly by the GPCR activation. In one embodiment, the reporter polypeptide is increased when the amount of activated GPCR is decreased. In another aspect of the invention, expression of the reporter polypeptide is decreased when the amount of activated GPCR is decreased.

In one embodiment, the cell does not express a G-alpha subunit. In one embodiment, the cell does not express a promiscuous G-protein (e.g., G-alpha-15 or G-alpha-16). In another embodiment, the cell is not engineered to express a G-alpha subunit. In one embodiment, the cell is not engineered to express a promiscuous G-protein (e.g., G-alpha-15 or G-alpha-16).

### Methods for Constructing Cells of the Invention

As discussed above, the invention provides, inter alia, stable cell lines that are capable of expressing an SPC. In some embodiments, the SPC (e.g., a GPCR, a kinase, a nuclear receptor, an ion channel or a G-protein) is expressed in an active state. In some embodiments, expression of a GPCR is controlled by a regulatable promoter and the cell is capable of expressing the GPCR in an active state, even in the absence of its respective ligand.

Utilizing the teachings herein, one skilled in the art is able to use numerous methods to construct cells of the invention or practice methods of the invention. The following methods for constructing the cells of the invention are provided as examples and are not meant to limit methods of the invention.

The invention provides various methods as described herein for constructing or producing the cells of the invention. In one embodiment, a GPCR receptor is transfected into a variety of parental cell lines (e.g., HEK, CHO, or Jurkat) or just one parental cell type. In some embodiments, the parental cell contains a reporter coding region that is operatively linked to a promoter that is modulated (directly or indirectly) by an active GPCR (e.g., an NFAT response element and/or a cAMP response element (CRE) operatively linked to a beta-lactamase reporter coding region). In some embodiments, the transfected cells are then stimulated with a ligand (e.g., LPC in the case of G2A) to determine if an agonist induced response can be detected. In one embodiment, the GPCR is already expressed in an active state making the addition of its ligand unnecessary. Variations of the above may be employed in which the signaling pathway component is something other than a GPCR.

In one embodiment, a GPCR is introduced (e.g. transfected) into multiple cell lines, each containing a reporter coding region that is operatively linked to a promoter that is modulated (directly or indirectly) by an active GPCR (e.g., a NFAT response element and/or a cAMP response element (CRE) operatively linked to a beta-lactamase reporter coding region). The multiple cell lines can vary in, the reporter coding region, the promoter driving the reporter, the cell type, transfection or infection methods, or combinations thereof. Then the cells can be screened for the desired function and the cells that best fit the planned method may be utilized. For example, one will be able to determine which cellular background is most suitable for this assay and which coupling pathway is most likely to lead to a functional GPCR assay. In one embodiment, a GPCR is introduced into only one cell line or parental cell type. Again, variations of the above may be employed in which the signaling pathway component is something other than a GPCR.

In some embodiments, the invention provides methods of expressing a constitutively activated SPC in a cell comprising introducing into a population of cells a nucleic acid comprising a regulatable promoter operatively linked to a SPC (e.g., a GPCR) coding region and culturing the cell under conditions wherein the SPC is expressed.

The invention also provides methods for constructing a stable cell capable of expressing an activated SPC (e.g., a GPCR, a kinase, a nuclear receptor, an ion channel or a G-protein). In another embodiment, the methods comprise introducing into a first population of cells a nucleic acid comprising a regulatable promoter operatively linked to a SPC coding region and sorting of the first population, wherein the cells have been cultured under conditions to minimize expression of the SPC and the cells are sorted for cells that have no or low expression levels of the SPC to create a second population of cells. For example the cells are cultured so as not to induce expression from a regulatable promoter or even repress expression from the regulatable promoter. Then the cells are sorted to remove cells that are still expressing or expressing high levels of the SPC and cells with little or no expression of the GPCR are isolated.

In some embodiments, the isolated cells are then cultured under conditions to activate, induce and/or derepress the regulatable promoter allowing expression of the GPCR. The cells may then be sorted and those expressing desirable levels of GPCR are isolated. In some embodiments, the cells are sorted for those expressing an activated GPCR.

Some embodiments therefore include a first round of sorting to eliminate cells that are expressing the GPCR at undesirable levels without activation or derepression of the promoter. In some embodiments, a second sort then selects for cells that express a GPCR (e.g., activated) in response to activation of the promoter. The second sort provides cells that not only express the GPCR, but express an activated form or express a higher amount or percentage of the activated GPCR.

The cell sorts/selections can be carried out using various methods known in the art. The cells may be sorted using an antibody that binds the GPCR on the cell surface, e.g., FACS or beads attached to this antibody. As discussed above, the cells can also be sorted for cells that express an activated form or express a higher amount or percentage of the activated GPCR. This can be accomplished utilizing various techniques. For example, see Examples 3 and 4 below. In some embodiments the cells contain a signaling pathway promoter (SPP) operatively linked to a reporter polypeptide coding region, e.g., wherein upon expression of an activated GPCR the SPP upregulates expression of the reporter. In one embodiment, the reporter is a beta-lactamase reporter. This allows the cells to be sorted by FACS. The reporter polypeptide construct can be introduced into the cells prior to, simultaneously with, or after introducing the nucleic acid encoding the SPC (e.g., GPCR).

Therefore, the invention provides methods comprising introducing into a first population of cells a nucleic acid comprising a regulatable promoter operatively linked to a SPC coding region and sorting of the first population, wherein the cells have been cultured under conditions to minimize expression of the SPC and the cells are sorted for cells that have no or low expression levels of the SPC to create a second population of cells. The second population of cells may optionally be cultured under conditions to express or maximize expression of the SPC and the cells are sorted for cells that express the SPC in an activated state to create a third population of cells. This third population of cells may be utilized in the assays and methods of the invention or optionally for isolating clonal populations of cells. The clones may be used in methods of the invention. The cloned cells may be further characterized.

The nucleic acids of the invention may essentially be introduced into a cell by any known methods, e.g., by transfection, electroporation, microinjection, or infection with a viral vector. In some embodiments, the second promoter (e.g., SPP) operatively linked to the reporter coding region regulates expression by the amount of or change in intracellular calcium amounts. In one embodiment, the second promoter comprises at least one responsive element selected from the group consisting of an NFAT responsive element, a cAMP responsive element (CRE) and kinase C-responsive promoter.

In some embodiments, expression of the reporter polypeptide is increased when the amount of activated GPCR is increased; is decreased when the amount of activated GPCR is increased; is increased when the amount of activated GPCR is decreased; or is decreased when the amount of activated GPCR is decreased.

In some embodiments the SPC (e.g., a GPCR, a kinase, a nuclear receptor, an ion channel or a G-protein) is toxic to the cell or inhibits the establishment of a stable cell line when constitutively expressed.

In some embodiments, a cell is further engineered to express a second GPCR. The second GPCR may be constitutively expressed or also controlled by a regulatable promoter.

### GPCR Libraries

As discussed above, the present invention further provides cells expressing a GPCR library. In one embodiment, the GPCR library is comprised of a variety of wild-type GPCRs. In another embodiment, the GPCR library is comprised of mutants of at least one GPCR.

For example a library of GPCRs can be constructed by creating a mutant library of a particular GPCR. Methods of creating mutant libraries are well known in the art. The library can be composed of any number of mutants, e.g., including, but not limited to, 2, 5, 10, 50, 100, 500, 1000, 10⁴, 10⁵, 10⁶, 10⁷, 10⁸, 10⁹, any numbering between or even more than 10⁹ mutants. The library of mutant GPCRs is then cloned in to a cell of the invention. One embodiment of the invention includes cells comprising a library of wild-type GPCRs. In one embodiment, cells of the library comprise a nucleic acid comprising a promoter operatively linked to a G-protein-coupled receptor (GPCR) coding region.

The library of GPCR expressing cells can then be screened for GPCRs with a desired function, e.g., activation, inhibit or decrease activation, or essentially cause no change in activation of the GPCR by a certain ligand. In one embodiment, the library is screened for GPCRs for which a certain ligand is an agonist or an antagonist or an inverse agonist. In another embodiment, the library is screened for cells that have a certain characteristic (e.g., a change in cAMP levels, apoptosis and/or a change in intracellular calcium levels). In one embodiment, the cells are screened based on fluorescence. In one embodiment, the cells are screened utilizing cell sorting, e.g., FACS. In some embodiments, a library of GPCR expressing cells comprises a reporter coding region that is operatively linked to a promoter that is modulated (directly or indirectly) by an active GPCR (e.g., a NFAT response element and/or a cAMP response element (CRE) operatively linked to a beta-lactamase reporter coding region).

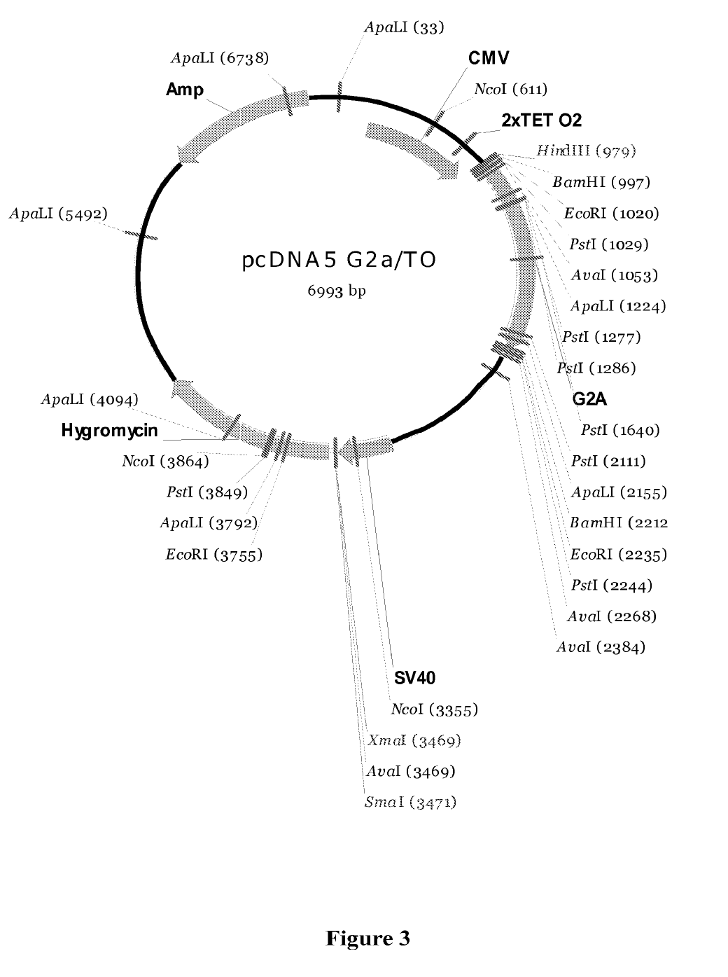

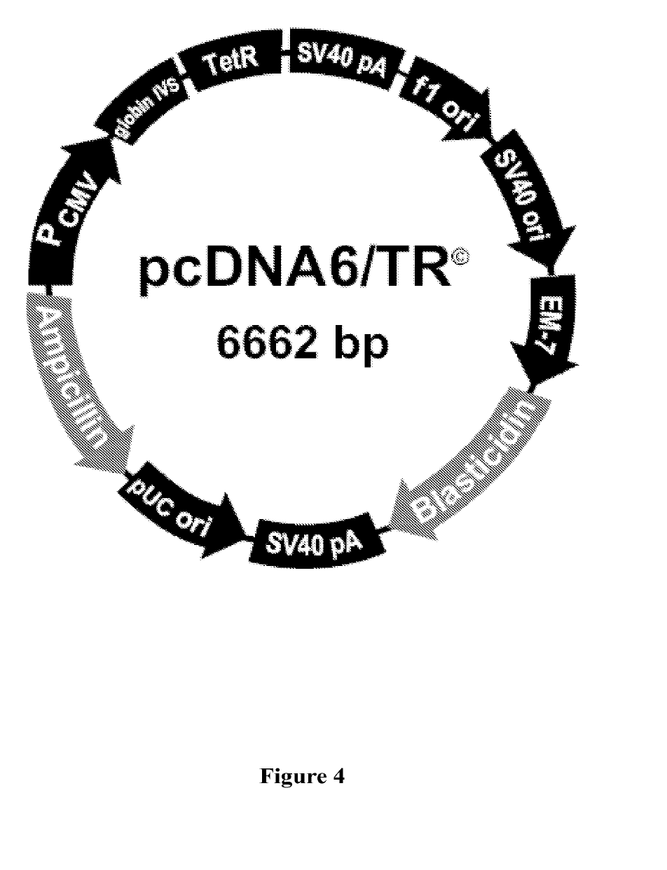

In many instances, libraries of the invention will contain nucleic acids which are introduced into vectors which replicate in eukaryotic cells (e.g., animal cells such as mammalian cells). Examples of vector backbones which may be used to construct libraries of the invention are shown in FIGS. 3 and 4.

### Nucleic Acids, Promoters and Constructs of the Invention

The amount of GPCR expressed in a cell can be titrated by using, or selecting for, either a weak promoter, strong promoter, a regulatable promoter (e.g., an inducible promoter) or selecting a population of cells for the desired expression characteristics. An inducible promoter can offer the advantage of regulatable expression of a GPCR. By using an inducible promoter the amount of inducer can be used to optimize the signal to noise ratio of, for example, a screen for GPCR modulators by adjusting the amount of GPCR expression from the cell.

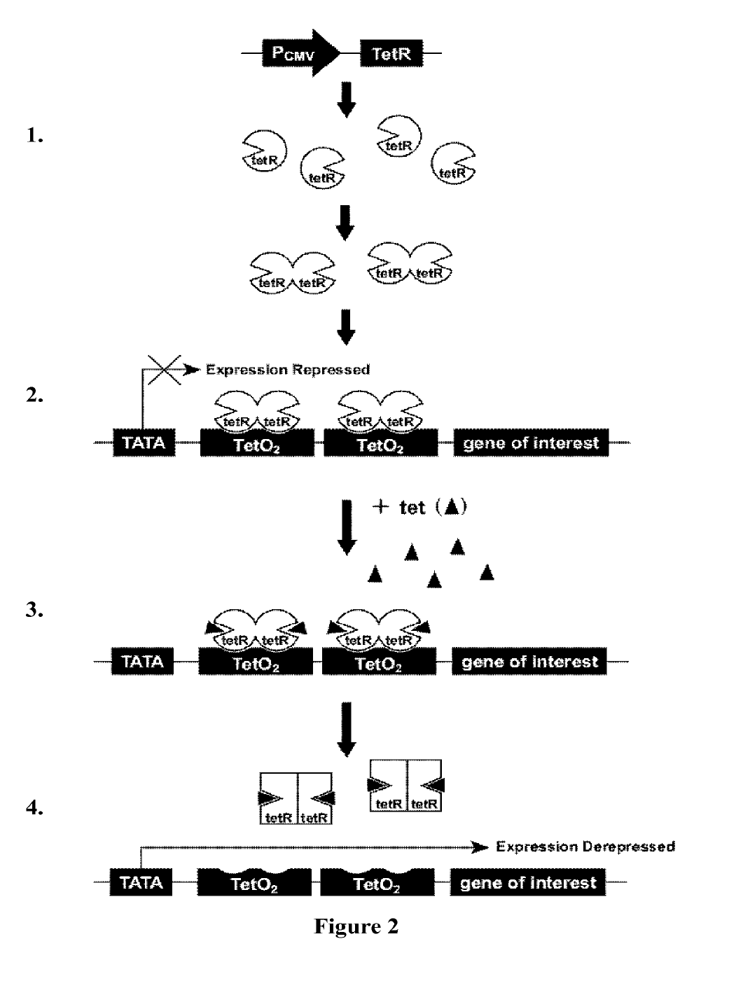

“Regulatable” promoters are promoters which one can modulate their transcriptional activity. For example, the level of expression or transcription can be modulated by introduction of an agent (e.g., tetracycline) or an environmental condition (e.g., heat inducible). Thus, regulatable promoters include inducible and repressible promoters. An inducible promoter is one which can be activated by the addition of an agent. A repressible promoter is one which exhibits decreased transcriptional activation activity in the presence of a repressor. In some instances, a promoter can be both inducible and repressible. For example, promoters can be constructed so that a protein binds and represses them except for when an inducer is present (see FIG. 2). Examples of agents that can regulate promoters are compounds including, but not limited to, tetracycline or doxycycline, transcription factors, DNA binding proteins, hormones, drugs, etc. and changes in environmental factors, such as e.g., temperature change (e.g., heat inducible promoters), oxygen level change, radiation, etc. In some embodiments, in the absence of an inducer (e.g., doxycycline) the promoter does not direct expression, or directs low levels of expression (e.g., produces less than 500 proteins per cell at steady state) of an operatively linked coding region (including cDNA). In another embodiment, in the presence of an inducer, the expression of the polypeptide (e.g., a GPCR) directed by the inducible/regulatable promoter is typically increased at least 3-, at least 10-, at least 100-, or at least 1,000-fold. Other useful regulatable promoters include those that are inducible by IPTG or ecdysone. If desired, a regulatable promoter can include a first promoter (e.g., a cytomegalovirus promoter) operatively linked to a tet operator to regulate the first promoter (e.g., see, Gossen and Bujard, 1992, Proc. Natl. Acad. Sci. 89:5547-5551). In some embodiments, a regulatable promoter is repressed in the presence of an agent and is activated in low concentration or in the absence of the agent.

In one embodiment, a regulatable promoter used to control the expression of a GPCR or other signaling pathway component is not the native promoter normally associated with the coding regions. By non-native promoter is meant that the sequence of the promoter is not the same as the native promoter for that particular GPCR in the cell. Examples of non-native promoters include, but are not limited to, a native GPCR promoter sequence that has been mutated. Examples of mutated promoter sequences include a GPCR promoter that has been shortened, contains a deletion, insertion and/or substitution, and/or a GPCR promoter operatively linked to a GPCR coding region that is not the promoter's native GPCR coding region.

In one embodiment, a cell of the invention can contain a polynucleotide having a control sequence and encoding a protein useful in a signal transduction detection system. Such a construct may be designed, for example, to detect activation of a GPCR or other signaling pathway component. In one embodiment, this construct is typically located on a second vector. It can include a reporter coding region that is operatively linked to a promoter that is modulated (directly or indirectly) by an active GPCR or other signaling pathway component. In one embodiment, the expression of the reporter polypeptide can be detected by detecting a change in fluorescence emission of a sample that contains the cell.

In some embodiments, Stratagene's Complete Control™ Inducible Mammalian Expression System (e.g., Stratagene, La Jolla, Calif., cat. nos. 217460, 217461, and 217468), which involves a synthetic ecdysone-inducible receptor, or its pET Expression System can be used in practicing the present invention. In some embodiments, an inducible expression system available from Invitrogen Corp. (Carlsbad, Calif.), which carries the T-REX™ (tetracycline-regulated expression) System (see, e.g., cat. no. V1033-20), which employs an inducible mammalian expression system that uses the full-length CMV promoter, can be used to practice the invention. Invitrogen also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### Reporter Polypeptides

In one embodiment, the reporter polypeptide is similar to or as those described in Tsien et al., U.S. Pat. Nos. 5,741,657 and 6,291,162, the entire disclosures of which are incorporated herein by reference. These reporter polypeptides allow detection and isolation of both expressing and non-expressing single living cells.

The assay system described in U.S. Pat. Nos. 5,741,657 and 6,291,162 uses a non-toxic, non-polar fluorescent substrate, which is easily loaded and then trapped intracellularly. Cleavage of the fluorescent substrate by beta-lactamase yields a fluorescent emission shift as substrate is converted to product. In one embodiment, a beta-lactamase reporter readout is ratiometric. A beta-lactamase polypeptide reporter system allows the control of variables such as the amount of substrate loaded into individual cells. Beta-lactamase is stable, easily detected with an intracellular readout that can simplify assay procedures by eliminating the need for washing steps. These features can facilitate screening with cells using the invention. In one embodiment, a ratiometric fluorescent signal transduction detection system can be used with the invention. The present invention includes, but is not limited to, fluorogenic substrates such as described in U.S. Pat. Nos. 5,741,657 and 6,291,162.

In one embodiment, the reporter system is the GeneBLAzer® beta-lactamase reporter system (Invitrogen, Carlsbad, Calif.). In one embodiment, a nucleic acid encoding for the reporter polypeptide is comprised of a beta-lactamase coding region, e.g., from the GeneBLAzer®, LiveBLAzer™ and LyticBLAzer™ constructs sold by Invitrogen (Carlsbad, Calif.). The GeneBLAzer® cell-based beta-lactamase (bla) reporter assay system combines molecular and cell biology with a Fluorescence Resonance Energy Transfer (FRET)-based detection method to create flexible, sensitive, high-throughput screening (HTS) assays for drug discovery in single live cells. GeneBLAzer® Technology, can be used to probe the biological activity of a protein or a pathway inside the cell or in cell lysates. The GeneBLAzer® cell-based assay system is suitable for studying numerous target classes and cellular processes. GeneBLAzer® Technology uses a mammalian-optimized gene, bla, combined with a FRET-enabled substrate to provide sensitive detection in live cells. Cells are loaded with fluorescent substrate (e.g., CCF2 or CCF4). In some instances, these substrates contain the two fluorophores coumarin and fluorescein. In the absence of bla expression, the substrate molecule remains intact. Excitation of the coumarin results in fluorescence resonant energy transfer to the fluorescein moiety. Using CCF2 as an example, this energy transfer causes the fluorescein to emit green light with an emission peak of about 520 nm. However, in the presence of bla expression, fluorescent substrate is cleaved, separating the fluorophores, and disrupting the energy transfer. Again using CCF2 as an example, excitation of the coumarin in the presence of enzyme activity results in a blue fluorescence signal at about 447 nm. In a population of cells loaded with CCF2 substrate, those that fluoresce blue contain beta-lactamase activity and those that fluoresce green do not. Further, this system allows for quantitation of beta-lactamase activity based upon, for example, fluorescent intensities generated by uncleaved and cleaved fluorescent substrate.

Some embodiments of the invention utilize GeneBLAzer® Master Cell Lines with NFAT or CRE response elements including, but not limited to, Jurkat, CHO-K1, or Freestyle™ 293F cell backgrounds for development of assays in suspension or adherent cell format. GeneBLAzer® cell lines from Invitrogen are provided with various protocols including transfection procedures that may be utilized with methods of the current invention.

The NFAT response element is sensitive to signaling pathways which lead to a rise in intracellular calcium. When intracellular calcium levels rise, calmodulin, a calcium ion sensitive subunit of phosphorylase kinase, is activated. Phosphorylase kinase in turn phosphorylates the phosphatase, calcineurin. Calcineurin dephosphorylates cytoplasmic NFAT which translocates to the nucleus and induces transcription of NFAT-responsive genes.

The cAMP response element is sensitive to a change in intracellular cAMP levels. When adenylate cyclase is activated through GPCR signaling, ATP is converted to cAMP. The cAMP in turn activates a cAMP-dependant protein kinase which in turn phosphorylates and activates cAMP response element binding protein (CREB). CREB in turn induces transcription of CRE-responsive genes.

Other reporter polypeptides having the biological activity of green fluorescent protein (GFP) can be used. In one embodiment, the reporter polypeptide is a fluorescent polypeptide. In another embodiment, the reporter polypeptide is a GFP or fluorescent fragment.

One strategy for addressing potentially high background levels of beta-lactamase activity, e.g., arising from constitutive signaling by the GPCR (e.g., G2A) is to treat (e.g., pre-treat) cells with a beta-lactamase inhibitor, e.g., clavulanic acid. This could be done before running the assay. By pre-treating cells with clavulanic acid, the background level of beta-lactamase activity would be significantly reduced. Prior to stimulation of cells, the clavulanic acid could then be removed from both the unstimulated and stimulated samples. In certain embodiments, this lowering of the initial level of beta-lactamase activity present (e.g., from the constitutive signaling) will allow agonist induced stimulation to be detected over background.

Alternative signaling pathways may be considered. Examples of alternative pathways are the NF-kB signaling pathway, used to detect signaling from the Gα13 coupled pathway, or a cell line with a serum response element (SRE) linked to reporter polypeptide coding region (e.g., beta-lactamase) to detect signaling from the Gi coupled pathway acting through the Ras/Raf pathway and the Elk-1 transcription factor.

### Methods of the Invention

The invention includes, but is not limited to, methods 1) of identifying a ligand for a GPCR or other signaling pathway component; 2) of identifying a GPCR or other signaling pathway component for a given ligand; 3) of identifying a modulator of a GPCR or other signaling pathway component; and 4) of expressing a GPCR (e.g., in a constitutively active state). In some embodiments, the method comprises: a) contacting a cell of the invention with a test compound and b) detecting a signal resulting from expression of a reporter polypeptide. Methods of the present invention may be carried out, for example, with a cell of the invention capable of expressing a putative GPCR, a GPCR of known function or a GPCR of unknown function in a cell. Some methods of the invention comprise contacting a cell of the invention with a test compound and/or a ligand known to be a ligand for the GPCR. Some aspects of the invention comprise detecting a calcium level within the cell. GPCRs are known to, inter alia, affect calcium and/or cAMP levels in a cell. Calcium ions are typically produced in cells upon activation of GPCRs (e.g., coupled to Gq-proteins of the three main families of G-proteins). Even though intracellular calcium levels typically rise directly from Gq-protein receptor activation, genetic expression methods have been developed that allow calcium ion production to proceed upon activation of GPCRs coupled to other G protein types (e.g., Gi/Go or Gs). Fluorescent calcium screening can be used in an approach for screening compounds against GPCRs.

In one embodiment, the present invention includes a cell expressing a GPCR under the control of a regulatable promoter. The invention further provides methods related to expressing a GPCR or other signaling pathway component under the control of a regulatable promoter in a cell and monitoring and/or detecting intracellular calcium levels and/or changes of intracellular calcium levels. Such methods may be performed in a cell in the absence or presence of a reporter coding region operatively linked to a promoter regulated directly or indirectly by the amount of activated GPCR.

Activation of G-alpha-s and G-alpha-i/o coupled receptors typically leads to stimulation or inhibition of adenylate cyclase, respectively, while activation of G-alpha-q coupled receptors typically results in stimulation of phopholipase-C. GPCRs coupled to any of these types of G-proteins can be utilized in the present invention. GPCR signaling through these distinct pathways can be monitored, e.g., by activation of specific transcriptional response elements placed upstream of a reporter polypeptide coding region.

In some methods of the invention, the readout does not involve transcription/expression of a reporter polypeptide. Numerous methods of detecting cellular changes caused by a GPCR are know in the art. These methods may be used in combination with a reporter system as described herein or can be used alone or in the absence of a reporter system. Changes in intracellular cAMP and/or calcium are just two examples of changes that can be a modulated by GPCR activation or lack thereof. Both of the changes can be measured by methods (known in the art) that do not involve transgene expression.

Numerous methods of measuring increases of intracellular calcium are known in the art. In one embodiment, the assay is a direct calcium readout, e.g., using Fluo4 (Molecular Probes, Eugene, Oreg., an Invitrogen company). Another example of a commercially available product which can be used to measure increases of intracellular calcium is AequoScreen (Euroscreen, Brussels), which is based on a jellyfish-derived photoprotein called aequorin that displays photoactivity proportional to calcium ion concentration. Screening a library against an array of GPCR expressing cells (e.g., GPCR-overexpressing cells) mixed with aequorin provides a quantitative means of assessing a compound's ability to activate a GPCR (or its ability to antagonize activation). Other methods for detecting and/or measuring calcium levels include the use of Fura-2, Fura-red, Rhod-2 (e.g., catalog# R14220, R1245MP or R1244, Invitrogen), X-Rhod-1 (e.g., catalog# X14209 or X14210, Invitrogen), Rhod-5N, Rhod-FF, X-Rhod-5F, X-Rhod-FF, Fluo calcium indicators e.g., (Fluo-3, Fluo-4, Fluo-4FF, Fluo-5F, Fluo-5N, or Mag-Fluo-4, all available from Molecular Probes, Eugene, Oreg., an Invitrogen company), Calcium Green™, Calcium Yellow™, and Calcium Crimson™ (e.g., available from Invitrogen, Carlsbad, Calif.)

Numerous methods of measuring increases of cAMP are known in the art. Cyclic adenosine monophosphate (cAMP) is an example of a “second messenger” compound in the GPCR activation process. In one embodiment, cAMP is used as a high-throughput screening marker. In one embodiment, detection of cAMP is accomplished using luminescent tags that bind to cAMP. In one embodiment, changes in cAMP are determined using a LANCE assay (Perkin Elmer). In one embodiment, Melanophore technology (Arena Pharmaceuticals) is used to detect changing levels of cAMP. Melanophore technology involves expressing GPCR targets in frog skin cells containing a pigment that is highly sensitive to changing levels of cAMP. In this system an increase of intracellular levels of cAMP results in the pigment being dispersed throughout the cell and appears black. If there is a decrease in the level of cAMP, the pigment aggregates to the center and the cell appears “clear.” BD ACTOne™ is a cAMP biosensor and is another method for measuring cAMP levels that may be used with the present invention.

In one embodiment, the GPCR is capable of coupling with or is coupled to a Gs- and/or Gq-protein. Activation of a Gs- and/or Gq-protein typically stimulates cAMP production, whereas typically Gi/Go-coupled receptors inhibit cAMP. In one embodiment, the GPCR is capable of coupling with or is coupled to Gi/Go-proteins. Therefore, methods of the invention can involve detecting increases or decreases in the cAMP levels of the cell.

In some aspects of the invention, changes in or the state of activation of GPCRs can be assayed using a Tango assay. In some embodiments, a target GPCR is fused at its intracellular C-terminus to an exogenous transcription factor. Interposed between the receptor and the transcription factor is a specific cleavage sequence for a protease (e.g., non-native). This chimeric receptor protein is expressed in a cell line containing a reporter gene responsive to the transcription factor. In these aspects of the invention the chimeric receptor protein may be expressed from a regulatable promoter. In some aspects, the chimeric receptor protein expressed from the regulatable promoter is expressed in an active state in the absence of a ligand that activates the receptor. A similar method of measuring GPCR activation is described in U.S. patent publication US20050100934.

In other embodiments, changes exerted by a GPCR or other signaling pathway component are monitored or detected utilizing a reporter coding region operatively linked to a promoter that is responsive to a cellular change mediate directly or indirectly by the GPCR (e.g., changes in calcium and/or cAMP levels) or other signaling pathway component. Examples of response elements that may be included in the promoter that are responsive to a cellular change are a NF-kB response element, a NFAT response element and a cAMP responsive element. In these embodiments, a change in the activation state of a GPCR can be measured by the change in expression from the reporter coding sequence.

In one embodiment, a compound is evaluated against a cell of the invention expressing a GPCR and, as a control, the compound is evaluated against a cell of the invention not expressing or with a decreased expression of the same GPCR. In certain instances, an effect is seen in the GPCR expressing cell, but not in the cell with decreased expression of the same GPCR suggests the effect is caused by a direct or indirect interaction between the GPCR and the compound. In one embodiment, the cell with decreased expression of the GPCR lacks a coding region for the GPCR. In another embodiment, the GPCR coding region is operatively linked to a regulatable promoter. In this case, the culture conditions of one cell population causes an increased expression level over the culture conditions of another cell population. Using a tetracycline responsive promoter operatively linked to the GPCR as an example, one population of cells is cultured in the presence of tetracycline and the other population is grown in the absence or with a lower concentration of tetracycline. As one skilled in the art would understand, methods described above could employ a signaling pathway component other than a GPCR.

In some embodiments of the invention, expression of the reporter polypeptide can be modulated (e.g., expression level can be increased) through a G-protein signaling pathway. In one embodiment, PLC-beta is activated. In one embodiment, activation of the GPCR increases intracellular calcium levels. In one embodiment, activation of the GPCR decreases intracellular calcium levels. In one embodiment, an increase in calcium levels can lead to modulation of a “calcium-responsive” promoter that is, for example, part of a signal transduction detection system, e.g., a promoter that is activated (e.g., a NFAT promoter) or inhibited by a change in calcium levels. In one embodiment, an NFAT DNA binding site is as described, similar or derived from the NFAT DNA binding site described in Shaw, et al. Science 291:202-205 1988. In one embodiment, a promoter that is responsive to changes in protein kinase C levels (i.e., a “protein kinase C-responsive promoter”) is modulated by a GPCR signaling pathway. The cells of the invention include a G-protein that is capable of coupling to the GPCR. Genes encoding numerous GPCRs have been cloned (Simon et al., 1991, Science 252:802-808), and conventional molecular biology techniques can be used to express a GPCR on the surface of a cell of the invention. In one embodiment, the GPCR activated responsive promoter (e.g., the promoter operatively linked to the reporter polypeptide coding region) allows only a relatively short lag (e.g., less than 10, 15, 30, 60, 90, 120 or greater than 120 minutes) between engagement of the GPCR and transcriptional activation. In one embodiment, a responsive promoter (e.g., the promoter operatively linked to the reporter polypeptide coding region) includes the nuclear factor of activated T-cell promoter (Flanagan et al., 1991, Nature 352:803-807).

For example, the invention provides a method for determining whether a “target” polypeptide is a GPCR for a given ligand. One embodiment, involves expressing a target polypeptide in a cell described herein that comprises a reporter gene construct (e.g., a construct encoding a beta-lactamase reporter polypeptide operatively linked to a NFAT promoter). In this embodiment, the test polypeptide is contacted with a chosen ligand, usually of established activity, and a change in reporter polypeptide expression is detected. A “target” polypeptide, which is usually a GPCR, is any polypeptide expressed by a cell that can be assayed for activity using the present invention.

In some embodiments a GPCR ligand(s) includes, but is not limited to, light (e.g., photons), peptides, neurotransmitters, amino acids, hormones, lipids and chemokines.

Methods can be used to test ligands and compounds using GPCRs of known, partially known and unknown function. A test ligand is a molecule that can be assayed for its ability to bind to a GPCR. In some embodiments, a test compound is an antibody or a fragment thereof. Methods for obtaining fragments of antibodies capable of binding their respective antigen are known in the art. A test compound can be a molecule that can be assayed for its ability to modulate a signal transduction. Often, such a target polypeptide, test ligand, or test compound is, because of its sequence or structure, suspected of being able to function in a given capacity. Nonetheless, randomly chosen target polypeptides, test ligands, and test compounds also can be used in methods described herein, and with techniques known in the art or developed in the future. For example, expression of target polypeptides from nucleic acid libraries, can be used to identify proteins involved in signal transduction, such as orphan GPCRs. For instance, this technique can be used to identify physiologically responsive receptors (e.g., taste-responsive GPCRs) where the ligand responsible for inducing a physiological event is known (e.g., a given taste sensation is known).

The invention also includes enhancement of reporter polypeptide expression in a signal transduction detection system. This is particularly useful for improving the signal to noise ratio in a screening assay. It generally involves contacting the cell with a molecule (“subthreshold regulating molecule”) that alters the activity of a cellular process to a level subthreshold to the activation of a cellularly responsive control sequence that is operatively linked to the reporter polypeptide coding region. Because the level of cellular activity is subthreshold, the reporter coding region has a low expression level. The reporter gene system, however, is poised for activation by a change in cellular process induced by a test chemical, test ligand or expression of target protein. Such cellularly responsive control sequences can be responsive elements known in the art in other applications. Such response elements, however, do not need be responsive to their naturally occurring signal, since the assay may occur in cells lacking the required constituents for activation by a naturally occurring signal. The subthreshold regulating molecule can either increase or decrease the activity of the cellular process. It is understood that the cellular process may not only be “classic” cellular process, such as an enzymatic activity, but it also includes levels of cellular entities (e.g., ions, metabolites and second messengers) or other measurable properties of the cell (e.g., cell volume, chromatin density, etc.). Cells described herein can be used for this method. Other cells, however, can be used as well which express G-alpha-proteins endogenously, or heterologously.

For example, in order to enhance detection of expression of a reporter polypeptide, the cell can be contacted with a compound (e.g., a calcium ionophore) that increases calcium levels inside of the cell. By increasing calcium levels inside the cell, the probability that activation of a G-protein will activate expression of a reporter coding region can be greatly enhanced. In some embodiments, calcium levels are increased to a level that is just below the threshold level for activation of a calcium-responsive promoter, such as an NFAT promoter. In practice, ionomycin typically is added at a concentration of about 0.01 to about 3 μM or about 0.03 μM. Cells described herein can be used for this method. Other cells, however, can be used as well which express G-alpha-proteins endogenously, or heterologously.

In an alternate method of enhancing a signal transduction detection system, thapsigargin is added to the cell to set intracellular calcium levels at subthreshold levels to enhance reporter gene activation. Thapsigargin is added to the cell at a concentration of about 1 to about 50 nM, with the effect of partially depleting intracellular calcium pools and slowing the re-filling of such pools (Thastrup et al., 1990, Proc. Natl. Acad. Sci. 87:2466-2470). If desired, thapsigargin can be used at a higher concentration (e.g., about 200 nM to about 1 μM) in a “Ca₂-clamp” protocol, in which membrane potential is used to set the baseline calcium concentration (Negulescu et al., 1994, Proc. Natl. Acad. Sci. 91:2873-2877). This can be applied to screening for modulators of signal transduction using a reporter gene system with a calcium-responsive promoter. Cells described herein can be used for this method. Other cells, however, can be used as well which express G-alpha-proteins endogenously, or heterologously.

In yet another method of the invention, conventional molecular biology techniques can be used to express a calcium modulating ligand in cells, and thereby increase calcium levels (Bram et al., 1994, Nature 371:355-358). This can be applied to screening for modulators of signal transduction using a reporter gene system with a calcium-responsive promoter. Cells described herein can be used for this method. Other cells, however, can be used as well which express G-alpha-proteins endogenously, or heterologously.

In practicing these methods, it is preferable to add the ionophore to a level that is just below the threshold level for activation of the calcium-responsive promoter (e.g., the NFAT promoter). Expression of the reporter polypeptide then is activated by activation of the GPCR protein, and the subsequent rise in intracellular calcium levels

A related method of the invention for enhancing detection of expression of the reporter polypeptide involves contacting the cell with an activator of protein kinase C. Typically, this method involves contacting the cell with about 0.01 nM to about 3 mM or about 1 nM to about 3 nM of phorbol myristate acetate (PMA) or another phorbol ester. In one embodiment, PMA is used at a concentration of about 3 nM. The PMA concentration can be titrated to achieve sub threshold levels. Although PMA does not, by itself, affect NFAT-regulated gene expression, it potentiates a cell's response to an increase in calcium levels. Various analogs of PMA that retain this activity are known in the art, and can be used in the invention. Cells described herein can be used for this method. Other cells, however, can be used as well which express a GPCR endogenously or heterologously.

The invention also provides a method for determining whether a “test” ligand is a ligand for a given GPCR or other signaling pathway component. In this method, a selected GPCR or other signaling pathway component is expressed in a cell, such as a cell of the invention, e.g., that encodes a reporter polypeptide. The cell is contacted with a test ligand, and a change, if any, in expression of the reporter coding region is detected. This method is particularly well suited for identifying a ligand not known to bind to the receptor and it can also be used to determine receptor selectivity. In this method, the change in expression of the reporter coding region can be compared for a sample of cells in the presence, versus in the absence, of the test ligand in order to identify ligand specific activation. Cells described herein can be used for this method. Other cells, however, can be used as well which express G-alpha-proteins endogenously, or heterologously.

The aforementioned methods can readily be adapted to provide a method for characterizing the ability of a ligand to interact with a panel of GPCRs or other signaling pathway components of interest. Using GPCRs as examples, in such an assay, the first GPCR of interest is expressed in a cell, such as a cell of the invention that contains a construct encoding a reporter polypeptide. In a second cell (in a second, separate sample), a second GPCR of interest is expressed along with a reporter gene system. Additional GPCRs can be expressed in additional cells with reporter gene systems. Typically, these cells differ only with respect to the GPCR that is expressed. Each sample of cells is contacted with the “test” ligand of interest, and a change in reporter polypeptide expression is detected for each cell sample. By comparing the changes in expression of the reporter polypeptide between cell samples, one can characterize the functional activity of the ligand. This method is particularly well suited for assaying the ability of a known ligand to interact with several GPCRs that are known to be related. Thus the selectivity of the ligand can be determined. For example, various muscarinic receptors (e.g., M₁, M₂, and M₃) can be expressed, separately, on a cell. If desired, various modulators of G-protein activity (e.g., agonists and antagonists) can be characterized in a variation of this method. Cells described herein can be used for this method. Other cells, however, can be used as well which express G-alpha-proteins endogenously, or heterologously.

The invention also provides a general method for determining whether a test compound modulates signal transduction in a cell. This method also employs a cell, such as a cell of the invention, that includes a construct, that encodes a reporter polypeptide. In this method, the cell expresses a GPCR or other signaling pathway component, and the cell is contacted with a ligand that, in the absence of a test compound, activates signal transduction. The cell is also contacted with a test compound, and a change in expression of the reporter polypeptide indicates that the test compound modulates signal transduction in the cell.

In another embodiment, a compound being identified is a modulator of a GPCR (e.g., an agonist or an antagonist or an inverse agonist for the GPCR being assayed) or other signaling pathway component. In specific embodiments, the compound being identified is a natural and/or surrogate ligand for an orphan GPCR being assayed.

In lieu of contacting the cell with a ligand, the cell is contacted with a compound that directly activates a G-alpha-protein encoded by a construct within the cell. Examples of such compounds include mastoparan (Calbiochem) and aluminum fluoride. These compounds typically are used at concentrations of 0.5 to 5 mM. A change in expression of a reporter polypeptide indicates that the test compound modulates signal transduction in the cell. Such a change also indicates that the compound affects signaling events that occur subsequent to receptor signaling in the signaling pathway.

In another embodiment, a compound that directly activates a G-alpha-protein (e.g., mastoparan and aluminum fluoride) is used as a positive control. This compound can be a positive control for at least one of the pathways capable of being activated by the GPCR.

Some embodiments of the invention offer several advantages. By employing, in one embodiment, a particular GPCR, the invention allows the use of a single intracellular signaling pathway (e.g., activation of PLC-beta.) to analyze GPCRs. When a promiscuous G-protein is employed the invention allows analysis of GPCRs that may normally couple specifically to G-proteins of a single family. By providing methods that employ living cells, the invention allows a GPCR that is identified in an assay to be cloned. By employing fluorescent detection methods, the invention, in various embodiments, allows a practitioner to characterize and even isolate a single cell (e.g., with a desired characteristic). Accordingly, convenient cell-sorting methods, such as FACS, can be used to analyze and isolate cells. Fluorescent assays employed in the invention also provide a stable, non-labile indicator of G-protein activation. Such a stable signal (e.g., lasting 0.5, 1, 2, 3, 4, 5, 6, 7, 8, 9, 10, 11, 12 or more than 12 hours) allows a practitioner to analyze, for example, numerous samples in parallel, thus rendering the invention useful for high throughput screening of “test” polypeptides, ligands, and compounds. In one embodiment, the invention provides an assay for associating activation of a GPCR with gene expression, as detected by a fluorescence emission. In one embodiment, by providing methods for enhancing detection of G-protein activation, the invention provides a sensitive assay for detecting low levels, or brief activation, of a G-protein.

Methods of the invention may be carried out in numerous formats known in the art. In some embodiments of the inventions, the cells are analyzed/screened in 6-, 12-, 24, 96-, 384-, 1536-, and 3456-well plates. In some embodiments, the plates are black-wall, clear-bottom plates. In some embodiments, the culture plates are Poly-Lysine coated plates, e.g., BD BioCoat™ Poly-Lysine Cellware. In one embodiment, the cells are grown in serum-free media. In one embodiment, the cells are grown in serum that has reduced levels of tetracycline or other compounds that may modulate or interfere with modulation of the promoter for the reporter coding region.

In some embodiments, it may also be desirable to test the effects of serum starvation on the GPCR induced response as well as to control the pH of the buffer in order to avoid unexpected inhibition or activation of the receptor

Embodiments of the invention include various parameters with which related methods can be performed. The following are non-limiting examples of such parameters. Serum conditions: In some embodiments, the assay may be performed in the presence of 10%, 2%, 1%, or 0% serum. Cell density/well: In some embodiments, the assay may be performed at 2.5K, 5K, 10K, and 20K cells/well in a 384 well plate. DMSO tolerance: In some embodiments, the assay may be performed at 0%, 0.25%, 0.5%, and 1%. Stimulation kinetics: In some embodiments, the assay may be performed after a 3, 4, or 5 hour stimulation with a ligand (e.g., LPC) or test compound. Substrate loading time: In some embodiments, assay performance may be tested after 60 min, 90 min, and 120 min loading with the substrate. Assay reliability: In some embodiments, the Z′ for the assay is greater than 0.5, 0.6, 0.7, 0.8, 0.9 0.95, or 0.97. Optimization of each assay or assay conditions may be desired and such optimization is within the skill of in the art and can be performed without undue or burdensome experimentation.

A GPCR, G-protein and/or reporter coding regions, as well as essentially any other nucleic acid, may be introduced into the parental cell line through any transfection/infection method. For example, liposomal transduction with LIPOFECTAMINE™ 2000 (Invitrogen Corp., Carlsbad, Calif.) or through electroporation. Methods of transduction can be dependent upon the cellular background chosen. HEK and CHO cell lines are readily transfected through liposomal methods whereas Jurkat cells typically show poor transfectability with liposomal methods.

Following introduction of a nucleic acid, cells stably expressing all or part of this nucleic acid (e.g., the GPCR) can be selected if a selection marker was encoded on the nucleic acid (e.g., G418 selection).

Alternatively, viral transduction methods could be used to achieve greater transduction efficiency with difficult to transfect cell lines such as Jurkat cells.

Embodiments of the invention include a viral transduction system, such as the ViraPower Lentiviral Expression System from Invitrogen (Carlsbad, Calif.). Lentiviral transduction systems can result in greater transduction efficiency as both dividing and non-dividing cells can be efficiently transduced by this method.

Vectors of the invention may include a coding region for a selectable marker. Selection markers include, but are not limited to, neomycin resistance, hygromycin resistance, puromycin resistance, Blasticidin and the Blasticidin Selection Marker, and a Zeocin™ Selection Marker.

One embodiment of the invention provides a method of constructing a GPCR reporter cell comprising: (a) introducing into the cell a nucleic acid comprising a promoter operatively linked to a GPCR coding region and (b) introducing into the cell a nucleic acid comprising a second promoter operatively linked to a second coding region for a reporter polypeptide. Embodiments of the invention include, wherein (a) is performed prior to (b); (b) is performed prior to (a); or (a) and (b) are performed essentially simultaneously. Other embodiments, including various combinations, include wherein the second promoter is regulated directly or indirectly by the amount of activated GPCR; wherein the second promoter regulates expression by the amount of or change in intracellular calcium amounts; wherein the second promoter comprises a responsive element selected from the group consisting of an NFAT responsive element, a cAMP responsive element (CRE) and kinase C-responsive promoter; wherein the NFAT responsive element comprises the nucleotide sequence of SEQ ID NO:1; wherein the second promoter is regulated by the amount of or change in cAMP amounts; wherein the cAMP responsive element comprises the nucleotide sequence of SEQ ID NO:2; wherein the reporter polypeptide is detected directly or indirectly by fluorescence, light absorption, calorimetric readout, detecting an enzyme reaction, immunohistochemistry, immunofluorescence, flow cytometry, fluorescent-activated cell sorting (FACS), luminescence or FRET; wherein the reporter polypeptide is selected from the group consisting of a beta-lactamase, a fluorescent polypeptide, a luciferase, a green fluorescent protein (GFP), a chloramphenicol acetyl transferase, an alkaline phosphatase a beta.-galactosidase, an alkaline phosphatase, and a human growth hormone; wherein expression of the reporter polypeptide is increased when the amount of activated GPCR is increased; wherein the expression of the reporter polypeptide is decreased when the amount of activated GPCR is increased; wherein expression of the reporter polypeptide is increased when the amount of activated GPCR is decreased; wherein expression of the reporter polypeptide is decreased when the amount of activated GPCR is decreased; wherein the cell is stable; and any possible combination of the individual embodiments.

Some aspects of the invention, provide a method of detecting or monitoring activity of a GPCR comprising: (a) culturing a cell described herein under conditions wherein the GPCR is expressed; and (b) detecting the expression of the reporter polypeptide.

One embodiment of the invention provides a method for measuring the ability of a compound to affect or modulate activation of a GPCR comprising: (a) culturing a cell described herein under conditions wherein the GPCR is expressed; (b) contacting the cell with the compound; and (c) measuring expression of the reporter polypeptide.

In one embodiment, the measuring expression is performed in the presence and absence of the compound. In one embodiment, the compound is determined to modulate activation of a GPCR if the measured expressions in the presence and absence of the compound differ. In one embodiment, the measured expressions in the presence and absence of the second compound have a statistically significant difference.

The invention provides in one embodiment, a method for determining whether binding of a first compound to a GPCR is capable of being modulated by a second compound comprising: (a) culturing a cell described herein under conditions wherein the GPCR is expressed and contacting the cell with the first compound to form a first sample; (b) culturing a cell described herein under conditions wherein the GPCR is expressed and contacting the cell with the first compound and second compound to form a second sample; and (c) measuring expression of the reporter polypeptide in the first and second samples.

In one embodiment, expression is measured in the presence and absence of the second compound. In one aspect of the invention, the second compound is determined to modulate binding of a GPCR if the measured expressions in the presence and absence of the second compound differ. In one embodiment, the second compound is determined to modulate binding of a GPCR if the measured expressions in the presence and absence of the second compound are statistically significantly different.

In some aspects of the invention, the culturing of a cell is in the presence of a factor that induces expression of the GPCR. In one embodiment, the inducing agent (e.g., for the inducible or regulatable promoter) is tetracycline, doxycycline or a heavy-metal. In one embodiment, the promoter of the GPCR is heat inducible.

Some embodiments of the invention comprise contacting the cell with a calcium increasing compound that increases calcium levels inside the cell. In one embodiment, the calcium increasing compound is ionomycin or thapsigargin. In one embodiment, a method of the invention further comprises contacting the cell with phorbol myristate acetate or an analog thereof.

Some embodiments of the invention provide methods of identifying a GPCR for a ligand or of identifying a ligand for a GPCR, the method comprising: (a) expressing the GPCR in a cell described herein; (b) contacting the cell with the ligand; and (c) detecting expression of the reporter polypeptide. Some embodiments comprise contacting the cell with a reporter polypeptide substrate.

### Utilizing Gene Expression Knockdown Methods in the Present Invention

The present invention also provides methods of determining an appropriate cellular background and/or pathway which shows sufficient agonist induced activation of the beta-lactamase reporter over the basal levels of beta-lactamase expression which may be present in the cell.

The present invention also provides methods of verifying that detection of activation is actually caused by activation of the GPCR or other signaling pathway component. For example, cells that display characteristics of the GPCR being activated can be treated with a compound that “knocks down” (i.e., decreases expression) expression of the GPCR. If the detection levels (e.g., fluorescence from a reporter polypeptide) decreases when expression of the GPCR is decreased (knocked-down), this suggests the reporter signal seen is a result of the activation state of the GPCR. Or if the expression of the GPCR is decreased, but the detection levels are unchanged or do not change accordingly, this suggests the activation characteristics (e.g., expression of the reporter polypeptide) are not due to activation of the GPCR. In one embodiment, the expression of the GPCR or other signaling pathway component is decreased using RNAi or anti-sense RNA specific for the GPCR or other signaling pathway component. In another embodiment, expression of the GPCR or other signaling pathway component is decreased by changing the concentration of a compound that regulates the promoter controlling expression of the GPCR or other signaling pathway component.

### Internal Ribosome Entry Sites

IRESs are used to express two or more proteins from a single vector. In some cases, these proteins are translated from a single mRNA transcript. An IRES sequence is commonly used to drive expression of a second, third, fourth coding sequence, etc.

IRES elements were first discovered in picornavirus mRNAs (Jackson et al. (1990) Trends Biochem Sci 15(12):477-S3; Jackson et al. (1995) RNA 1(10):985-1000). Examples of IRESs that can be used in accordance with the present invention include, but are not limited to, those from or derived from Picornavirus e.g., HAV (Glass et al. 1993. Virol 193:842-852), encephelomycarditis virus (EMCV) which is e.g., commercially available from Novagen (Duke et al. (1992) J. Virol 66(3):1602-9; Jang & Wimmer, 1990 Gene Dev 4:1560-1572), and Poliovirus (Borman et al., 1994. EMBO J 13:3149-3157); HCV (Tsukiyama-Kohara et al., 1992. J Virol 66:1476-1483) BVDV (Frolov I et al., 1998. RNA. 4:1418-1435); Leishmania virus, e.g., LRV-1 (Maga et al., 1995. Mol Cell Biol 15:4884-4889); Retroviruses e.g., MoMLV (Torrent et al., 1996. Hum Gene Ther 7:603-612), VL30 (Harvey murine sarcoma virus), REV (Lopez-Lastra et al., 1997. Hum Gene Ther 8:1855-1865); and Eukaryotic mRNA e.g. immunoglobulin heavy-chain binding protein (BiP) (Macejak & Sarnow, 1991. Nature 353:90-94), antennapedia mRNA (Oh et al., 1992. Gene & Dev 6:1643-1653), fibroblast growth factor 2 (FGF-2) (Vagner et al., 1995. Mol Cell Biol 15:35-44), PDGF-B (Bernstein et al., 1997. J Biol Chem 272:9356-9362), IGFII (Teerink et al., 1995. Biochim Biophys Acta 1264:403-408), translational initiation factor elF4G (Gan & Rhoads, 1996. J Biol Chem 271:623-626), insulin-like growth factor (IGFU), yeast transcription factors TFIID and HAP4, and the vascular endothelial growth factor (VEGF) (Stein et al., 1998. Mol Cell Biol 18:3112-3119; Huez et al., 1998. Mol Cell Biol 18:6178-6190) as well as those described in U.S. Pat. No. 6,692,736. IRESs have also been reported in different viruses such as cardiovirus, rhinovirus, aphthovirus, HCV, Friend murine leukemia virus (FrMLV) and Moloney murine I leukemia virus (MoMLV). As used herein, the term “IRES” encompasses functional variations of IRES sequences as long as the variation is able to promote direct internal ribosome entry to the initiation codon of a downstream cistron, leading to cap-independent translation. An IRES utilized in the present invention may be mammalian, viral or protozoan.

Thus, the product of a downstream cistron can be expressed from a bicistronic (or multicistronic) mRNA, without requiring either cleavage of a polyprotein or generation of a monocistronic mRNA. Commonly used internal ribosome entry sites are approximately 450 nucleotides in length and are characterized by moderate conservation of primary sequence and strong conservation of secondary structure. The most significant primary sequence feature of the IRES is a pyrimidine-rich site, whose start is located approximately 25 nucleotides upstream of the 3′ end of the IRES. (See Jackson et al. (1990) Trends Biochem Sci 15(12):477-S3.)

Three major classes of picornavirus IRES have been identified and characterized: (1) the cardio- and aphthovirus class (for example, the encephelomycarditis virus, Jang et al. (1990) Gene Dev 4:1560-1572); (2) the entero- and rhinovirus class (for example, polioviruses, Borman et al. (1994) EMBO J. 13:314903157); and (3) the hepatitis A virus (HAY) class, Glass et al. (1993) Viroll93:842-852). For the first two classes, two general principles apply. First, most of the about 450-nucleotide sequence of the IRES functions to maintain particular secondary and tertiary structures conducive to ribosome binding and translational initiation. Second, the ribosome entry site is an AUG triplet located at the 3′ end of the IRES, approximately 25 nucleotides downstream of a conserved oligopyrimidine tract. Translation initiation can occur either at the ribosome entry site (cardioviruses) or at the next downstream AUG (entero/rhinovirus class). Initiation occurs at both sites in aphthoviruses.

HCV and pestiviruses such as bovine viral diarrhea virus (BVDV) or; classical swine fever virus (CSFV) have 341 nt and 370 nt long 5′-UTR respectively. These 5′-UTR fragments form similar RNA secondary structures and can have moderately efficient IRES function (Tsukiyama-Kohara et al. (1992) J. Virol. 66:1476-1483; Frolov I et al., (1998) RNA 4:1418-1435). Recent studies showed that both Friend-murine leukemia virus (MLV) 5′-UTR and rat retrotransposon virus-like 30S (VL30) sequences contain IRES structure of retroviral origin (Torrent et al. (1996) Hum Gene Ther 7:603-612).

In eukaryotic cells, translation is normally initiated by the ribosome scanning from the capped mRNA 5′ end, under the control of initiation factors. However, several cellular mRNAs have been found to have IRES structure to mediate the cap-independent translation (van der Velde, et al. (1999) Int J Biochem Cell Biol. 31:87-106). Examples are immunoglobulin heavy-chain binding protein (BiP) (Macejak et al. (1991) Nature 353:90-94), antennapedia mRNA of Drosophilan (Oh et al. (1992) Gene and Dev 6:1643-1653), fibroblast growth factor-2 (FGF-2) (Vagner et al. (1995) Mol Cell Biol 15:35-44), platelet-derived growth factor B (PDGF-B) (Bernstein et al. (1997) J Biol Chem 272:9356-9362), insulin-like growth factor II (Teerink et al. (1995) Biochim Biophys Acta 1264:403-408), the translation initiation factor eIF4G (Gan et al. (1996) J Biol Chem 271:623-626) and vascular endothelial growth factor (VEGF) (Stein et al. (1998) Mol Cell Biol 18:3112-3119; Huez et al. (1998) Mol Cell Biol 18:6178-6190).

An IRES may be prepared using standard recombinant and synthetic methods known in the art. For cloning convenience, restriction sites may be engineered into the ends of the IRES fragments to be used.

### Self-Processing Cleavage Sites or Sequences

Although the mechanism is not part of the invention, the activity of self-processing cleavage site, self-processing cleavage sequence or a 2A-like sequence are used interchangeably and may involve ribosomal skipping between codons which prevents formation of peptide bonds (de Felipe et al., Human Gene Therapy 11: 1921-1931 (2000); Donnelly et al., J. Gen. Virol. 82:1013-1025 (2001)), although it has been considered that the domain acts more like an autolytic enzyme (Ryan et al., Virol. 173.35-45 (1989).

A “self-processing cleavage site” or “self-processing cleavage sequence” refers to a DNA or amino acid sequence, wherein upon translation, rapid intramolecular (cis) cleavage of a polypeptide comprising the self-processing cleavage site occurs to result in expression of discrete mature protein or polypeptide products. Also, a “self-processing cleavage site” or “self-processing cleavage sequence” refers to a DNA or amino acid sequence, wherein upon translation, the sequence results in “ribosomal skip” as known in the art and described herein. A “self-processing cleavage site”, may also be referred to as a post-translational or co-translational processing cleavage site, exemplified herein by a 2A site, sequence or domain. It has been reported that a 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly et al. 2001 J Gen Virol. 82:1013-25). Alternatively, a “self-processing cleavage site”, “self-processing cleavage sequence” or a 2A sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

Although the mechanism is not part of the invention, the activity of a 2A-like sequence or self-processing cleavage site may involve ribosomal skipping between codons which prevents formation of peptide bonds (de Felipe et al., Human Gene Therapy 11: 1921-1931 (2000); Donnelly et al., J. Gen. Virol. 82:1013-1025 (2001)), although it has also been considered that the domain acts more like an autolytic enzyme (Ryan et al., Virol. 173.35-45 (1989).

The Foot and Mouth Disease Virus 2A oligopeptide has previously been demonstrated to mediate the translation of two sequential proteins through a ribosomal skip mechanism (Donnelly et al. 2001 J Gen Virol. 82:1013-25; Szymczak et al., Nat. Biotechnol. 2004 (5):589-94; Klump et al., Gene Ther. 2001 (10):811-7; De Felipe et al., Hum Gene Ther. 2000 11(13):1921-31; Halpin et al., Plant J. 1999 17(4):453-9; Mattion et al., J. Virol. 1996 70(11):8124-7; and de Felipe P. et al. Gene Ther. 1999 6(2):198-208). Multiple proteins are encoded as a single open reading frame (ORF). During translation in a bicistronic system, the presence of the FMDV 2A sequence at the 3′ end of the upstream gene abrogates the peptide bond formation with the downstream cistron, resulting in a “ribosomal skip” and the attachment of the translated FMDV 2A oligopeptide to the upstream protein (Donnelly et al., J Gen Virol. 2001 82(Pt 5): 1013-25). Processing occurs in a stoichiometric fashion, estimated to be as high as 90-99%, resulting in a near molar equivalency of both protein species (Donnelly et al., J Gen Virol. 2001 82(Pt 5):1027-41). Furthermore, through deletion analysis the amino acid sequence-dependent processing activity has been localized to a small section at the c-terminal end of the FMDV 2A oligopeptide (Ryan et al., EMBO J. 1994 13(4):928-33). Most members of the Picomavirus family (of which FMDV belongs) use similar mechanisms of cotranslational processing to generate individual proteins (Donnelly et al., J Gen Virol. 2001 82(Pt 5):1027-41). In fact, publications have shown that fragments as small as 13 amino acids can cause the ribosomal skip (Ryan et al., EMBO J. 1994 Feb. 15; 1 3(4):928-33). Incorporation of truncated versions of the peptide in bicistronic vector systems has demonstrated that almost all of the processing activity is preserved even in non-viral vector systems (Donnelly et al. J Gen Virol. 2001 82(Pt 5):1027-41). At least four coding sequences that have been efficiently expressed under a single promoter by strategic placement of these types of elements (Szymczak et al. Nat. Biotechnol. 2004 22(5):589-94.). Therefore, self-processing cleavage sites such as the FMDV 2A oligopeptide may be utilized in the present invention to link expression of the heavy and light chain coding regions.

For the present invention, the DNA sequence encoding a self-processing cleavage site is exemplified by viral sequences derived from a picornavirus, including but not limited to an entero-, rhino-, cardio-, aphtho- or Foot-and-Mouth Disease Virus (FMDV). In one embodiment, the self-processing cleavage site coding sequence is derived from a FMDV.

The FMDV 2A domain is typically reported to be about nineteen amino acids in length (e.g., LLNFDLLKLAGDVESNPGP (SEQ ID NO: 3); TLNFDLLKLAGDVESNPGP (SEQ ID NO: 4), Ryan et al., J. Gen. Virol. 72.2727-2732 (1991)), however oligopeptides of as few as thirteen amino acid residues (e.g., LKLAGDVESNPGP (SEQ ID NO: 5)) have also been shown to mediate cleavage at the 2A C-terminus in a fashion similar to its role in the native FMDV polyprotein processing. Alternatively, a vector according to the invention may encode amino acid residues for other 2A-like regions as discussed in Donnelly et al., J. Gen. Virol. 82:1027-1041 (2001) and including but not limited to a 2A-like domain from picornavirus, insect virus, Type C rotavirus, trypanosome repeated sequences or the bacterium, Thermatoga maritime.

Variations of the 2A sequence have been studied for their ability to mediate efficient processing of polyproteins (Donnelly et al., J. Gen. Virol. 82:1027-1041 (2001)). Such variants are specifically contemplated and encompassed by the present invention. In one embodiment, the 2A sequence is a variant 2A sequence.

Further examples and descriptions of self-processing cleavage sites and vectors encoding them are found in U.S. Patent Publication Nos. 2005/0042721 and 2005/0003482, the entire disclosures of which are incorporated herein by reference.

### Kits and Product Literature of the Invention

If desired (e.g., for commercial purposes), a cell(s), a construct, a written method and/or a reagent of the invention can be packaged into a container that is packaged within a kit. Such a kit may also contain any of the various isolated nucleic acids, antibodies, proteins, signal transduction detection systems, substrates, and/or compounds described herein, known in the art or developed in the future. In one embodiment, a kit also includes a set of instructions for any or all methods described herein.

The kits can be produced to accomplish methods described herein. Such kits can include the polynucleotides for GPCR expression, cells for GPCR expression or G-alpha-protein expression and signal transduction detection systems, such reporter gene systems.

A kit comprising assay reagents and a container containing a cell of the invention. A kit comprising a nucleic acid of the invention. In one embodiment, comprises a protocol for methods of the invention.

The invention also includes product literature (e.g., catalogs, brochures, instructions, etc.). One example of product literature of the invention is a protocol for using kits of the invention. This protocol may be in electronic or tangible (e.g., printed on paper) forms. Thus, is some embodiments, the invention includes a composition of matter comprising a sheet of paper with method of the invention printed on it. Exemplary protocols of the invention are set out in the examples below.

All publications, patents and patent applications mentioned in this specification are herein incorporated by reference into the specification to the same extent as if each individual publication, patent or patent application was specifically and individually indicated to be incorporated herein by reference.

### 8. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

Whereas, particular embodiments of the invention have been described herein for purposes of description, it will be appreciated by those skilled in the art that numerous variations of the details may be made without departing from the invention as described in the appended claims.

### Example 1

### Vector Construction

During the course of experimentation, it was discovered that G2A exhibited constitutive activity when expressed from a CMV promoter in Freestyle™293F cells. However, this constitutive activity led to cell death. Therefore, work was performed to control the level of G2A expression in order to prevent this unwanted cell death.

To achieve this, the T-REx™ expression system from Invitrogen was utilized as an exemplary embodiment. The T-REx™ system uses a repressor mechanism that blocks transcription from the powerful CMV promoter in the absence of tetracycline.

Components of the T-REx™ are illustrated in FIG. 2. Two tetracycline operator sequences (TetO2) were inserted between the TATA box of the CMV promoter and the transcriptional start site. The TetO2 sequence itself has no effect on expression. Although applicants do not wish to be bound by any theoretical speculation as to the mechanistic explanation of the invention described, when the tetracycline repressor protein (TR) is present, it effectively binds the TetO2 sites and blocks transcription initiation. Tetracycline added to the culture medium binds to, and changes the conformation of, the TR protein. This change causes the TR protein to release the TetO2 sites, derepressing transcription from the CMV promoter. The result is high-level expression of the gene of interest. Expression levels can be modulated based on the tetracycline concentration and can be induced to levels that are achieved with constitutive CMV expression vectors.

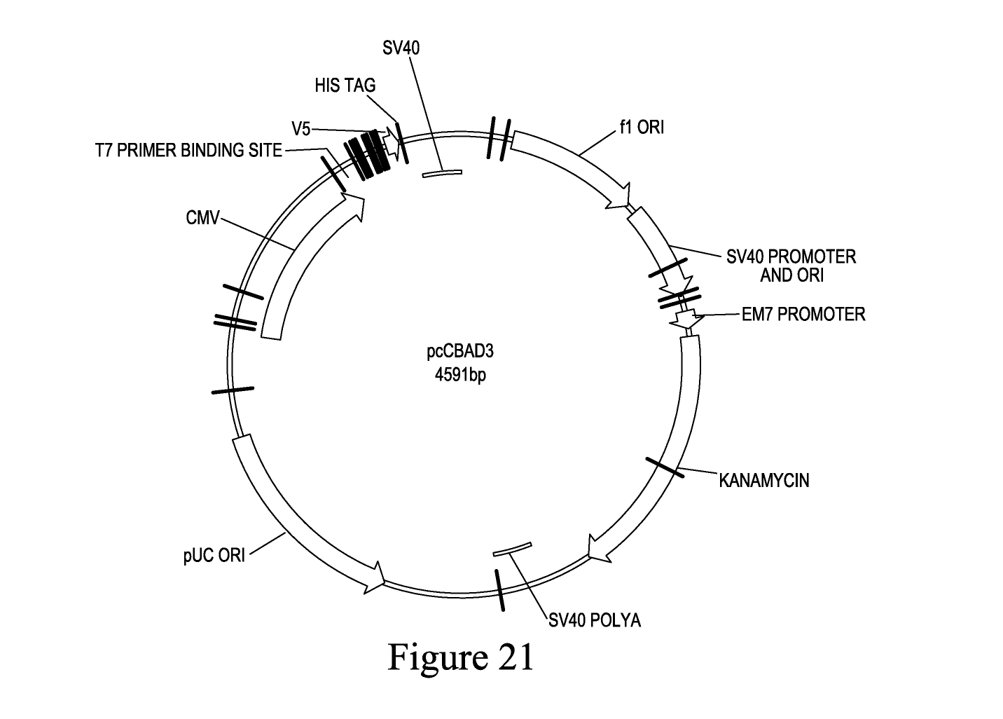

To create the T-REx™-G2A-NFAT-bla Freestyle™293F cell line, G2A was cloned from a cDNA library prior to sub-cloning into a pcDNA5/TO expression plasmid as follows. pcDNA5 G2A/TO was created by starting with pcDNA5/TO (Invitrogen Catalog #V1033-20) and was G2A cloned from a pool of cDNA libraries (heart, liver, lung, spleen, brain) (Clontech, Mountain View, Calif.) with the following primers: G2arevbamHI-TATCATGGATCCTCAGCAGGACTCCTCAATCAG (SEQ ID NO:9) and G2aforNHE CAAGCTGGCTAGCCACCATGTGCCCAATGCTACTG (SEQ ID NO:10). The PCR product was cloned into pcCBAD3 (SEQ ID NO:11, FIG. 21) using the NHE and BAMH1 sites. Next it was cut out of pcCBAD3 with NHE and XMA. pcDNA5/TO was cut with the restriction enzymes XMA and XBA. G2A was inserted into pcDNA5/TO using the XMA and XBA(NHE) sites to create pcDNA5 G2A/TO. XBA and NHE have compatible sites. This expression plasmid also contains a hygromycin antibiotic resistance gene. The sequence of the G2A insert was verified to match GenBank Accession #NM_{—}013345. A map of the resulting expression plasmid pcDNA5 G2A/TO is shown in FIG. 3.

The pcDNA6/TR plasmid was also used during construction of the T-REx™-G2A-NFAT-bla Freestyle™293F cell line to provide expression of the TR. A plasmid map is shown in FIG. 4.

### Example 2

### Transfection/Selection

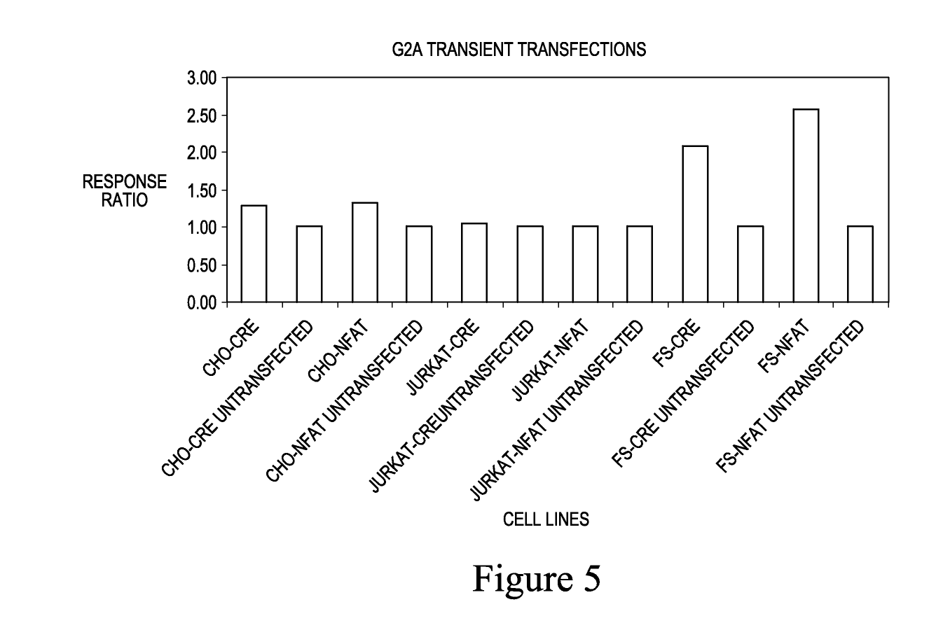

Transient transfections—Several parental cell lines were transiently transfected with G2A under a non-regulated CMV promoter to assess feasibility for stable cell line generation. Invitrogen's (Carlsbad, Calif.) NFAT-bla CHO-K1, (Catalog #K1078) NFAT-bla Freestyle™ HEK 293F (Catalog #K1097), NFAT-bla Jurkat (Catalog #K1077), CRE-bla CHO-K1 (Catalog #K1129), CRE-bla Freestyle™ HEK 293F (Catalog #K1130), and CRE-bla Jurkat parental CellSensor™ (Catalog #K1134) cell lines were transiently transfected with G2A. The transient transfections were carried out in 96 well plates using Invitrogen's Lipofectamine™ 2000. The results from the transient transfection are shown in FIG. 5.

Both NFAT-bla and CRE-bla Freestyle™ HEK 293F cell lines exhibited constitutive beta-lactamase expression upon G2A transfection. Stable cell lines were attempted in both Freestyle™293F cell lines, however the constitutive activity of G2A led to cell death in both cell lines which precluded stable assay development.

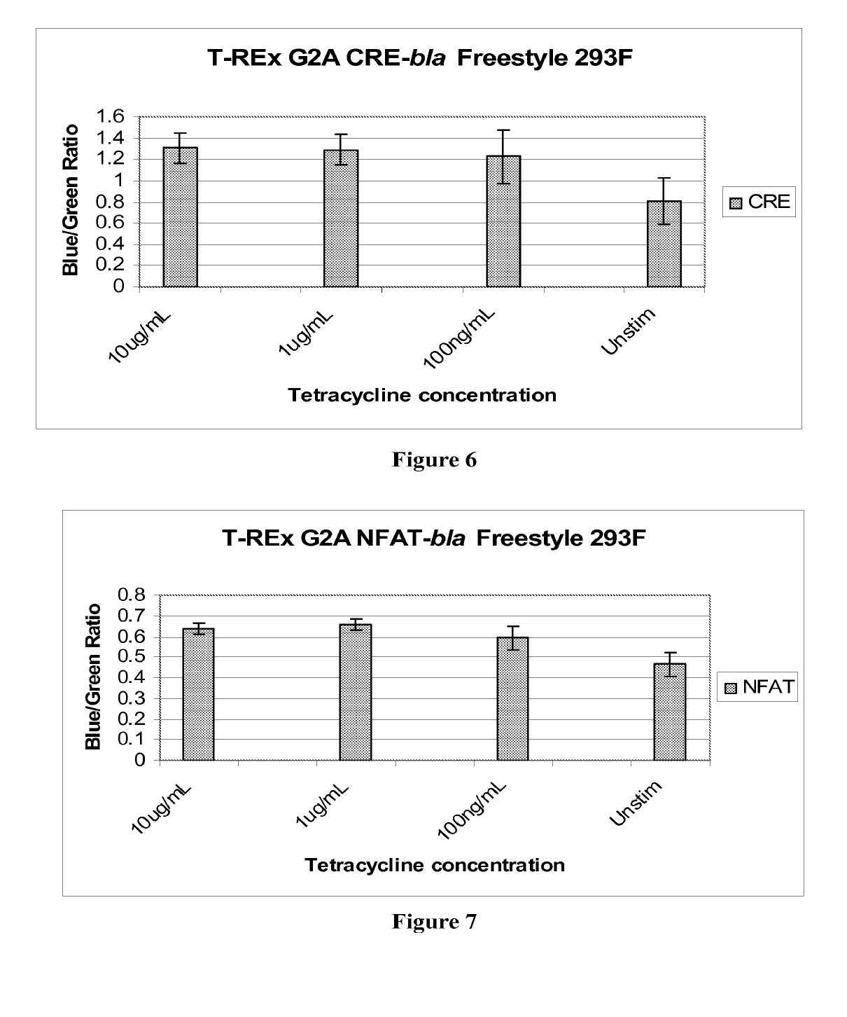

In order to control the levels of G2A expression, G2A was placed under the control of an inducible promoter using Invitrogen's T-REx™ system. To again assess feasibility, pcDNA 5 G2A/TO was transiently transfected into CRE-bla Freestyle™ and NFAT-bla Freestyle™ cell lines stably expressing the T-REx™ Tet repressor plasmid (pcDNA6/TR; e.g., catalog# V1025-20, Invitrogen, Carlsbad, Calif.). The transfected cells were then treated with 10 ug/mL, 1 ug/mL, or 100 ng/mL tetracycline for 24 h to induce G2A expression. The data is shown in FIGS. 6 and 7. Both T-REx™-G2A-NFAT-bla Freestyle™293F and T-REx™-G2A-CRE-bla Freestyle™293F lines had a response to tetracycline induction above the unstimulated transfected cells. Stable pools of cells were created in both the TR CRE-bla and TR NFAT-bla Freestyle™293F backgrounds.

Stable Transfections—Invitrogen's NFAT-bla and CRE-bla Freestyle™ HEK 293F CellSensor™ Cell Lines (Invitrogen, catalog# K1097 and K1130) were transfected with the T-REx™ plasmid pcDNA6/TR and selected with blasticidin to produce a stable Tet repressor (TR) cell lines. The TR lines were then transfected with T-REx™ plasmid pcDNA 5/TO G2A and selected with hygromycin for 2 weeks prior to sorting by flow cytometry.

### Example 3

### Sorting

Cells were trypsinized and loaded with Invitrogen's LiveBLAzer™-FRET B/G substrate for 2 hours prior to sorting. The stable T-REx™ G2A NFAT-bla Freestyle™ 293F and T-REx™ G2A CRE-bla Freestyle™ 293F pools were sorted without tetracycline stimulation into green and turquoise(P1) pools. Clones were obtained from the T-REx™ G2A NFAT-bla Freestyle™ 293F and T-REx™ G2A CRE-bla Freestyle™ 293F turquoise pools by a second sort to distribute single-cells from the turquoise population (P1) into each well of e.g., three 96-well plates.

### Example 4

### Clone Selection

Stimulation Kinetics—Selected pools of both CRE-bla and NFAT-bla T-REx™ G2A Freestyle™ cells were stimulated with tetracycline for 3, 4, 5, or 24 h and loaded for 2 h with 1 μM LiveBLAzer™-FRET B/G substrate.

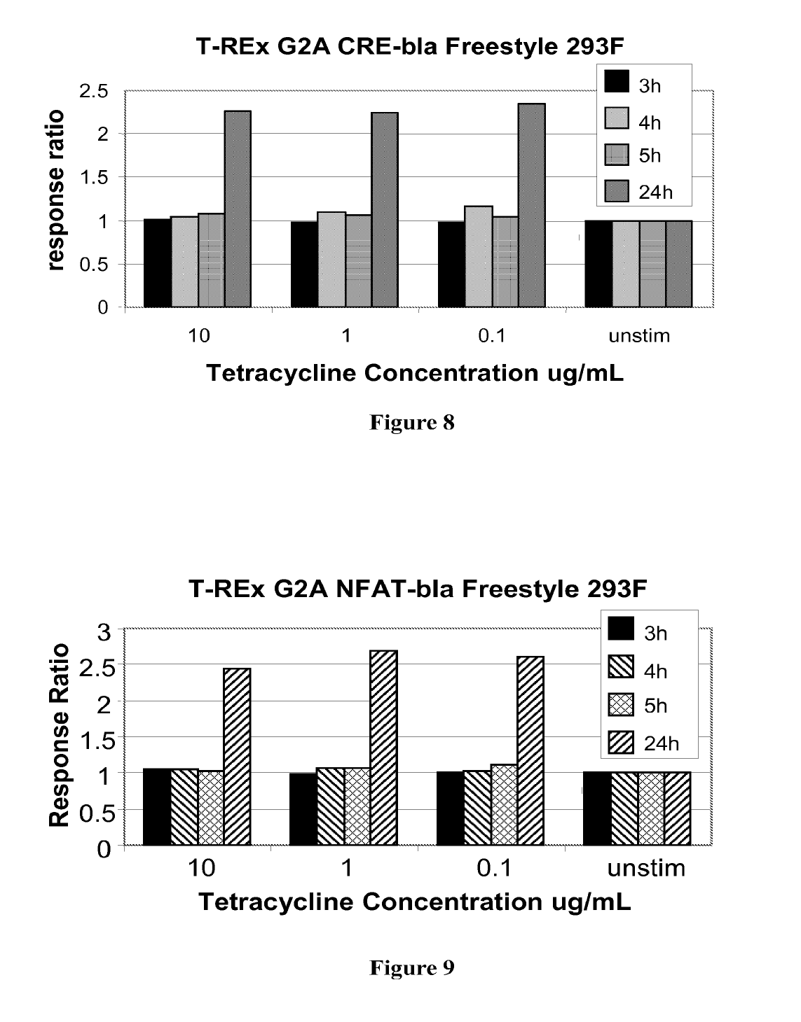

Data is shown in FIGS. 8 and 9. No beta-lactamase activity was seen after 3, 4, or 5 hours of tetracycline stimulation. 24 h did produce a beta-lactamase signal but substantial cell death was also seen.

### Example 5

### Modulation Using Tetracycline Concentrations

To improve the balance between tetracycline-induced signaling and cell death by testing shorter induction times, lower tetracycline concentrations and altered culture conditions were utilized to promote cell health. A 16 h tetracycline stimulation time was attempted on the pools from the first sort. Cells were plated at 10,000 cells/well 24 hours prior to assay in DMEM+10% dFBS and incubated at 37° C./5% CO2 until time of assay. The cells were stimulated for 16 hours (e.g., overnight) with a dilution series of the tetracycline. Following stimulation, the cells were loaded for 2 hours at room temperature in the dark with LiveBLAzer™-FRET B/G loading solution containing 1 μM substrate. This assay was done in Poly-D-Lysine coated plates.

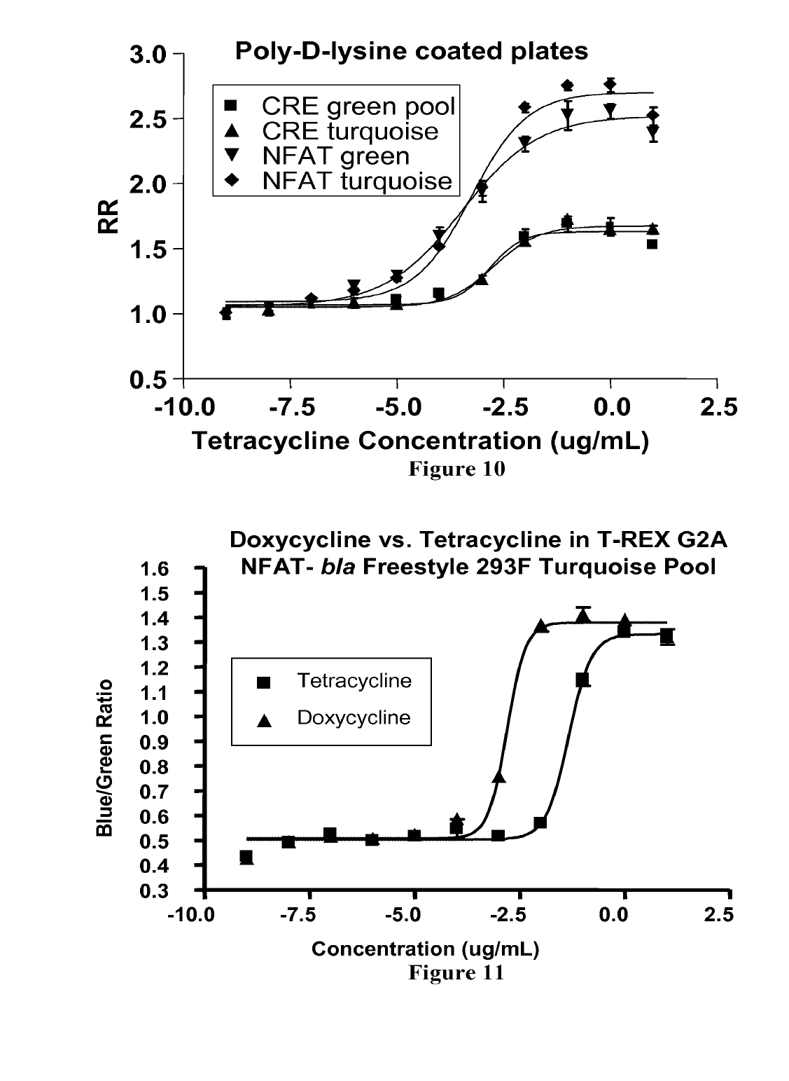

Related data can be seen in FIG. 10. This and the shorter induction time may have aided cell adhesion and survivability since much less cell death was observed. A 16 h stimulation and Poly-D-Lysine coated plates were used for all validation experiments.

### Example 6

### Clone Screening

Three 96-well plates of CRE-bla and NFAT-bla T-REx™ G2A Freestyle™ 293F cell clones were screened for optimal G2A responses. Each clone is split 1:3. An unstimulated well and a stimulated well were plated. A third well was plated into a separate plate for clonal expansion. T-REx™ G2A NFAT-bla Freestyle™ 293F and T-REx™ G2A CRE-bla Freestyle™ 293F clones chosen for further testing are shown in Tables 1 and 2, respectively.

TABLE 1

| | Stim B:G | Unstim B:G | Response |
|---|---|---|---|
| Clone # | Ratio | Ratio | Ratio |
| 4 | 2.17 | 0.34 | 6.30 |
| 11 | 3.20 | 0.80 | 3.99 |
| 20 | 2.03 | 0.38 | 5.30 |
| 22 | 2.08 | 0.35 | 5.88 |
| 32 | 3.14 | 0.66 | 4.80 |
| 38 | 2.46 | 0.41 | 6.01 |
| 40 | 2.94 | 0.44 | 6.66 |
| 46 | 3.97 | 0.74 | 5.40 |
| 47 | 7.18 | 2.26 | 3.18 |

TABLE 2

| | Stim B:G | Unstim B:G | Response |
|---|---|---|---|
| Clone # | Ratio | Ratio | Ratio |
| 4 | 1.74 | 0.42 | 4.14 |
| 18 | 1.51 | 0.42 | 3.70 |
| 24 | 7.24 | 1.50 | 4.82 |
| 27 | 4.05 | 0.98 | 4.12 |

The response ratios of the CRE-bla cell lines were generally lower than that of the NFAT-bla cell lines. The background was also higher in the CRE-bla clones. In consideration of these lower responses, T-REx™ G2A NFAT-bla Freestyle™ 293F clones #20, 40 and 46 were chosen for further study. These clones had shown good response ratios starting from a range of different background levels of constitutive beta-lactamase activity.

### Example 7

### Induction Optimization

Since G2A has no known agonists, doxycycline and tetracycline were compared as inducers of the assay. An antibiotic selected pool of cells were plated at 10,000 cells/well 24 hours prior to assay in DMEM+10% dFBS and incubated at 37° C./5% CO2 until time of assay. The cells were stimulated with a dilution series of each compound for 16 hours at 37° C./5% CO2 and loaded for 2 hours at room temperature in the dark with LiveBLAzer™-FRET B/G loading solution containing 1 μM substrate.

Surprisingly, both were shown to be inducers of the assay. Both produced similar response ratios, but doxycycline was ˜30 times more potent than tetracycline. Results indicating that either tetracycline or doxycycline can be used as inducers are shown in FIG. 11 and Table 3.

TABLE 3

| | | RR | Z′ |
|---|---|---|---|
| Agonist | EC₅₀ | (Max) | (Max) |
| Tetracycline | 46 ng/mL | 3.1 | 0.71 |
| Doxycycline | 1.6 ng/mL | 3.3 | 0.70 |

### Example 8

### Construction of the T-REx™-G2A-NFAT-bla Freestyle™293F Cell Line

The T-REx™-G2A-NFAT-bla Freestyle™293F cell line was created by co-transfecting expression plasmids containing the T-REx™ Tetracycline repressor protein (TR) and G2A under control of the T-REx™ Tet operator sequences (TetO2) and a CMV promoter into Invitrogen's NFAT-bla Freestyle™293F CellSensor™ cell line which is a part of Invitrogen's GeneBLAzer® technology portfolio.

### Example 9

### RT-PCR/RNAi Verification

In order to confirm that G2A induction was causing the constitutive beta-lactamase activity seen in these clones, RT-PCR and RNAi experiments were carried out. RNA was harvested from both doxycycline stimulated (16 hours) and unstimulated cells.

G2A expression was observed in all three clones both before and after stimulation, although expression increased upon doxycycline treatment.

Invitrogen's Stealth™ siRNA (e.g., Invitrogen, catalog# 1299003) was used to knockdown G2A expression to confirm that the observed increase in beta-lactamase blue:green ratios depends upon G2A expression.

For the experiment, cells were plated at 5,000 cells/well one day prior to transfection with siRNA (sequence 5′ to 3′: UAAGCCCAUGCUCUGCUUGAUGCUC (SEQ ID NO:6). 50 nM final siRNA concentration was used for transfections. Cells were treated with siRNA for 48 h followed by a 16 h doxycycline induction. An siRNA with medium GC content (Med GC) was used as a negative control, while siRNA directed towards beta-lactamase (BLA) was used as a positive control expected to knock down the beta-lactamase signal.

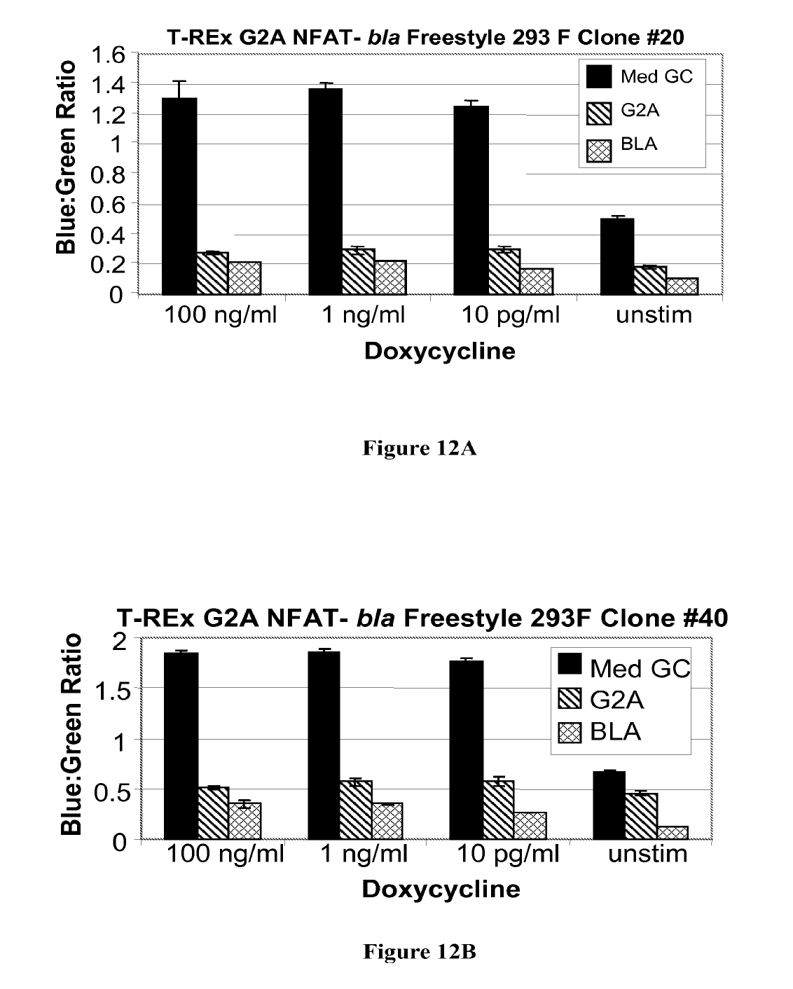

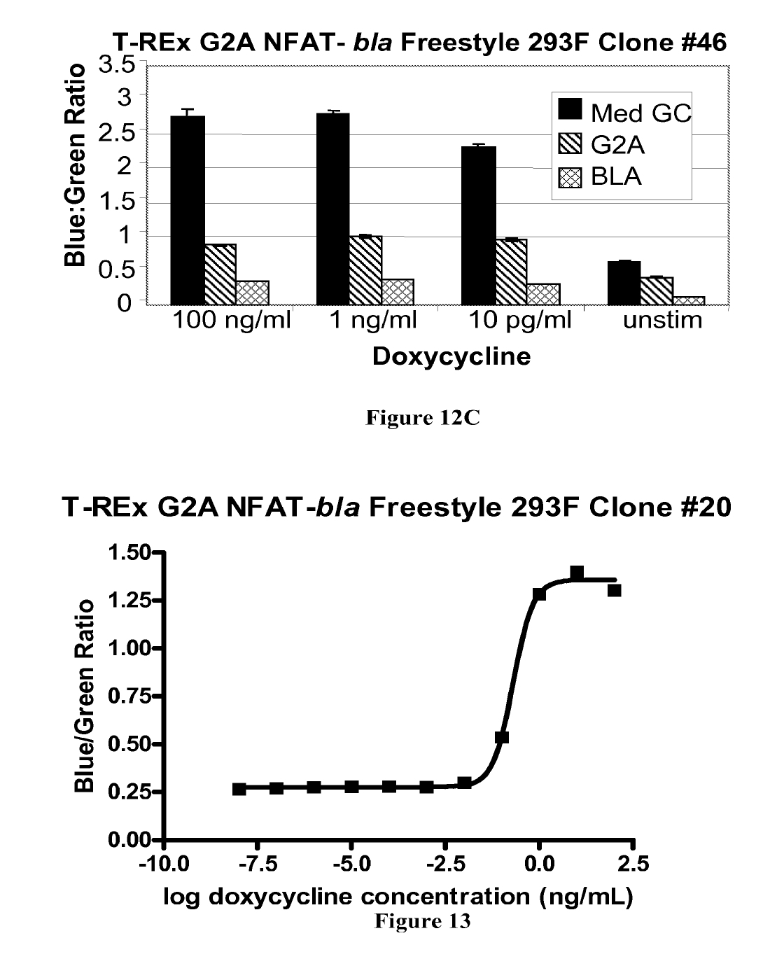

Data is shown in FIG. 12. In all three clones, siRNAs directed against either G2A or bla strongly knock down both the doxycycline-induced and the background beta-lactamase signal relative to levels seen with the Med GC negative control siRNA. This confirms that the signaling seen in these cells is G2A dependent.

### Example 10

### Clone Confirmation

Clone #20 was selected for final experiments. One dose response was performed to confirm doxycycline activation.

For this confirmation, cells were plated at 10,000 cells/well for ˜5 hours prior to stimulation with doxycycline for 16 h. Following induction, the cells were loaded with LiveBlazer™-FRET B/G for 2 h and the beta-lactamase response measured (FIG. 13). This clone was selected based on the expectation of superior response characteristics and that expectation was fulfilled by the measured responses, all improved relative to the original pool.

### Example 11

### Cell Line Validation

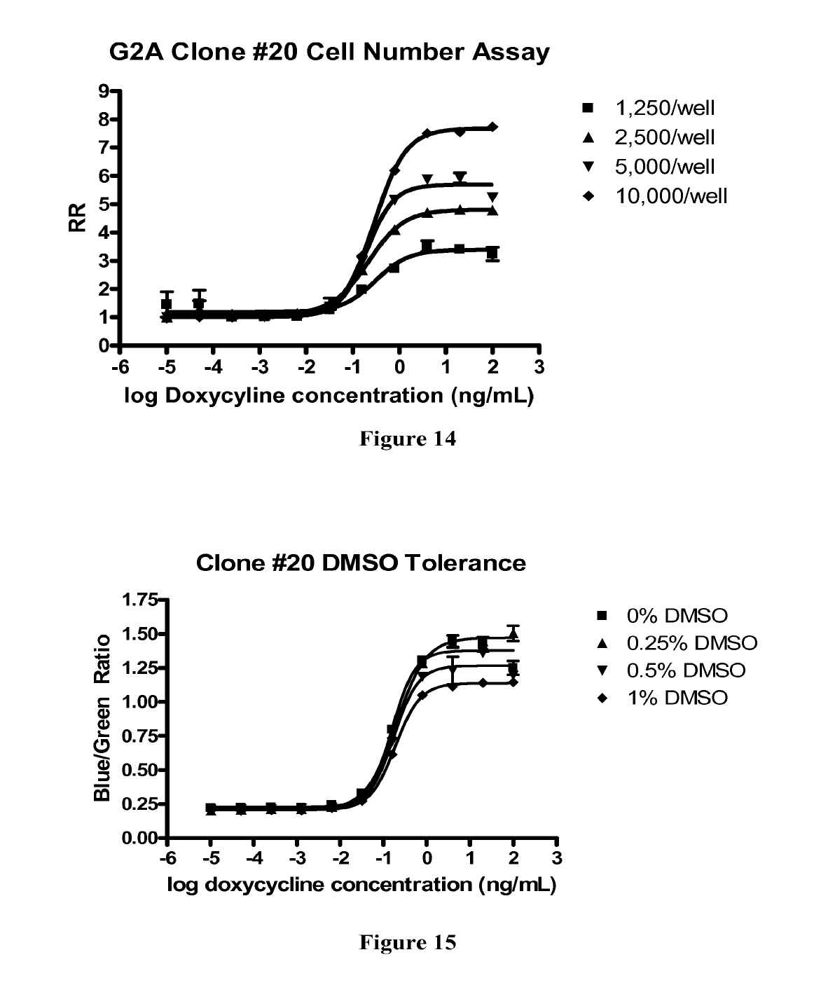

Cell Density/well—The influence of cell-plating density on assay performance was tested to find the density giving the best response and Z′ while maintaining the expected pharmacology. Cells were plated 24 hours prior to the assay at the densities indicated in FIG. 14 in DMEM+10% dFBS and incubated at 37° C./5% CO2 until the time of assay. The cells were stimulated for 16 hours at 37° C./5% CO2 with a dilution series of the doxycycline. After incubation, the cells were loaded for 2 hours at room temperature in the dark with LiveBLAzer™-FRET B/G loading solution containing 1 μM substrate. This assay was performed in the presence of 0.5% DMSO to simulate the effect that solvents used in a compound library may have on the assay. Based upon the EC₅₀, response ratio, and Z′ values determined (see Table 4), 10,000 cells/well was chosen (plated one day prior to the assay) as the cell density to be used for further validation experiments. Results are shown in FIG. 14.

TABLE 5

| | | RR | Z′ |
|---|---|---|---|
| Cells/Well | EC₅₀ | (Max) | (Max) |
| 1,250 | 305.5 pg/mL | 3.51 | 0.16 |
| 2,500 | 215.6 pg/mL | 4.8 | 0.73 |
| 5,000 | 198.6 pg/mL | 5.93 | 0.83 |
| 10,000 | 290.9 pg/mL | 7.7 | 0.78 |

DMSO Tolerance—Tests were performed related to assay performance as a function of DMSO concentration to determine the robustness of the assay to the range of DMSO challenge routinely encountered during high-throughput screening. Dose response curves were performed in the presence of 0%, 0.25%, 0.5%, or 1% DMSO. Cells were plated at 10,000 cells/well 24 hours prior to assay in DMEM+10% dFBS and incubated at 37° C./5% CO2 until time of assay. After incubation, DMSO was added to the wells to final concentrations of 0%, 0.25%, 0.5% and 1% DMSO. The cells were stimulated for 16 hours with a dilution series of the doxycycline. Following stimulation, the cells were loaded for 2 hours at room temperature in the dark with LiveBLAzer™-FRET B/G loading solution containing 1 μM substrate.

The assay was tolerant to at least 1% DMSO. Acceptable EC₅₀, response ratio and Z′ performance was observed across the range of DMSO tested (Table 5), although at 1% DMSO, a drop in the response ratio was observed. Results are shown in FIG. 15.

TABLE 5

| | | RR | Z′ |
|---|---|---|---|
| % DMSO | EC₅₀ | (Max) | (Max) |
| 0.0 | 159.2 pg/mL | 6.9 | 0.74 |
| 0.25 | 197.3 pg/mL | 7.2 | 0.59 |
| 0.50 | 172.5 pg/mL | 6.5 | 0.86 |
| 1.00 | 187.4 pg/mL | 5.4 | 0.76 |

The substrate loading times were varied to find the time for an optimal assay performance. Cells were plated at 10,000 cells/well 24 hours prior to assay in DMEM+10% dFBS and incubated at 37° C./5% CO2 until time of assay. After incubation, DMSO was added to the wells to final concentration of 0.5%. The cells were stimulated for 16 hours with a dilution series of the doxycycline. Following stimulation, the cells were loaded for 1, 1.5, or 2 hours at room temperature in the dark with LiveBLAzer™-FRET B/G loading solution containing 1 μM substrate.

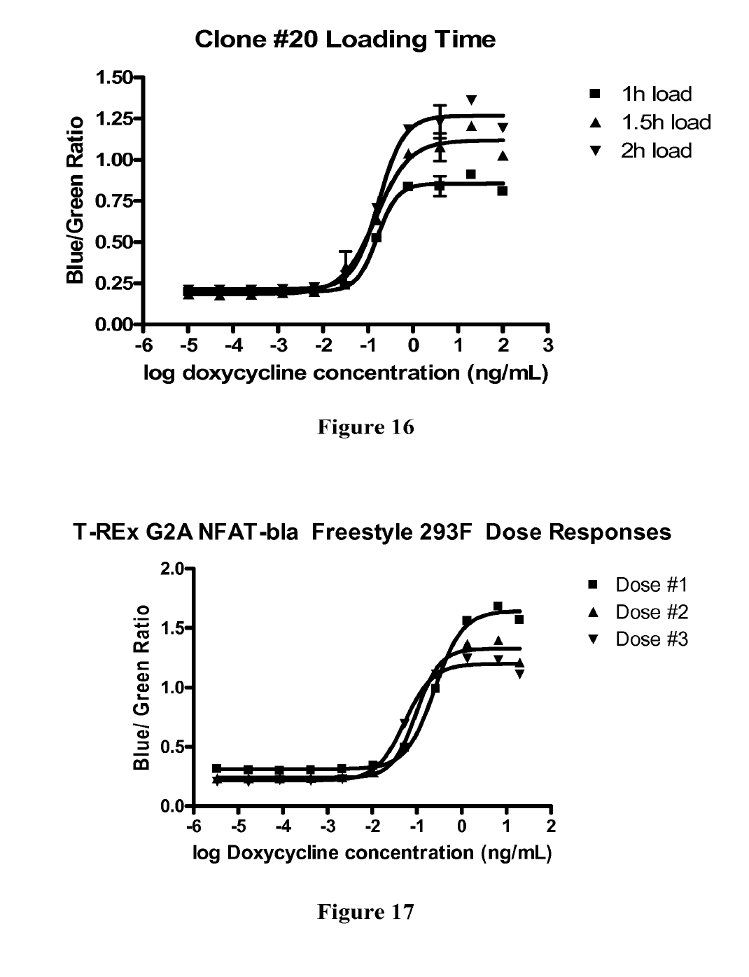

This assay may be performed with a 90 to 120 minute loading time without a significant drop in RR or Z′ (Table 6). All validation experiments were done with a 2 h loading time. Results are shown in FIG. 16.

TABLE 6

| Loading | | RR | Z′ |
|---|---|---|---|
| Time | EC₅₀ | (Max) | (Max) |
| 60 min | 161.6 pg/mL | 4.5 | 0.79 |
| 90 min | 151 pg/mL | 6.5 | 0.84 |
| 120 min | 172.5 pg/mL | 6.5 | 0.85 |

Assay Reproducibility—To assess the assay reproducibility under the optimized conditions and to determine the average EC₅₀, Z′, and response ratio values, a dose response assay was repeated on three separate days. The cells were plated at 10,000 cells/well 24 hours prior to assay in DMEM+10% dFBS and incubated at 37° C./5% CO2 until time of assay. The cells were stimulated for 16 hours with a dilution series of the doxycycline at 37° C./5% CO2. Following stimulation, the cells were loaded 2 hours at room temperature in the dark with LiveBLAzer™-FRET B/G loading solution containing 1 μM substrate. Cells were assayed in the presence of 0.5% DMSO. Each assay gave very similar Response Ratios and acceptable Z prime values (Table 7). EC₅₀'s fell within a 5-fold range of one another. Results are shown in FIG. 17.

TABLE 7

| | EC₅₀ | RR (Max) | Z′ (Max) |
|---|---|---|---|
| DR 1 | 245.7 pg/mL | 5.36 | 0.58 |
| DR 2 | 96.3 pg/mL | 5.95 | 0.63 |
| DR 3 | 53.3 pg/mL | 5.99 | 0.80 |
| Average | 131.8 pg/mL | 5.77 | 0.71 |

Assay Reliability—To assess assay reliability, ten 384 well plates were manually-plated 24 hours prior to assay at 10,000 cells/well in DMEM+10% dFBS and incubated at 37° C./5% CO2 until time of assay. This assay was also repeated on two separate days. One half of the plate was stimulated with 20 ng/mL doxycycline while the other half was left unstimulated. The plate was incubated for 16 hours at 37° C./5% CO2. Following stimulation, the cells were loaded 2 hours at room temperature in the dark with LiveBLAzer™-FRET B/G loading solution containing 1 μM substrate. Results are shown in Table 7. Both assays performed as expected with all plates showing within-plate Z′ greater than 0.5, as well as interplate Z′ in excess of 0.5.

TABLE 7

| | |
|---|---|
| Assay 1 | Assay 2 |

| Plate # | Z′ | Plate # | Z′ |
|---|---|---|---|
| 1 | 0.73 | 1 | 0.70 |
| 2 | 0.64 | 2 | 0.75 |
| 3 | 0.63 | 3 | 0.73 |
| 4 | 0.68 | 4 | 0.76 |
| 5 | 0.68 | 5 | 0.77 |
| 6 | 0.71 | 6 | 0.70 |
| 7 | 0.69 | 7 | 0.67 |
| 8 | 0.68 | 8 | 0.71 |
| 9 | 0.71 | 9 | 0.69 |
| 10 | 0.64 | 10 | 0.69 |
| Interplate | 0.50 | Interplate | 0.68 |

Plating from a Frozen Vial—To determine whether frozen cells could be thawed and used immediately in the assay versus first undergoing a recovery period in culture before use, the assay was run in dose response format using doxycycline induction on cells taken from culture versus frozen cells that were thawed immediately before use. The frozen cells were thawed, brought up in 10 mls DMEM+10% dFBS, counted, and centrifuged to remove DMSO. The cell pellet was re-suspended in the calculated volume of media to have 10,000 cells/well and plated. Cells were incubated at 37° C./5% CO2 for 24 hours. The cells were stimulated with a dilution series of doxycycline for 16 hours at 37° C./5% CO₂. Following stimulation, the cells were loaded 2 hours at room temperature in the dark with LiveBLAzer™-FRET B/G loading solution containing 1 μM substrate.

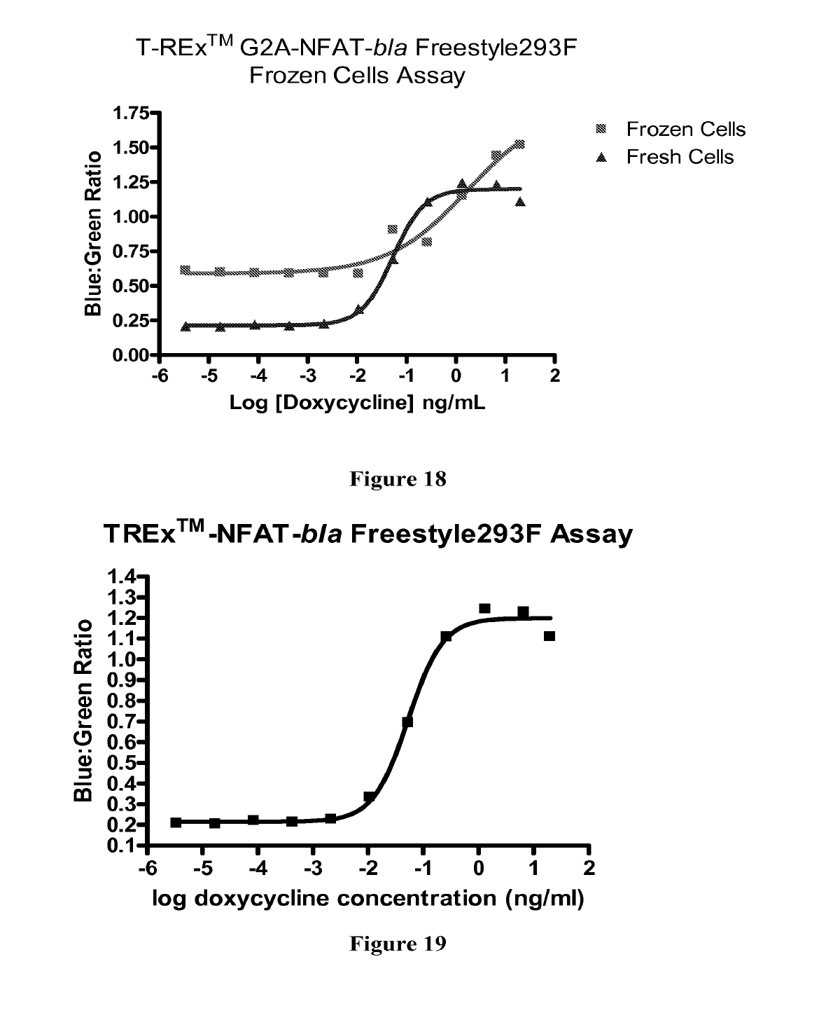

The freshly thawed cells did not perform as well as cells which have been passed a few times (Table 8). Results are shown in FIG. 18. In general, Freestyle™ 293F cells require some recovery time after thawing, so this result is not unexpected.

TABLE 8

| | EC₅₀ | RR (Max) | Z′ (Max) |
|---|---|---|---|
| Frozen | >985 pg/mL | 2.48 | 0.5 |
| Fresh | 53.3 pg/mL | 5.99 | 0.80 |

Summary—The T-REx™ G2A NFAT-bla Freestyle™ 293F cells were validated The assay is robust and suitable for high-throughput screening

### Example 12

### An Exemplary Assay Protocol

This example provides exemplary protocols of the invention. Other exemplary assay protocols are provided herein, e.g., see Example 21. Further, the methods described herein are set out in a format which is conducive for insertion into product literature of the invention.

The exemplary assay protocol below uses T-REx™ G2A NFAT-bla Freestyle™ 293F cells as an exemplary cell line. However, very similar and even the same protocol can be utilized with other cell lines. One skilled in the art can determine how to use the exemplary assay protocol herein with other cell lines.

Some embodiments of the invention provide an assay protocol for a TREx™-mG2A-NFAT-bla FreeStyle™ 293F similar to the one below. In some of these and other embodiments, the assay medium of Table 10 also comprises 25 mM Hepes (pH 7.3). In some embodiments related to mG2A, Section 6.3 below would be replaced with the following paragraph.

TREx™-mG2A-NFAT-bla Freestyle293F cells were stimulated for 16 hours with doxycycline in the presence of 0.5% DMSO. Cells were then loaded with LiveBLAzer™-FRET B/G (CCF4-AM) for 2 hours. Fluorescence emission values at 460 nm and 530 nm are obtained using a standard florescence plate reader and the Blue/Green Emission ratios are plotted against the concentration of the stimulant. Results are shown in FIG. 29 and produced an EC₅₀ for clone #2 of 386 pg/ml; for clone #25 of 1.12 ng/ml; for and clone #53 of 524 pg/ml.

Table Of Contents

| | |
|---|---|
| 1.0 | Overview Of Geneblazer ® Reporter Technology |
| 2.0 | Materials Supplied |
| 3.0 | Materials Required, But Not Supplied |
| 3.1 | Optional Equipment And Materials |
| 4.0 | Cell Culture Conditions |
| 4.1 | Media Required |
| 4.2 | Growth Conditions |
| 5.0 | Assay Procedure |
| 5.1 | Quick Assay Reference Guide |
| 5.2 | Detailed Assay Protocol |
| 5.3 | Detection |
| 6.0 | Data Analysis |
| 6.1 | Background Subtraction And Ratio Calculation |
| 6.2 | Visual Observation Of Intracellular Beta-Lactamase Activity Using |
| | Liveblazer ™ - FRET B/G Substrate (CCF4-Am) |
| 6.3 | Representative Data |
| 7.0 | Appendix A (Detailed Cell Handling Protocol) |
| 7.1 | Thawing Method |
| 7.2 | Propagation Method |
| 7.3 | Freezing Method |
| 8.0 | References |
| 1.0 | Overview Of Geneblazer ® Beta-Lactamase Reporter Technology |

GeneBLAzer® Beta-lactamase Reporter Technology provides a highly accurate, sensitive and easy to use method of monitoring cellular response to drug candidates or other stimuli (1). The core of the GeneBLAzer® Technology is a Förster Resonance Energy Transfer (FRET) substrate that generates a ratiometric reporter response with minimal experimental noise. In addition to the dual-color (blue/green) readout of stimulated and unstimulated cells, this ratiometric method reduces the absolute and relative errors which can mask the underlying biological response of interest. Such errors include variations in cell number, transfection efficiency, substrate concentration, excitation path length, fluorescence detectors and volume changes. The GeneBLAzer® Beta-lactamase Reporter Technology has been proven in high-throughput screening campaigns for a range of target classes, including G-protein coupled receptors (2, 3), nuclear receptors (4-6) and kinase signaling pathways (7).

### 2.0 MATERIALS SUPPLIED

### Cell Line Name: T-REx™-G2A-NFAT-bla FreeStyle™ 293F

Description: The GeneBLAzer® T-REx™-G2A-NFAT-bla FreeStyle™ 293F cells contain a beta-lactamase reporter gene under control of the NFAT response element stably integrated into Freestyle™293F cells. This cell line also stably expresses the G2A receptor under control of the Tet-Operon and the tetracycline repressor protein. This cell line can be used to detect agonists/inverse agonists of the G2A receptor. T-REx™-G2A-NFAT-bla FreeStyle™ 293F cells exhibit constitutive beta-lactamase activity when G2A expression is induced with doxycycline or tetracycline.

### Shipping Condition: Dry Ice

Storage Condition: Liquid Nitrogen. Immediately upon receipt, cells should be stored in liquid nitrogen. Cells should not be stored at −80° C., as they can quickly lose viability.
Quantity: ˜2,000,000 cells (2×10⁶ cells/ml)
Application: This cell line can be used to detect agonists/inverse agonists of G2A.

### Growth Properties: Adherent

### Cell Phenotype: Epithelial

Selection Marker(s): Zeocin™ (100 μg/ml), Hygromycin (40 μg/ml), Blasticidin (5 μg/ml)

### Mycoplasma Testing: Negative

### BioSafety Level: 2

### 3.0 Materials

TABLE 9

| | Recommended | |
|---|---|---|
| Media/Reagents | Source | Catalog # |
| LiveBLAzer ™ Loading Kit | Invitrogen | K1030 |
| LiveBLAzer ™-FRET B/G Substrate | | Other sizes or |
| (CCF4-AM) substrate (5 mg) | | Loading Kits |
| DMSO for Solution A | | are available |
| Solution B | | |
| Solution C | | |
| Cell Culture Freezing Medium | Invitrogen | 11101-011 |
| DMEM (high-glucose), glutaMAX | Invitrogen | 10569-010 |
| DMSO | Fluka | 41647 |
| Fetal bovine serum, (FBS), dialyzed, | Invitrogen | 26400-036 |
| tissue-culture grade | | |
| Non-essential amino acids (NEAA) | Invitrogen | 11140-050 |
| Penicillin/Streptomycin | Invitrogen | 15140-122 |
| Phosphate-buffered saline without | Invitrogen | 14190-136 |
| calcium and magnesium [PBS(−)] | | |
| HEPES (1M, pH 7.3) | Invitrogen | 15630-80 |
| Sodium pyruvate | Invitrogen | 11360-070 |
| Doxycycline | MP Biomedical | 2195044.2 |
| 0.05% Trypsin/EDTA | Invitrogen | 25300-054 |
| Blasticidin antibiotic | Invitrogen | R210-01 |
| Hygromycin antibiotic | Invitrogen | 10687-010 |
| Zeocin ™ antibiotic | Invitrogen | R250-01 |
| | Recommended | |
| Consumables | Source | |
| Poly-D Lysine coated, Black-wall, | BD Biosciences | 354663 |
| clear-bottom, 384-well assay plates | | |
| Compressed Air | Various | — |

| Equipment | Recommended Source |
|---|---|
| Fluorescence plate reader with | Various |
| bottom-read capabilities | |
| Filters if required for plate reader | Chroma Technologies |
| (see Section 5.3.1) | |

### 3.1 Optional Equipment and Materials

Epifluorescence or fluorescence-equipped microscope, with appropriate filters
Microplate centrifuge

### 4.0 CELL CULTURE CONDITIONS

TABLE 10

| | Growth | Growth | | |
|---|---|---|---|---|
| | Medium | Medium | Assay | Freezing |
| Component | (−) | (+) | Medium | Medium |
| DMEM | 90% | 90% | 90% | — |
| Dialyzed FBS | 10% | 10% | 10% | — |
| NEAA | 0.1 mM | 0.1 mM | 0.1 mM | — |
| HEPES (pH 7.3) | 25 mM | 25 mM | — | — |
| Penicillin | — | 100 U/ml | 100 U/ml | — |
| Streptomycin | — | 100 μg/ml | 100 □g/ml | — |
| Blasticidin antibiotic | — | 5 μg/ml | — | — |
| Hygromycin antibiotic | — | 40 μg/ml | — | — |
| Zeocin ™ | — | 100 μg/ml | — | — |
| Recovery ™ Cell Culture | — | — | — | 100% |
| Freezing Medium | | | | |

Note:
Unless otherwise stated, all media and solutions should be at least at room temperature (37° C. is best) before adding them to the cells.

### 4.2 Growth Conditions

1. Cells should be thawed in Growth Medium (−) and grown in Growth Medium (+). Cells should be passaged or fed at least twice a week and maintained in a 37° C./5% CO-2 incubator. Cells should be maintained between 10% and 90% confluence. Do should not be allowed to reach confluence.

2. Cells should be frozen at 2×10⁶ cells/ml in Freeze Medium.

3. For detailed growth and maintenance directions see Appendix A.

### 5.0 ASSAY PROCEDURE

The following instructions outline the recommended procedure for determining activity of compounds as modulators of G2A receptor using beta-lactamase as the readout.

### 5.1 Quick Assay Reference Guides (for More Detailed Protocol, See Section 5.2).

TABLE 11
Agonist Assay Quick Reference Guide

| | | | | Test |
|---|---|---|---|---|
| | Unstimulated | | Cell-free | Compound |
| | Wells | Stimulated Wells | Wells | Wells |
| Step 1 | 32 μl cells in Assay | 32 μl cells in Assay | 32 μl Assay | 32 μl cells in |
| Plating Cells | Medium | Medium | Medium | Assay |
| | (10,000 cells/well) | (10,000 cells/well) | (no cells) | Medium |
| | | | | (10,000 |
| | | | | cells/well) |
| Step 2 | Incubate the plate in the 37° C. incubator for 24 hours. | | | |
| Incubation | | | | |
| Step 3 | 8 μl Assay Medium | 8 μl 5X | 8 μl 5X | 8 μl 5X Test |
| Adding | with 2.5% DMSO. | Doxycycline in | Doxycycline in | Compounds in |
| Agonist or | | Assay Medium | Assay Medium with | 2.5% DMSO. |
| Test | | with 2.5% DMSO. | 2.5% DMSO. | |
| Compounds | | | | |

| | |
|---|---|
| Step 4 | Incubate the plate in the 37° C. incubator for 5-16 hours. (Note: G2A is an |
| Incubation | orphan GPCR. Stimulation time with test compounds needs to be determined |
| | empirically. A 16 hour stimulation with doxycycline leads to optimal G2A |
| | constitutive activity for the control; however in general, 5 hour stimulation |
| | times with test compounds result in optimal reporter gene activity for other |
| | GPCRs.) |
| Step 5 | 6 μl of 1 mM LiveBLAzer ™-FRET B/G (CCF4-AM) substrate + 60 μl of |
| Prepare 6X | solution B, mix + |
| Loading | 934 μl of Solution C, mix |
| Solution | |
| Step 6 | 8 μl per well |
| Substrate | |
| Loading | |
| Step 7 | 2 hrs at Room Temperature in the dark |
| Substrate | |
| Incubation | |
| Step 8 | See Section 5.3 |
| Detection | |
| Step 9 | See Section 6.0 |
| Data | |
| Analysis | |

TABLE 12
Inverse Agonist Assay Quick Reference Guide

| | Unstimulated | Stimulated | Inverse Agonist | Cell-free | Test Compound |
|---|---|---|---|---|---|
| | Wells | Wells | Control Wells | Wells | Wells |
| Step 1 | 32 μl cells in | 32 μl cells in | 32 μl cells in | 32 μl Assay | 32 μl cells in |
| Plating Cells | Assay Medium | Assay Medium | Assay Medium | Medium | Assay Medium |
| | (10,000 | (10,000 | (10,000 | (no cells) | (10,000 |
| | cells/well) | cells/well) | cells/well) | | cells/well) |
| Step 2 | Incubate the plate in the 37° C. incubator for 24 hours. | | | | |
| Incubation | | | | | |
| Step 3 | 4 μl Assay | 4 μl | 4 μl | 4 μl | 4 μl 10X |
| Inducing | Medium with | Doxycycline | Doxycycline in | Doxycycline | Doxycycline in |
| Constitutive | 2.5% DMSO. | in Assay | Assay Medium | in Assay | Assay Medium |
| Expression | | Medium with | with 2.5% | Medium | with 2.5% |
| | | 2.5% DMSO. | DMSO. | with 2.5% | DMSO. |
| | | | | DMSO. | |
| Step 4 | Incubate the plate in the 37° C. incubator for 16 hours. | | | | |
| Incubation | | | | | |
| Step 5 | 4 μl Assay | 4 μl Assay | 4 μl 10X Inverse | 4 μl Assay | 4 μl 10X Test |
| Adding | Medium with | Medium with | Agonist in | Medium | Cmpds in Assay |
| Inverse | 2.5% DMSO. | 2.5% DMSO. | Assay Medium | with 2.5% | Medium with |
| Agonist | | | with 2.5% | DMSO. | 2.5% DMSO. |
| | | | DMSO. | | |

| | |
|---|---|
| Step 6 | Incubate the plate in the 37° C. incubator for 5 hours. |
| Incubation | |
| Step 7 | 6 μl of 1 mM LiveBLAzer ™-FRET B/G (CCF4-AM) substrate + 60 μl of |
| Prepare 6X | solution B, mix + |
| Loading | 934 μl of Solution C, mix |
| Solution | |
| Step 8 | 8 μl per well |
| Substrate | |
| Loading | |
| Step 9 | 2 hrs at Room Temperature in the dark |
| Substrate | |
| Incubation | |
| Step 10 | See Section 5.3 |
| Detection | |
| Step 9 | See Section 6.0 |
| Data Analysis | |

### 5.2 Detailed Assay Protocol

Plate layouts and experimental outlines will vary; in screening mode, it is recommended to use at least three wells for each control: Unstimulated Control, Stimulated Control, and Cell-Free Control.

Note: Certain solvents may affect assay performance. The effect of solvent should be assessed prior to screening. The cell stimulation described below is carried out in the presence of 0.5% DMSO to simulate the effect that the test compound solvent may have on the assay. If other solvents and/or solvent concentrations are used, the following assay should be changed accordingly.

### 5.2.1 Precautions

1. Work on a dust-free, clean surface. Always handle the 384-well, black-wall, clear-bottom assay plate by the sides; do not touch the clear bottom of the assay plate.

2. If pipetting manually, it may be necessary to centrifuge the plate briefly at room temperature (for 1 min. at 14×g) after additions to ensure all the assay components are on the bottom of the wells.

### 5.2.2 Plating Cells

### Day 1:

Note: For optimal assay performance, the assay should be performed in Poly-D Lysine coated pates.

1. Harvest cells and resuspend in Assay Medium at a density of 3.125×10⁵ cells/ml.

2. Add 32 μl per well of the Assay Medium to the Cell-Free Control wells. Add 32 μl per well of the cell suspension to the Test Compound wells, the Unstimulated Control wells, and Stimulated Control wells.

3. Incubate the assay plate in a humidified 37° C./5% CO2 incubator for 24 hours.

### Day 2:

### 5.2.4 Agonist Assay Plate Setup

Note: This subsection provides directions for performing an agonist assay. Directions for performing an inverse agonist assay can be found in Section 5.2.5. G2A is an orphan GPCR. Stimulation time with test compounds needs to be determined empirically. A 16 hour stimulation with doxycycline leads to optimal G2A constitutive activity for the control; however in general, 5 hour stimulation times with test compounds result in optimal reporter gene activity for other GPCRs.

1. Prepare a stock solution of 2.5% DMSO in Assay Medium.

2. Prepare a 5× stock of test compounds in Assay Medium with 2.5% DMSO.

3. Prepare a 5× stock of doxycycline in Assay Medium with 2.5% DMSO. We recommend running a dose response curve to determine the optimal concentration for your doxycycline solution. In some cases, 7 ng/ml final concentration (5×=70 ng/ml) may be used

4. Add 8 μl of the stock solution of 2.5% DMSO in Assay Medium to the Unstimulated Control wells.

5. Add 8 μl of the 5× stock solution of doxycycline to the Stimulated Control wells and the Cell-Free Control wells.

6. Add 8 μl of the 5× stock of Test Compounds to the Test Compound wells.

7. Incubate the agonist assay plate in a humidified 37° C./5% CO2 incubator for 16 hours.

### 5.2.5 Inverse Agonist Assay Plate Setup

Note: This subsection provides directions for performing an inverse agonist assay. Directions for performing an agonist assay are provided in Section 5.2.4. G2A is an orphan GPCR. Stimulation time with test compounds needs to be determined empirically. A 16 hour stimulation with doxycycline leads to optimal G2A constitutive activity and in general, hour stimulation times with test compounds result in optimal reporter gene activity for other GPCRs.

1. Prepare a stock solution of 2.5% DMSO in Assay Medium.

2. Prepare a 10× stock of test compounds in assay medium with 2.5% DMSO.

3. Prepare a 10× stock of doxycycline in Assay Medium with 2.5% DMSO. It is recommended to run a dose response curve to determine the EC80 for your doxycycline solution. In some cases, ˜130 pg/ml final concentration (10×=1.3 ng/ml) may be used, e.g., for an EC₅₀ concentration.

4. Prepare a 10× stock of a known inverse agonist (if available) in assay medium with 2.5% DMSO. It is recommended to run a dose response curve to determine the optimal inhibition concentration for your inverse agonist solution.

5. Add 4 μl of the 10× stock of doxycycline to the Test Compound wells, the Cell-free control wells, the Inverse Agonist control wells, and the Stimulated Control wells.

6. Add 4 μl of the stock solution of 2.5% DMSO to the Unstimulated Control wells.

7. Incubate the assay plate in a humidified 37° C./5% CO2 incubator for 16 hours.

8. Add 4 μl of the stock solution of 2.5% DMSO to the Unstimulated Control wells, the Stimulated Control wells, and the Cell-Free Control wells.

9. Add 4 μl of the 10× stock of the known inverse agonist in Assay Medium with 2.5% DMSO to the Inverse Agonist Control wells.

10. Add 4 μl of the 10× stock of test compounds to the Test Compound wells.

11. Incubate the assay plate in a humidified 37° C./5% CO2 incubator for 5 hours.

### 5.2.6 Substrate Loading and Incubation

This protocol is designed for loading cells with LiveBLAzer™-FRET B/G Substrate (CCF4-AM) or CCF2-AM. If alternative substrates are used follow the loading protocol provided with the substrate.

Preparation of LiveBLAzer™-FRET B/G Substrate (CCF4-AM) or CCF2-AM Loading Solution and cell loading should be done in the absence of direct strong lighting. Turn off the light in the hood.

1. Prepare Solution A: 1 mM LiveBLAzer™-FRET B/G Substrate (CCF4-AM) stock solution in dry DMSO. Store the aliquots of the stock solution at −20° C. until use. The molecular weight of CCF2-AM is 1082 g/mol, and the molecular weight of the LiveBLAzer™-FRET B/G Substrate (CCF4-AM) is 1096 g/mol.

2. Prepare 6× Loading Solution:
Add 6 μl of Solution A to 60 μl of Solution B and vortex.
Add 934 μl Solution C to the above solution and vortex.

3. Remove assay plate from the incubator.

4. Add 8 μl of the 6× Loading Solution to each well.

5. Cover the plate to protect it from light and evaporation.

6. Incubate at room temperature for 120 minutes.

Note: Handle the plate gently and do not touch the bottom.

### 5.3 Detection

All measurements are made at room temperature from the bottom of the wells, for example in Poly-D lysine coated, 384-well, black-wall, clear-bottom assay plates with low fluorescence background. Before reading the plate, remove dust from the bottom with compressed air.

### 5.3.1 Instrumentation, Filters, and Plates

Fluorescence plate reader with bottom reading capabilities.
Recommended filters for fluorescence plate reader:
   Excitation filter: 409/20 nm
   Excitation filter: 460/40 nm
   Excitation filter: 530/30 nm

### 5.3.2 Reading an Assay Plate

1. Set the fluorescence plate reader to bottom-read mode.

2. Allow the lamp in the fluorescence plate reader to warm up for at least 10 min. before making measurements.

3. Use the following filter selections:

TABLE 13

| | Scan 1 | Scan 2 |
|---|---|---|
| Purpose: | Measure fluorescence in the blue | Measure FRET signal in the |
| | channel | green channel |
| Excitation | 409/20 nm | 409/20 nm |
| filter: | | |
| Emission | 460/40 nm | 530/30 nm |
| filter: | | |

### 6.0 DATA ANALYSIS

### 6.1 Background Subtraction and Ratio Calculation

Background subtraction for both emission channels (460 nm and 530 nm) is recommended.

1. Use the assay plate layout to identify the location of the Cell-Free Control wells. These control wells are used for background subtraction.

2. Determine the average emission from the Cell-Free Control wells at both 460 nm (Average Blue Background) and 530 nm (Average Green Background).

3. Subtract the Average Blue background from all of the Blue emission data.

4. Subtract the Average Green background from all of the Green emission data.

5. Calculate the Blue/Green Emission Ratio for each well, by dividing the background subtracted blue emission values by the background subtracted green emission values.

### 6.2 Visual Observation of Intracellular Beta-Lactamase Activity Using LiveBLAzer™-FRET B/G Substrate (CCF4-AM)

Note: Microscopic visualization of cells will cause photobleaching. Always read the assay plate in the fluorescence plate reader before performing microscopic visualization. An inverted microscope equipped for epifluorescence and either a xenon or mercury excitation lamp may be used to view the LiveBLAzer™-FRET B/G Substrate (CCF4-AM) signal in cells. To visually inspect the cells, you may want a long-pass filter passing blue and green fluorescence light, so that your eye may visually identify whether the cells are fluorescing green or blue. Recommended filter sets for observing beta-lactamase activity are described below and are available from Chroma Technologies (800-824-7662) www.chroma.com.

### Chroma Set # 41031

Excitation filter: HQ405/20x (405±10)
Dichroic mirror: 425 DCXR
Emission filter: HQ435LP (435 long-pass)

Filter sizes vary for specific microscopes and need to be specified when the filters are ordered. For epifluorescence microscopes, a long-pass dichroic mirror can be used to separate excitation and emission light and should be matched to the excitation filter (to maximally block the excitation light around 405 nm, yet allow good transmission of the emitted light).

### 6.3 Representative Data

TABLE 14

| EC₅₀ | 0.05 ng/ml |
|---|---|
| EC₁₀₀ | 2.2 ng/ml |
| Z′ at EC₁₀₀ | 0.80 |

T-REx™-NFAT-bla Freestyle™293F cells were stimulated for 16 hours with doxycycline in the presence of 0.5% DMSO. Cells were then loaded with LiveBLAzer™-FRET B/G (CCF4-AM) for 2 hours. Florescence emission values at 460 nm and 530 nm are obtained using a standard florescence plate reader and the Blue/Green Emission ratios are plotted against the concentration of the stimulant. See FIG. 19

### 7.0 APPENDIX A (DETAILED CELL HANDLING PROTOCOL)

### 7.1 Thawing Method

1. Place 14 ml of Growth Medium (−) into a T75 flask.

2. Place the flask in a 37° C./5% CO2 incubator for 15 minutes to allow medium to equilibrate to the proper pH and temperature.

3. Remove vial of cells to be thawed from liquid nitrogen and rapidly thaw by placing at 37° C. in a water bath with gentle agitation for 1-2 minutes. Do not submerge vial in water.

4. Decontaminate the vial by wiping with 70% ethanol before opening in a Class II biological safety cabinet.

5. Transfer the vial contents dropwise into 10 ml of Growth Medium (−) in a sterile 15 ml conical tube.

6. Centrifuge cells at 200×g for 5 minutes.

7. Aspirate supernatant and resuspend the cell pellet in 1 ml of fresh Growth Medium

8. Transfer contents to the T75 tissue culture flask containing pre-equilibrated Growth Medium (−) and place flask in the 37° C./5% CO2 incubator.

9. At first passage switch to Growth Medium (+).

### 7.2 Propagation Method

1. Cells should be passaged or fed at least twice a week. Cells should be maintained between 10% and 90% confluence. Cells should not be allowed to reach confluence.

2. To passage cells, aspirate medium, rinse once in PBS, add Trypsin/EDTA (3 ml for a T75 flask and 5 ml for a T175 flask and 7 ml for T225 flask) and swirl to coat the cells evenly. Cells usually detach after 2-5 minutes exposure to Trypsin/EDTA. Add an equal volume of Growth Medium to inactivate Trypsin.
Verify under a microscope that cells have detached and clumps have completely dispersed.
Centrifuge cells at 200×g for 5 minutes and resuspend in Growth Medium (+).

### 7.3 Freezing Method

1. Harvest the cells as described in Section 7.2, Step 2. After detachment, count the cells, then spin cells down and resuspend in 4° C. Cell Culture Freezing Medium at 2×10⁶ cells/ml.

2. Dispense 1.0 ml aliquots into cryogenic vials.

3. Place in an insulated container for slow cooling and store overnight at −80° C.

4. Transfer to liquid nitrogen the next day for storage.

### 8.0 REFERENCES

1. Zlokarnik, G., et al, Quantitation of Transcription and Clonal Selection of Single Living Cells with β-Lactamase as Reporter, (1998) Science; 279: p84-88.
2. Kunapuli P, Ransom R, Murphy K, Pettibone D, Kerby J, Grimwood S, Zuck P, Hodder P, Lacson R, Hoffman I, Inglese J, Strulovici B, Development of an Intact Cell Reporter Gene β-lactamase Assay for G Protein-coupled Receptors, (2003) Analytical Biochem.; 314: p16-29.
3. Xing, H., Pollok, B., et al, A Fluorescent Reporter Assay For The Detection of Ligands Acting Through G1 Protein-coupled Receptors, (2000) J. Receptor & Signal Transduction Research; 20: p189-210.
4. Qureshi, S., et al, A One-Arm Homologous Recombination Approach for Developing Nuclear Receptor Assays in Somatic Cells, (2003) Assay and Drug Dev. Tech; 1: p755-766.
5. Peekhaus, N. et al, A β-Lactamase-Dependent Ga14-Estrogen Receptor Transactivation Assay for the Ultra-High Throughput Screening of Estrogen Receptor Agonists in a 3,456-Well Format, (2003) Assay and Drug Dev Tech; 1: p789-800.
6. Chin, J., et al, Miniaturization of Cell-Based β-Lactamase-Dependent FRET Assays to Ultra-High Throughput Formats to Identify Agonists of Human Liver X Receptors, (2003) Assay and Drug Dev. Tech.; 1: p777-787.
7. Whitney M, Rockenstein E, Cantin G, Knapp T, Zlokarnik G, Sanders P, Durick K, Craig F F, Negulescu P A., A Genome-wide Functional Assay of Signal Transduction in Living Mammalian Cells, (1998) Nat. Biotechnol.; 16: p1329-1333.

### Example 13

### Expressing GPCRS in an Active State in the Absence of their Ligand

An expression construct for a signaling receptor is introduced via transfection with Lipofectamine 2000 (Invitrogen Cat#11668-019) or transduction using the ViraPower Lentiviral system (Invitrogen Cat#K4970-00) into a cell line (e.g., CHO-k1 (ADORA2A, PDR, PE2R, M4, M1, M3, M5), HEK293T (CCKBR), ERalpha (Griptite HEK293)). The expression plasmid uses a constitutively active promoter (CMV). The cells comprise a nucleic acid comprised of a beta-lactamase coding region controlled by a promoter that is responsive to changes in cAMP, calcium levels, and/or ga14 binding. The cells are then selected with an appropriate antibiotic (zeocin, blasticidin, hygromycin, or geneticin) to select a pool of cells with stable expression of the signaling receptor. The cells are loaded with beta-lactamase substrate and FACS is used to determine the percentage of cells expressing a measurable amount of activated signaling receptor (e.g., % blue cells) in the absence of an activating ligand.

Table 15 shows the results for several different cells expressing different cell signaling receptors. ADORA2a, PGE2R, PDR are in CRE-bla CHO-k1 cells (Invitrogen, Cat # K1129). M1, M3, M5 are in the NFAT-bla CHO-k1 cells (Invitrogen, Cat #K1078). M4 is in the Gqo5 NFAT-bla CHO-k1 cells (Invitrogen, Cat #K1220). CCKBR are in NFAT-bla HEK293T (Invitrogen, Cat #K1179). ERalpha are in the Griptite HEK UAS-bla. The ERα-UAS-bla GripTite™ 293 MSR cell line (Invitrogen, Cat#K1090) is similar to the ERalpha cells.

These results demonstrate that many if not most signaling receptors (e.g., GPCRs or nuclear receptors) can be expressed in an active state in the absence of an activating ligand. ERa is a nuclear receptor.

TABLE 15

| | % Constitutive via |
|---|---|
| Gene | FACS |
| ADORA2A | 5% |
| PDR | 2.30% |
| PE2R | 2.70% |
| M4 | 2.50% |
| CCKBR | 7.70% |
| M1 | 0.20% |
| M3 | 0.50% |
| M5 | 1.30% |
| Eralpha | 1.10% |

### Example 14

### Construction of an mG2A Expression Plasmid

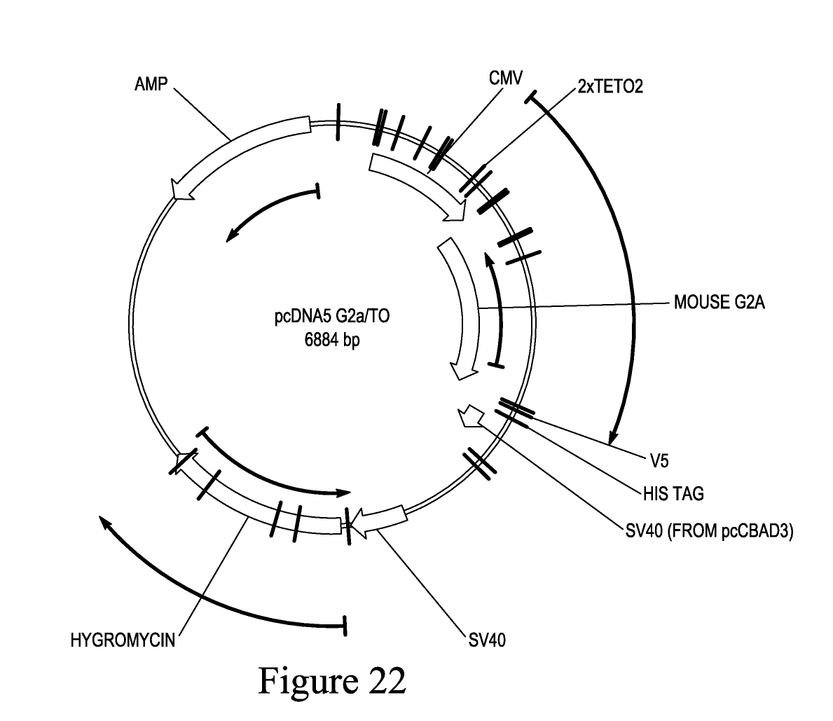

To create the T-REx™-mG2A-NFAT-bla Freestyle 293F cell line, mG2A was PCR amplified adding a BamH1 site to the 5′ end and a Not I site to the 3′ end. pcDNA5 TO G2A (See FIG. 3 and Example 1) was digested with BamH1 and Not I to remove the human G2A sequence. The large fragment was gel purified. The mouse G2A PCR fragments were digested with BamHI and NotI and purified. The mouse G2A fragment was ligated into the pcDNA5 TO vector and colonies were screen via PCR. Selected colonies were miniprepped and sequenced. The resulting plasmid was designated “pcDNA5 TO G2A (mouse)” (SEQ ID NO:14). This expression plasmid also contains a hygromycin antibiotic resistance gene. A map of this plasmid is shown in FIG. 22. The Tet Repressor plasmid is shown in FIG. 4. The coding region for mG2a is SEQ ID NO:15.

### Example 15

### Transient Transfections of an mG2A Expression Plasmid

In order to test whether or not mG2A showed the same or similar constitutive activity and deleterious effect on cell health upon overexpression in the NFAT-bla Freestyle 293F cell lines as human G2A, the mG2A vector was transiently transfected into NFAT-bla Freestyle 293F cells.

The NFAT-bla Freestyle 293F cells were transiently transfected with the mG2A plasmid for 48 hours. Lipofectamine 2000 (Invitrogen) was used at a ratio of 0.4 ug of DNA per every uL of Lipofectamine 2000 following manufacturer's directions. After 48 hours the cells were induced with the indicated concentration of doxycycline for 24 hours. The NFAT-bla TR Freestyle HEK 293 exhibits increased constitutive beta-lactamase expression when induced with doxycycline.

In the absence of the tet repressor protein (TR), expression of the mG2A is directly driven from the CMV promoter. Under these conditions, cell death and constitutive activity were observed with the mouse G2A receptor similar to that observed for the human G2A receptor. When the same mouse G2A vector was instead transiently transfected into TR-NFAT-bla Freestyle 293F cells (expressing a tet repressor protein), cell death and constitutive activity were not observed. It is believed TR was bound to the TetO₂ elements preventing overexpression of mG2A.

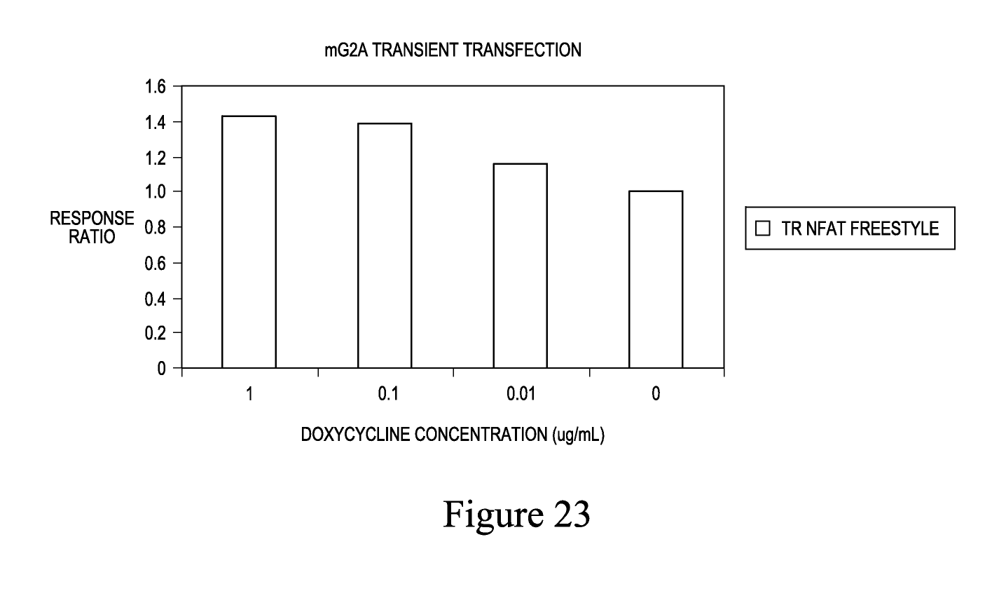

Transient transfection data for the TR NFAT-bla cell line is displayed in FIG. 23. mG2A under TREx control was transiently transfected into a pool of NFAT-bla Freestyle cells containing the tet repressor (TR). As increasing amounts of doxycycline are added and the expression of mG2A increases, the constitutive activity (blue/green ratio) increases.

### Example 16

### Stable Transfections of an mG2A Expression Plasmid and Clone Selection

### Stable Transfection

The CellSensor™ NFAT-bla FreeStyle™ 293F Cell Line (Catalog #K1097, Invitrogen) was transfected with pcDNA6/TRA to create a TREx™-NFAT-bla Freestyle 293F CellSensor™ Cell Line. This TREx™-NFAT-bla Freestyle 293F CellSensor™ Cell Line was transfected with the T-REx plasmid pcDNA 5/TO mG2A and selected with Hygromycin for approximately 2 weeks prior to sorting by flow cytometry. Lipofectamine 2000 (Invitrogen) was used at a ratio of 0.4 ug of DNA per every uL of Lipofectamine 2000 following manufacturer's directions.

### Sorting

Cells were trypsinized and loaded with Invitrogen's LiveBLAzer™-FRET B/G substrate (Catalog# K1030) for 2 hours prior to sorting. Stable T-REx mG2A NFAT-bla Freestyle 293F pools were sorted without tetracycline stimulation into green and turquoise pools. Green cells are ones in which the tet repressor (TR) is repressing expression of mG2A and thus is repressing constitutive activity. Turquoise cells are ones in which the TR is partially repressing expression of mG2A leading to low levels of constitutive activity. Blue cells are ones in which TR is not repressing mG2A activity which could be due to inactivity of TR in those cells. Data is shown in Table 16.

In addition, clones were obtained from the T-REx™ mG2A NFAT-bla Freestyle 293F turquoise pool utilizing FACS to distribute single-cells from the turquoise population.

TABLE 16

| | | % | Green 535/40-A | Blue 460/50-A |
|---|---|---|---|---|
| Population | #Events | Parent | Geometric Mean | Geometric Mean |
| [symbol] All Events | 14,933 | #### | #### | #### |
| [symbol] CELLS | 10,488 | 70.2 | #### | 546 |
| [symbol] GREEN | 3,582 | 34.2 | 8,893 | 148 |
| [symbol] BLUE | 219 | 2.1 | 1,874 | 9,734 |
| [symbol] TURQ | 5,916 | 56.4 | 8,760 | 1,073 |

### Induction Optimization

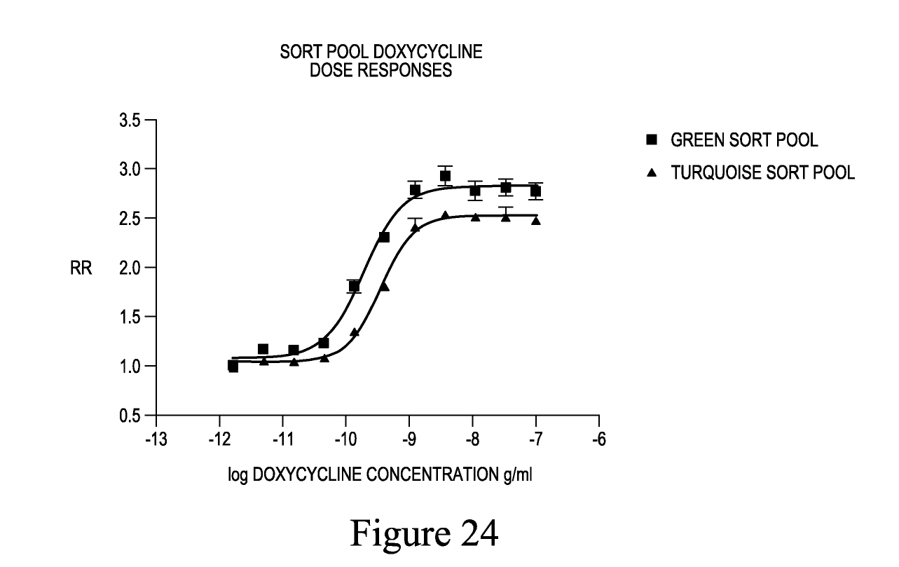

Since mG2A has no known agonists, doxycycline was used as an inducer of the assay. A 16 hour induction time was utilized for induction as this led to constitutive activity but not excessive cell death. Doxycycline dose response curves were obtained for both the green and the turquoise sorted pools. Data and concentrations of doxycycline are shown in FIG. 24.

### Clone Selection

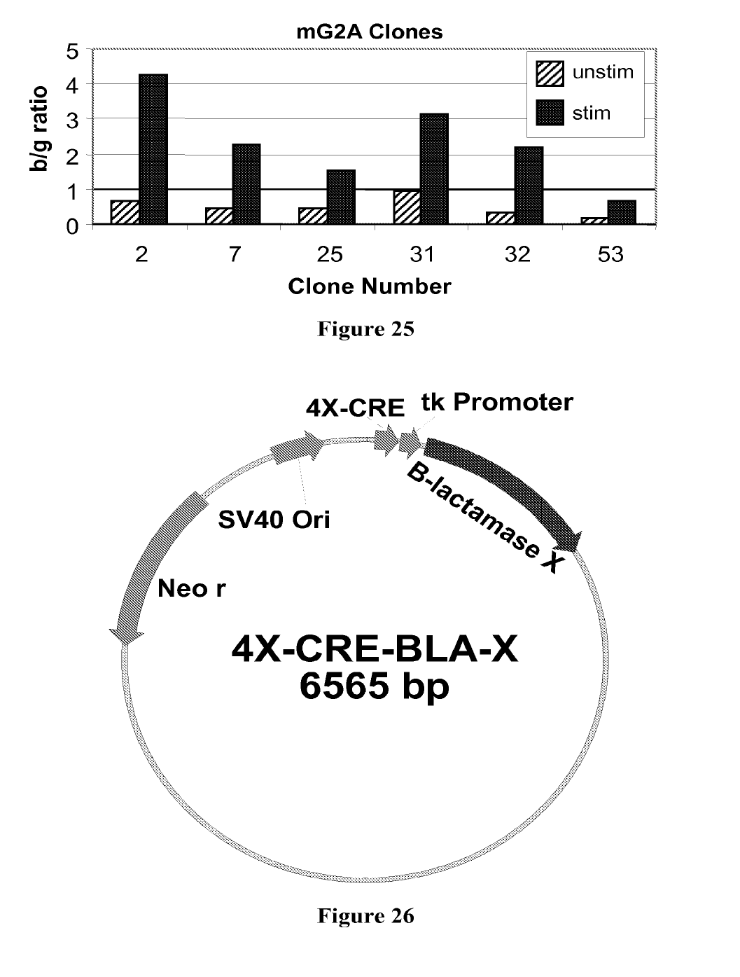

Clones obtained from the turquoise sort were screened for a response to doxycycline. Of the 64 clones tested, at least six clones showing desirable uninduced to induced ratios were selected for further testing. FIG. 25 shows blue/green ratios of six clones selected from the initial round of sorting. Clones were left in the uninduced state (unstim) or were induced (stim) for 16 hours with 100 ng/mL of doxycycline.

### RT-PCR Verification

In order to confirm that mouse G2A induction was causing the constitutive beta-lactamase activity seen in these clones, RT-PCR was carried out. RNA was harvested from both doxycycline stimulated (18 hours with 1 ng/mL) and unstimulated cells. Mouse G2A expression was observed in all three clones both before and after stimulation, although expression increased upon doxycycline treatment for some clones. As expected, no mouse G2A expression was observed in the TR parental cell line.

### RNAi Verification

Invitrogen's Stealth siRNA was used to confirm that the observed increase in beta-lactamase blue:green ratios was due to mG2A expression. Cells were transfected with siRNA for 48 hours and induced for 16 hours with either 0 ng/mL doxycycline or 1 ng/mL doxycycline prior to loading cells with the LiveBLAzer FRET-B/G substrate (Invitrogen) for 2 hours. The MedGC is a negative control siRNA made up of a random medium GC rich sequence. The BLA is a positive control consisting of siRNA directed towards beta-lactamase. The siRNA #1 is 25 bp, blunt ended, and double stranded. The sequence for siRNA #1 is “upper stand” 5′ to 3′ UUC AAA GGC ACA CAC GGC AUC CAU G (SEQ ID NO:12) and “lower stand” 5′ to 3′ CAU GGA UGC CGU GUG UGC CUU UGA A (SEQ ID NO:13). Data is shown for siRNA #1 directed towards mG2A.

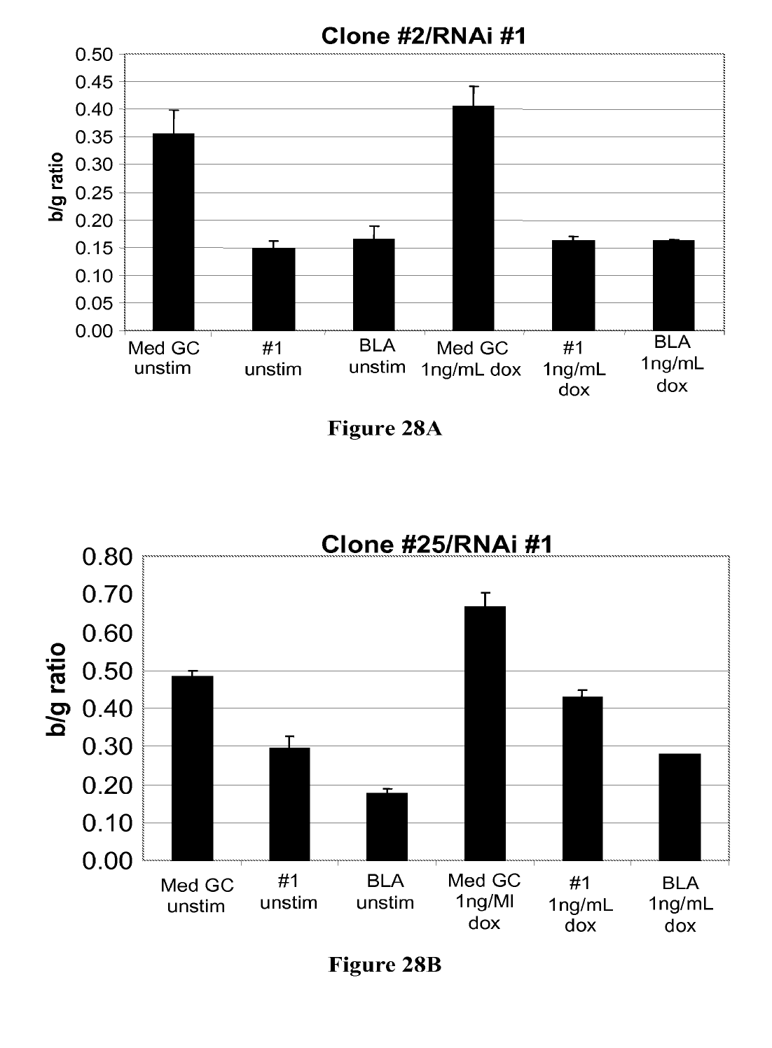

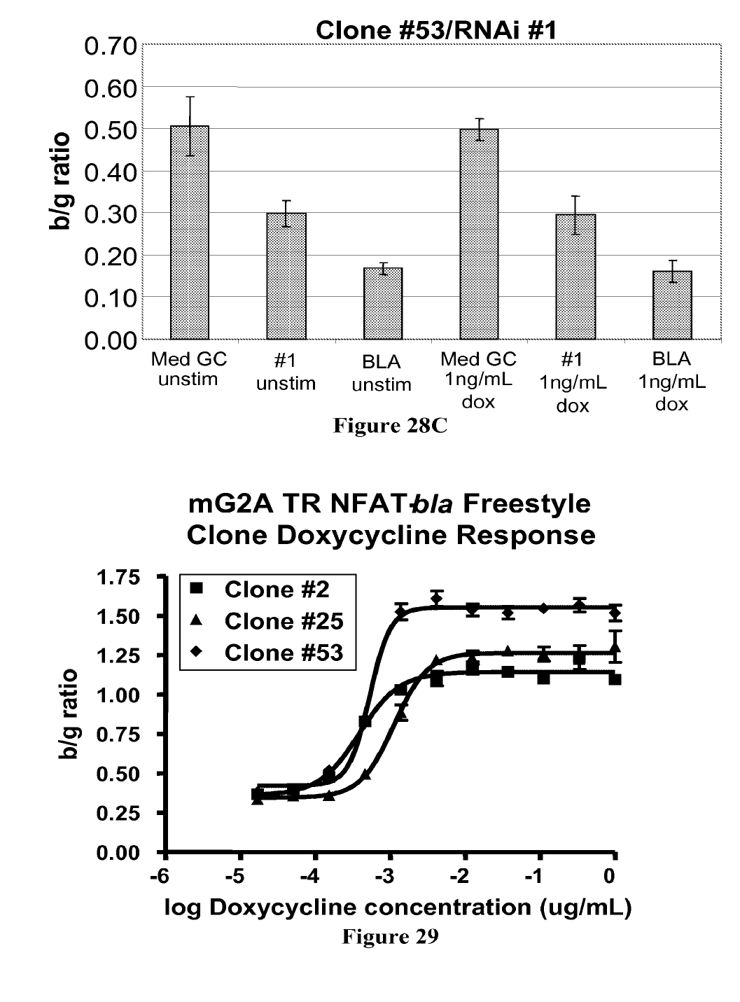

In all three clones tested, siRNAs directed against either mouse G2A or beta-lactamase strongly knocked down both the doxycycline-induced and the beta-lactamase signals relative to levels seen with the negative control MedGC siRNA. This confirms that the signaling seen in these cells is indeed mouse G2A dependent. RNAi data is displayed in FIGS. 28A-C.

An exemplary assay protocol that can be utilized with these cell lines is provided in Example 12.

### Example 17

### Construction of an hGPR23 Expression Plasmid

hGPR23 expression plasmids were constructed using the Invitrogen Gateway® technology. contains an hGPR23 open reading frame. Using standard Gateway® techniques the hGPR3 open reading frame from the Ultimate™ ORF IOH28360 clone (Invitrogen) (which matches GenBank Accession No. NM_{—}005296.1) was cloned into both the pLenti4/V5-DEST™ Gateway® Vector (catalog# V498-10, Invitrogen) and the pLenti6/V5-DEST™ Gateway® Vector (catalog# V496-10, Invitrogen).

The hGPR23 expression plasmid from pLenti4/V5-DEST™ contains a blasticidin resistance marker. This plasmid was used for transfections of cells containing an NFAT-bla expression construct.

The hGPR23 expression plasmid from pLenti6/V5-DEST™ contains a zeocin resistance marker. This plasmid was used for transfections of cells containing a CRE-bla expression construct.

### Example 18

### Transient Transfections of a GPR23 Expression Plasmid

Lysophosphatidic acid (LPA; 1 or 2-acyl-sn-glycero-3-phosphate) stimulation of GPR23 has been shown to stimulate adenylyl cyclase (Gₛ pathway) and intracellular Ca²⁺ mobilization (G_{q} pathway), e.g., see Lee et al., JBC papers in press, Dec. 13, 2006 as Manuscript M610826200.

To begin development of a GPR23 assay in GeneBLAzer® CellSensor™ cell lines (Invitrogen), GPR23 was first tested in a transient transfection assay in the NFAT-bla HEK (Catalog# K1179, Invitrogen), the CRE-bla HEK (Catalog# K1112, Invitrogen), the NFAT-bla CHO-K1 (Catalog# K1078, Invitrogen), the CRE-bla CHO-K1 (Catalog# K1129, Invitrogen), the NFAT-bla Jurkat CellSensor™ cell lines (Catalog# K1077, Invitrogen) and the CRE-bla Jurkat CellSensor™ cell lines (Catalog# K1134, Invitrogen). The hGPR23 expression plasmid was transfected into the cell lines. Lipofectamine 2000 (Invitrogen) was used at a ratio of 0.4 ug of DNA per every uL of Lipofectamine 2000 following manufacturer's directions. After 48 hours the cells were assayed for a response to 10 μM LPA.

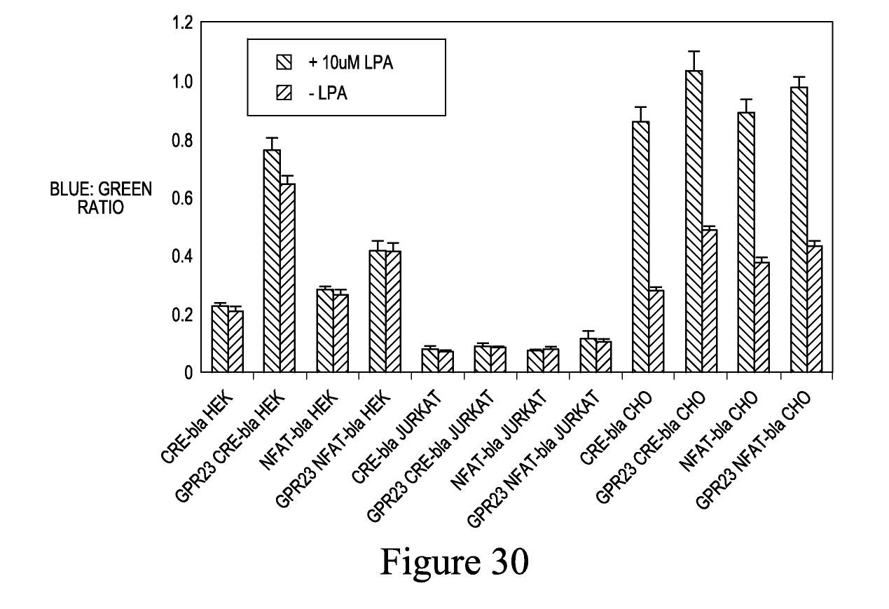

The NFAT and CRE CHO-K1 cell lines gave a positive response to LPA in the absence of transfected GPR23. This response may be due to endogenous expression of LPA receptors in this cell line. The Jurkat cells showed no endogenous response to LPA, and no response to LPA in GPR23 transfected cells. The HEK cells showed no endogenous response to LPA and only a small response to LPA in the GPR23 transfected CRE-bla HEK cell line. The HEK cells also had an increase in the background activity of the CRE reporter in the GPR23 transfected cells that was likely due to constitutive activity of the over expressed GPR23 receptor Results are shown in FIG. 30.

Assay construction continued in the NFAT-bla and CRE-bla, HEK and Jurkat CellSensor™ cell lines. These cell lines were taken to the point of stably selected pools at which point they were tested for a response to LPA. Only the stably transfected GPR23-CRE-bla HEK pool gave a response to LPA. The response was <1.5 fold but reproducible.

### Example 19

### Expression of Human GPR23 in the CellSensor™ CRE-bla CHO-K1 Cell Line

An attempt was made to develop a GPR23 assay by stably expressing a human GPR23 (hGPR23) in a CRE-bla CHO-K1 cell line. The CRE-bla CHO-K1 cell line has a non-GPR23 specific endogenous response to LPA (see Example 18), but by adding the human GPR23 to the cells it could have been possible to increase the LPA responsiveness of these cells or create a constitutively active cell line that could be used to screen for inverse agonists of the hGPR23 receptor. The hGPR23 expression plasmid was transfected into the CRE-bla CHO-K1 cell line. Lipofectamine 2000 (Invitrogen) was used at a ratio of 0.4 ug of DNA per every uL of Lipofectamine 2000 following manufacturer's directions.

A stable pool was selected using Zeocin. LPA dose response on the hGPR23-CRE-bla CHO-K1 selected pool and CRE-bla CHO-K1 cell line was performed. The cells were plated at 50,000 cells per well in a 96 well assay plate in OptiMEM+0.5% FBS and placed at 37° C. 5% CO₂ overnight. The cells were then stimulated with a four fold dilution series of LPA in DMEM+0.1% BSA for 5 hrs. and loaded with substrate for 2 hrs.

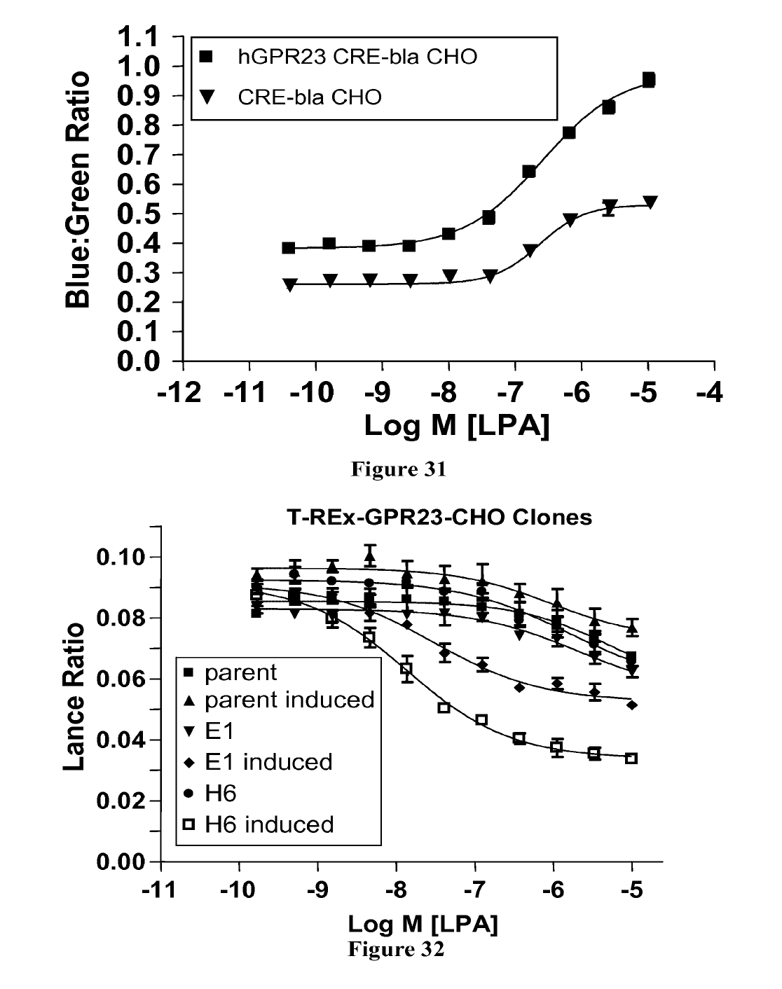

The hGPR23 stable pool gave a similar response to LPA as the CRE-bla CHO-K1 cell line with similar EC₅₀ values and maximum response ratios, however once again the GPR23 expressing cells showed some constitutive activity as indicated by the larger blue:green ratios. Results are shown in FIG. 31 with a resulting EC₅₀ to LPA of 258 nM for the hGPR23CRE-bla CHO cells and of 239 nM for the CRE-bla CHO cells.

In an effort to isolate an hGPR23 specific responding clone, the hGPR23-CRE-bla CHO-K1 selected pool was blind sorted for clones which would then be tested for their level of hGPR23 expression by bDNA (branched DNA) analysis and responsiveness to LPA. In total, eight plates of hGPR23-CRE-bla CHO-K1 clones were collected, and one plate of CRE-bla CHO-K1 clones was collected as a control. Once the clones had expanded, they were screened for a response to LPA.

All of the plates were analyzed to compare hGPR23 expression levels with response ratio and un-stimulated blue:green ratio. The results were obtained by comparing the hGPR23-CRE-bla CHO clone plates to the CRE-bla CHO clone control plate. The hGPR23-CRE-bla CHO and the CRE-bla CHO clones gave a similar range of responsiveness to LPA. The hGPR23-CRE-bla CHO clones showed no correlation between the level of hGPR23 expression as determined by bDNA analysis and the overall LPA responsiveness of the cells or the background CRE-bla activity as determined by the un-stimulated blue:green ratio.

### Example 20

### T-REx™-GPR23-CRE-bla-CHO-K1 Cellular Assay Development

A GPR23 coding region was placed into a vector under control of a tetracycline inducible promoter with zeocin resistance using a T-Rex™ kit (Invitrogen). Cells were also transfected with a vector containing the tet repressor and a blasticidin resistance coding region. Antibiotic selection was carried out and clones were obtained. Several inducible T-REx™-GPR23-CHO-K1 clones were obtained that showed a cAMP response to LPA stimulation. These cell lines were used as starting material to construct an assay product.

### T-REx™-GPR23-CHO-K1 Clones

Two inducible T-REx™-GPR23-CHO-K1 clones and a parental control (T-REx™-CHO-K1) were tested using the Perkin Elmer LANCE cAMP assay. The cells were serum starved and induced in growth medium +/−1 μg/ml tetracycline +100 ng/ml pertussis toxin without FBS for 24 hrs prior to the assay. The cells were then harvested using versine and assayed for LPA responsiveness using the Perkin Elmer Lance assay according to the manufacture's instructions.

The parent cell line gave a slight response to LPA in both the induced and un-induced state. This level of response is not unexpected and is likely due to the endogenous expression of LPA receptors in CHO-K1 cells. The level of the response in the parent cell line did not change with incubation of the cells with tetracycline. The T-REx-GPR23-CHO-K1 clones E1 and H6 both showed an increased responsiveness to LPA and a shift in the potency of LPA in the presence of tetracycline as compared to the absence of tetracycline. The results are shown in FIG. 32.

FIG. 32 shows the parent T-REx-CHO-K1 cell line that does not contain GPR23 gave no significant change in the responsiveness to LPA in the induced (▴) or un-induced (▪) state. The T-REx-GPR23-CHO-K1 E1 and H6 clones showed an increased responsiveness to LPA, and a shift in the EC50 value of LPA in the induced (♦,□) verses the un-induced (▾,) state. The LPA EC₅₀ results were: parent=5 μM; parent induced=915 nM; E1 clone=2 μM; E1 clone induced=30.3 nM; H6 clone=1.5 μM; and H6 induced=13.8 nM.

### Transfection and Selection of a T-REx™-GPR23-CRE-bla-CHO-K1 Stable Pool

The T-REx™-GPR23-CHO-K1 clones and the parental cell line were transfected with a CRE-bla reporter vector (p4X-CRE-BLA-X; SEQ ID NO:16; FIG. 26). A stable pool was then selected using 500 μg/ml Geneticin.

### Fluorescence Activated Cell Sorting of Forskolin Responding T-REx™-GPR23-CRE-bla-CHO-K1 Clones

The transfected and antibiotic selected T-REx™-CRE-bla-CHO-K1 and T-REx™-GPR23-CRE-bla-CHO-K1 clones were sorted for their responsiveness to forskolin. Forskolin activates adenylyl cyclase and results in increased intracellular cAMP concentrations which in turn lead to increased production of beta-lactamase from the CRE response element.

Forskolin response of the T-REx™-CRE-bla-CHO-K1 and T-REx™-GPR23-CRE-bla-CHO-K1 pools were determine using FACS. The T-REx™-CRE-bla-CHO-K1 selected pool had about a 10.7% increase in the percentage of cells in the “Blue” (responding) gate in the stimulated verses the un-stimulated populations. The T-REx™-GPR23-CRE-bla-CHO-K1 H6 and E1 had about a 21.1% and about a 20% increase in the “Blue” gate in the stimulated verses the un-stimulated populations. In addition, there was an increase in the constitutively active cells in the human GPR23 containing cells as compared to the parental as indicated by the un-stimulated blue levels. The responsive clones were collected at one cell per well in 96 well tissue culture plates then expanded and tested for there responsiveness to LPA.

### T-REx™-GPR23-CRE-bla-CHO-K1 Clone Selection

The sorted clones were allowed to grow in the 96 well plates until near confluence was reached. These clones were particularly slow growing after the sort. It took the clones over a month to grow to near confluence. This usually takes only two or three weeks for other transfected CHO-K1 cells. However, the clones showed more typical growth characteristics after they were passed out of the initial sorting wells.

The clones were then tested for a response to 10 μM LPA in the induced and un-induced state to select the best responding clones with which to continue. During this clone selection phase, individual clones from the 96 well plates were split into two wells on two plates to allow for screening of a functional response, and into one well on a separate plate for continued growth and expansion. Both of the assay plates were serum starved overnight (˜16 hrs) in serum free media containing 100 ng/ml pertussis toxin prior to assaying the clones for an LPA response. One of the plates was induced for GPR23 expression with 1 μg/ml tetracycline (˜16 hrs) and the other was left un-induced. The clones were then stimulated with 10 μM LPA for 5 hrs and loaded with LiveBLAzer™-FRET B/G substrate (2 μM) containing solution D (Invitrogen, Catalog# K1156) for 2 hrs.

The initial screen of the clones showed a mixed population of LPA responsive clones in both the T-REx™-CRE-bla-CHO-K1 and T-REx™-GPR23-CRE-bla-CHO-K1 plates. All of the clones were somewhat responsive to LPA. Since the CHO-K1 background cell line has been shown to be responsive to LPA this was not unexpected. There were no T-REx™-GPR23-CRE-bla-CHO-K1 clones that out-responded the T-REx™-CRE-bla-CHO-K1 clones. No clones were chosen for further development based on there responsiveness to LPA. One overall trend noticed is that the GPR23 containing clones consistently gave increased blue:green ratios in the induced verses un-induced state. This trend was not seen in the T-REx™-CRE-bla-CHO-K1 clones. This increased signal may be due to constitutive activity of the GPR23 receptor. Six of the T-REx™-GPR23-CRE-bla-CHO-K1 clones that showed the greatest inducible GPR23 constitutive activity were chosen for further development, e.g., as an inverse agonist assay.

Six clones selected for further testing were expanded and retested for their inducible GPR23 specific activity. The clones were plated at 25,000 cells per well in a 96 well black walled clear bottom tissue culture assay plate. The plates were than placed at 37° C. 5% CO₂ for 24 hrs to allow the cells to attach to the assay plate. The complete media was then removed from the plate and replaced with assay media (DMEM+0.1% BSA) containing 100 ng/ml pertussis toxin with or without 1 μg/ml tetracycline. The plates were placed at 37° C. 5% CO₂ for 16 hrs to allow the induced expression of the GPR23 receptor. The cells were then loaded with LiveBLAzer™-FRET B/G substrate (2 μM) containing solution D for 2 hrs.

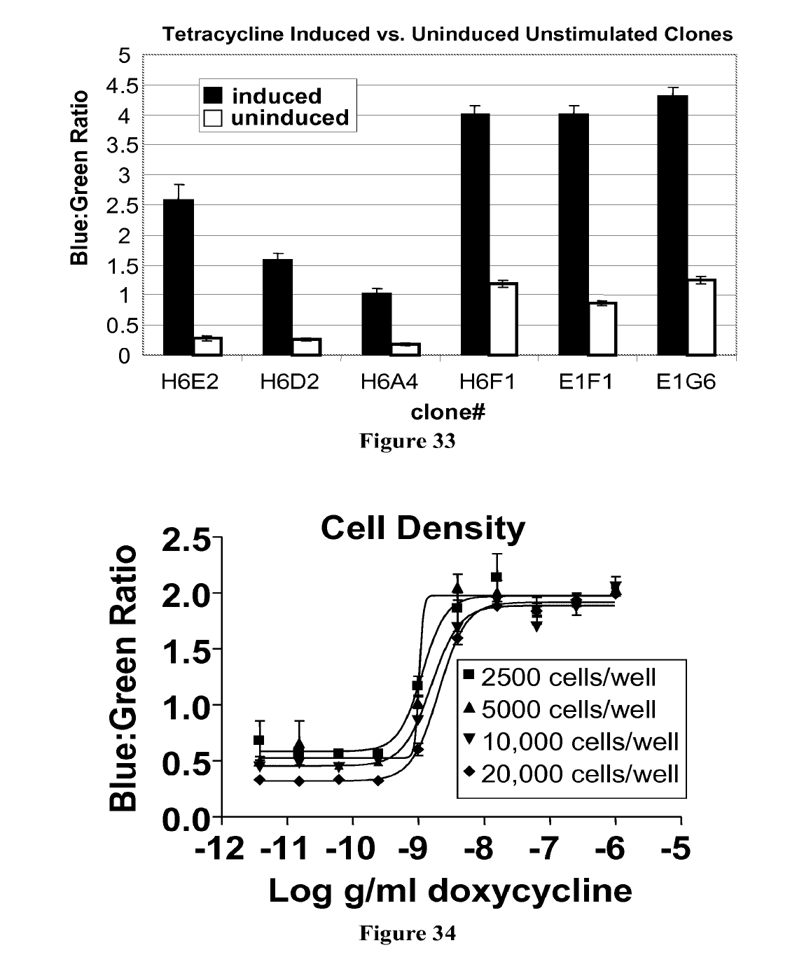

All six of the clones gave an inducible response to the tetracycline induction of GPR23 expression. Clone H6-E2 gave the greatest inducible response (about 9.2 fold) and was chosen as a clone for an inverse agonist assay for GPR23 (see FIG. 33).

### Example 21

### T-REx™-GPR23-CRE-bla-CHO-K1 Clone H6-E2 Cell Line Validation

### Cell Density Optimization

To determine the optimal cell density for this assay the cells were plated at 2,500, 5,000, 10,000, and 20,000 cells per well in a 384 well black walled clear bottom tissue culture assay plate in complete media (DMEM+10% FBS). The plates were than placed at 37° C. 5% CO₂ for 24 hrs to allow the cells to attach to the assay plate. The complete media was then removed from the plate and replaced with assay media (DMEM+0.1% BSA) containing 100 ng/ml pertussis toxin and a four fold dilution series of doxycycline starting at 1 μg/ml. The plates were placed at 37° C./5% CO₂ for 16 hrs to allow the induced expression of the GPR23 receptor. The cells were then loaded with LiveBLAzer™-FRET B/G substrate (2 μM) containing solution D for 2 hrs.

The assay performed the best plating 20,000 cells per well with a maximum response ratio of 5.7 fold and a Z′ value of 0.8. The assay could also be run at 10,000 or 5,000 cells per well with only a small effect on the assay window. The EC₅₀ values for doxycycline were 1.3 ng/ml, 1.0 ng/ml, 1.6 ng/ml and 2.0 ng/ml for 2,500, 5,000, 10,000, and 20,000 cells/well, respectively. Results are shown in FIG. 34.

### DMSO Tolerance

The cells were plated at 20,000 cells per well in a 384 well black walled clear bottom tissue culture assay plate in complete media (DMEM+10% FBS). The plates were then placed at 37° C. 5% CO₂ for 6 hrs to allow the cells to attach to the assay plate. The complete media was then removed from the plate and replaced with assay media (DMEM+0.1% BSA) containing 100 ng/ml pertussis toxin, 0%, 0.25%, 0.5%, or 1.0% DMSO, and a three fold dilution series of doxycycline starting at 1 μg/ml. The plates were placed at 37° C./5% CO₂ for 16 hrs to allow the induced expression of the GPR23 receptor. The cells were then loaded with LiveBLAzer™-FRET B/G substrate (2 μM) containing solution D for 2 hrs.

The DMSO concentration in the assay had no significant affect on the induction of GPR23 expression or the receptor's constitutive activity up to 1.0% DMSO. The EC₅₀ values for doxycycline were 3.3 ng/ml, 4.0 ng/ml, 4.0 ng/ml and 5.1 ng/ml for 0%, 0.25%, 0.5%, and 1.0% DMSO, respectively.

### Induction Time

The cells were plated at 20,000 cells per well in a 384 well black walled clear bottom tissue culture assay plate in complete media (DMEM+10% FBS). The plates were than placed at 37° C. 5% CO₂ for 6 hrs to allow the cells to attach to the assay plate. The complete media was then removed from the plate and replaced with assay media (DMEM+0.1% BSA) containing 100 ng/ml pertussis toxin, 0.5% DMSO, and a three fold dilution series of doxycycline starting at 1 μg/ml. The plates were placed at 37° C. 5% CO₂ for 16, 20 or 24 hrs to allow the induced expression of the GPR23 receptor. The cells were then loaded with LiveBLAzer™-FRET B/G substrate (2 μM) containing solution D for 2 hrs.

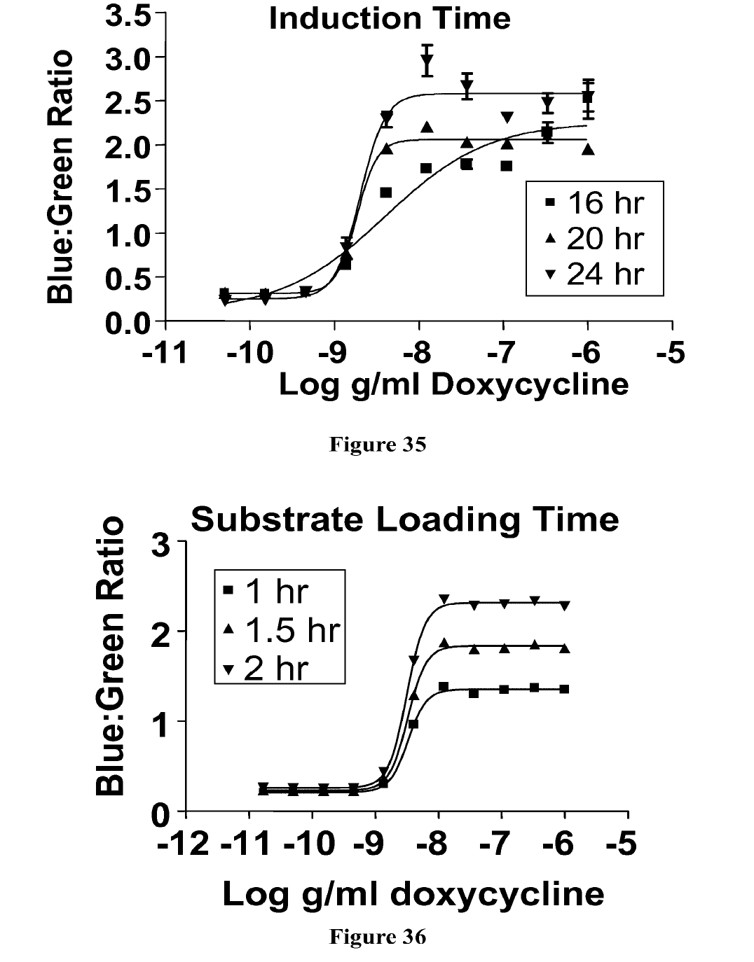

The widest assay window was achieved with a 24 hr induction time. The EC₅₀ values for doxycycline were 4.0 ng/ml, 1.9 ng/ml and 2.0 ng/ml for 16, 20 and 24 hours respectively. Results are shown in FIG. 35.

### GeneBLAzer® Substrate Loading Time

The cells were plated 20,000 cells per well in a 384 well black walled clear bottom tissue culture assay plate in complete media (DMEM+10% FBS). The plates were than placed at 37° C. 5% CO₂ for 6 hrs to allow the cells to attach to the assay plate. The complete media was then removed from the plate and replaced with assay media (DMEM+0.1% BSA) containing 100 ng/ml pertussis toxin, 0.5% DMSO, and a three fold dilution series of doxycycline starting at 1 μg/ml. The plates were placed at 37° C. 5% CO₂ for 16 hrs to allow the induced expression of the GPR23 receptor. The cells were then loaded with LiveBLAzer™-FRET B/G substrate (2 μM) containing solution D for 1, 1.5 or 2 hrs. The widest assay window was achieved with a 2 hr substrate loading time. Results are shown in FIG. 36.

### Assay Reproducibility

To analyze the reproducibility of the T-REx™-GPR23-CRE-bla-CHO-K1 cell line in an assay, an assay was run on three separate days and the results of the assays were compared for their consistency. The first (day 1; cell culture passage 3) and second (day 14; culture passage 7) replicates of the assay gave very similar results. The third replicate of the assay (day 19; culture passage 8) had about a 30% decrease in the maximum response of the cells to doxycycline. This drop in assay response was between the seventh and eighth passage of the culture. The assay was tested again on the ninth (day 20) and tenth (day 25) and twelfth (32) passage of the culture and the results confirmed the drop in the responsiveness of the cell line.

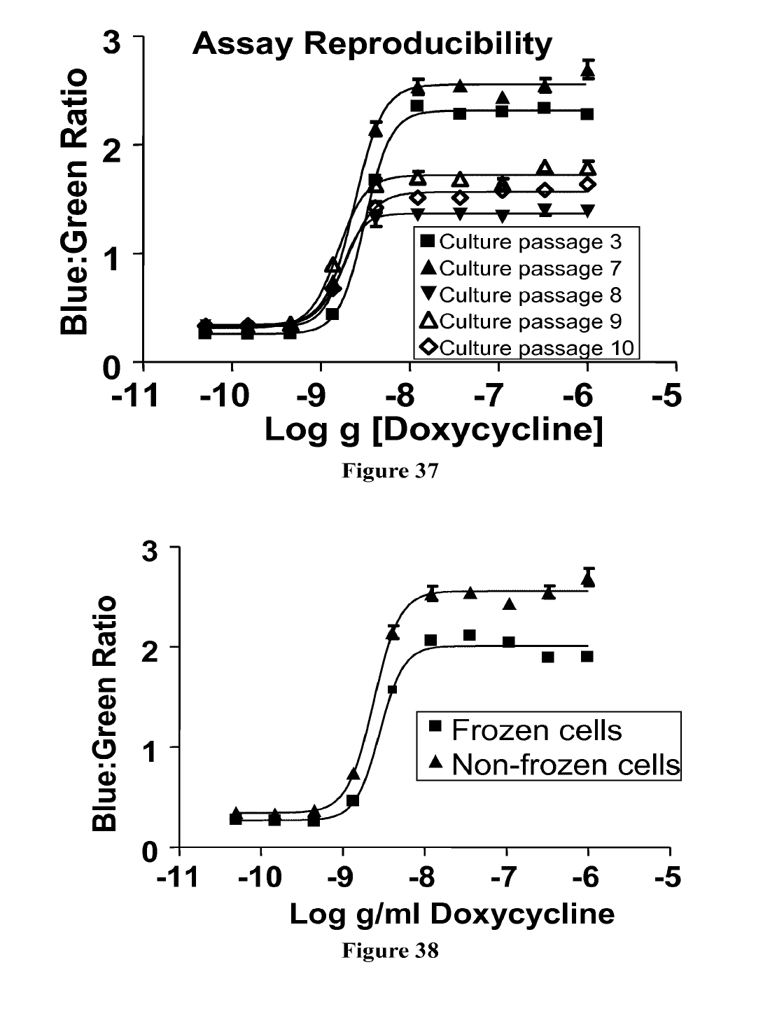

The cells were plated at 20,000 cells per well in a 384 well black walled clear bottom tissue culture assay plate in complete media (DMEM+10% FBS). The experiments performed on day 25 and day 32 were performed in Poly-D-Lysine coated plates. The plates were then placed at 37° C. 5% CO₂ for 6 hrs to allow the cells to attach to the assay plate. The complete media was then removed from the plate and replaced with assay media (DMEM+0.1% BSA) containing 100 ng/ml pertussis toxin, 0.5% DMSO, and a three fold dilution series of doxycycline starting at 1 μg/ml. The plates were placed at 37° C. 5% CO₂ for 16 hrs to allow the induced expression of the GPR23 receptor. The cells were then loaded with LiveBLAzer™-FRET B/G substrate (2 μM) containing solution D for 2 hrs. Results are shown in FIG. 37.

The assay had a drop in the maximum response to doxycycline between the second and third replicate of the assay. This drop in response was stable over the next two runs of the assay. The experiments performed on day 25 and day 32 were in Poly-D-Lysine coated plates to aid in cell adherence in the serum free media. The Z′ values are significantly improved if the coated plates are used (compare the experiments from day 19 or day 20 (non-coated) and day 25 and day 32 (coated)). Therefore, Poly-D-Lysine plates can be used to help improve the assay performance, e.g., in HTS.

### Frozen Cell Assay

Cells taken from LN₂ storage were thawed and plated at 20,000 cells per well in a 384 well black walled clear bottom tissue culture assay plate in complete media (DMEM+10% FBS). The plates were than placed at 37° C. 5% CO₂ for 6 hrs to allow the cells to attach to the assay plate. The complete media was then removed from the plate and replaced with assay media (DMEM+0.1% BSA) containing 100 ng/ml pertussis toxin, 0.5% DMSO, and a three fold dilution series of doxycycline starting at 1 μg/ml. The plates were placed at 37° C. 5% CO₂ for 16 hrs to allow the induced expression of the GPR23 receptor. The cells were then loaded with LiveBLAzer™-FRET B/G substrate (2 μM) containing solution D for 2 hrs. The assay was run side by side with non-frozen cells. Non-frozen cells were cells taken directly from culture that have been passed at least 3 times since being frozen before being assayed. Results are shown in FIG. 38.

There was no significant change in the assay window or the Z′ values of the assay when it was run using recently thawed cells.

### LPA Responsiveness of the T-REx™-GPR23 CRE-bla-CHO-K1 Clone H6-E2

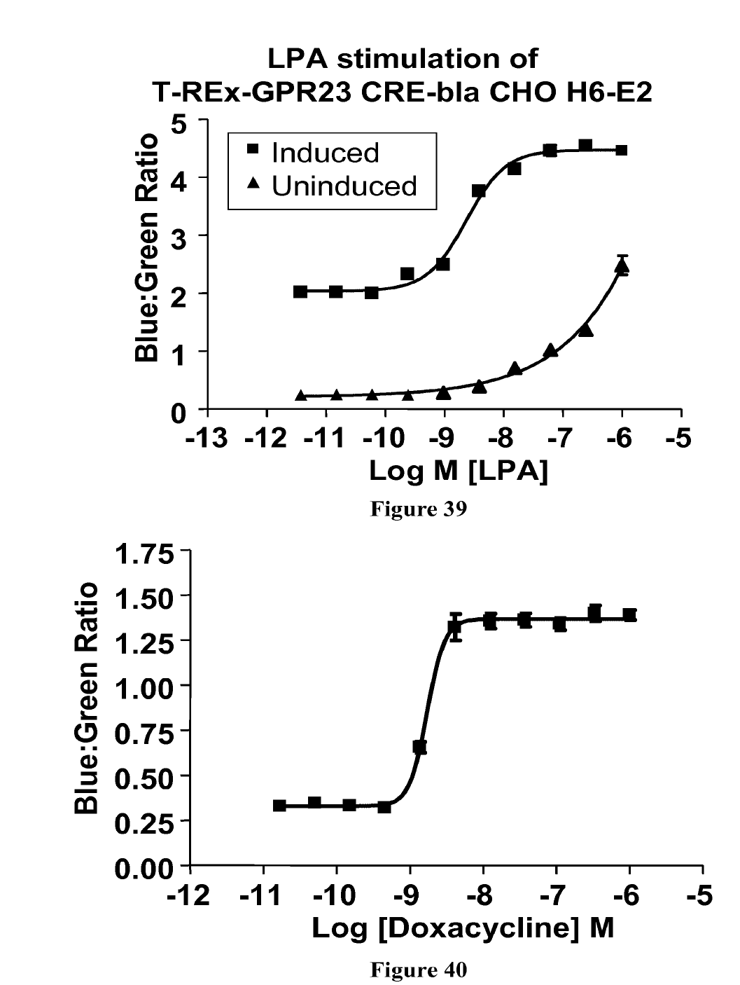

The cells were plated at 20,000 cells per well in a 384 well black walled clear bottom tissue culture assay plate in complete media (DMEM+10% FBS). The plates were then placed at 37° C. 5% CO₂ for 6 hrs to allow the cells to attach to the assay plate. The complete media was then removed from the plate and replaced with assay media (DMEM+0.1% BSA) containing 100 ng/ml pertussis toxin and 0.5% DMSO, with or without 10 μg/ml doxycycline. The plates were placed at 37° C. 5% CO₂ for 16 hrs to allow the induced expression of the GPR23 receptor. The cells were then stimulated for five hours with a four fold dilution series of LPA starting at 1 μM. The cells were then loaded with LiveBLAzer™-FRET B/G substrate (2 μM) containing solution D for 2 hrs. Results are shown in FIG. 39.

The induced T-REx™-GPR23-CRE-bla-CHO-K1 Clone H6-E2 cells showed a shifted EC₅₀ of LPA to 2.3 nM from the 628 μM of the un-induced cells. The response of the cells to LPA decreases from 9 fold in the un-induced cells to 2.3 fold in the induced cells due to the constitutive activity of the receptor.

Induced T-REx™-GPR23-CRE-bla-CHO-K1 cells can be utilized for various assays including screening or analyzing agonists and/or inverse agonists.

### Example 22

### An Exemplary Assay Protocol

The exemplary methods described in this example are set out in a format which is conducive for insertion into product literature of the invention.

The exemplary assay protocol below uses T-REx-GPR23-CRE-bla CHO cells as an exemplary cell line. However, very similar and even the same protocol can be utilized with other cell lines. One skilled in the art can determine how to use the exemplary assay protocol herein with other cell lines.

The following are sections for an exemplary assay protocol. In some embodiments, these sections can replace the sections as set forth in Example 12.

### 2.0 MATERIALS SUPPLIED

### Cell Line Name: T-REx-GPR23-CRE-bla CHO

Description: T-REx-GPR23-CRE-bla CHO contains the GPR23 receptor and the CRE-bla reporter stably integrated into the CHO-K1 cell line. The GeneBLAzer® CRE-bla reporter contains a beta-lactamase reporter gene under control of the CRE response element. The T-REx-GPR23-CRE-bla CHO cells have been shown to give a positive constitutive GPR23 specific activity when the cells are induced with doxycycline for GPR23 expression. This cell line can be used to screen for inverse agonists of the GPR23 receptor.

### Shipping Condition: Dry ice

Storage Condition: Liquid nitrogen. Immediately upon receipt, cells must be stored in liquid nitrogen or thawed immediately for use. Cells stored at −80° C. can quickly lose viability.
Quantity: ˜2,000,000 cells (2×10⁶ cells/ml)
Application: This cell line can be used to detect Inverse Agonists of GPR23.

### Growth Properties: Adherent

### Cell Phenotype: Epithelial

Selection Marker(s): Geneticin™ (500 μg/ml)

### Mycoplasma Testing: Negative

### BioSafety Level: 1

### 3.0 MATERIALS REQUIRED, BUT NOT SUPPLIED

TABLE 17

| Media/Reagents | Recommended | Part # |
|---|---|---|
| LiveBLAzer ™-FRET B/G Loading Kit | Invitrogen | K1030 |
| LiveBLAzer ™-FRET B/G Substrate | | Other |
| (CCF4-AM), 5 mg DMSO for Solution A | | sizes or |
| Solution B | | Loading |
| Solution C | | Kits |
| | | containing |
| | | CCF2-AM |
| | | are |
| | | available |
| Solution D | Invitrogen | K1156 |
| Cell Culture Freezing Medium | Invitrogen | 11101-011 |
| DMEM (high-glucose) | Invitrogen | 10569-010 |
| Phenol red-free DMEM | Invitrogen | 21063-029 |
| DMSO | Fluka | 41647 |
| Fetal bovine serum (FBS), dialyzed, tissue- | Invitrogen | 26400-036 |
| culture grade (DO NOT SUBSTITUTE!) | | |
| BSA | Various | — |
| Non-essential amino acids (NEAA) | Invitrogen | 11140-050 |
| Penicillin/Streptomycin (antibiotics) | Invitrogen | 15140-122 |
| Phosphate-buffered saline without calcium | Invitrogen | 14190-136 |
| and magnesium [PBS(−)] | | |
| HEPES (1 M, pH 7.3) | Invitrogen | 15630-080 |
| Doxycycline | MP Biomedical | 195044 |
| 0.05% Trypsin/EDTA | Invitrogen | 25300-054 |
| Geneticin (antibiotic) | Invitrogen | 10131-035 |
| Zeocin ™ (antibiotic) | Invitrogen | 46-0509 |
| Blasticidin (antibiotic) | Invitrogen | 46-1120 |
| | Recommended | |
| Consumables | Source | Part # |
| BioCoat Black-wall, clear-bottom, 384-well | BD | 35 4663 |
| plates | | |
| Compressed air | Various | — |

| Equipment | Recommended Source |
|---|---|
| Fluorescence plate reader with bottom-read | Various |
| capabilities | |
| Filters if required for plate reader | Chroma Technologies |
| (see Section 5.3.1) | |

### 4.0 CELL CULTURE CONDITIONS

### 4.1 Media Required

TABLE 18

| | Growth | Growth | | |
|---|---|---|---|---|
| | Medium | Medium | Assay | Freeze |
| Component | (−) | (+) | Medium | Medium |
| DMEM | 90% | 90% | — | — |
| Phenol red-free | — | — | 99.9% | — |
| DMEM | | | | |
| Dialyzed FBS | 10% | 10% | — | — |
| BSA | — | — | 0.1% | — |
| NEAA | 0.1 mM | 0.1 mM | — | — |
| HEPES (pH 7.3) | 25 mM | 25 mM | — | — |
| Penicillin (antibiotic) | 100 U/ml | 100 U/ml | — | — |
| Streptomycin | 100 μg/ml | 100 μg/ml | — | — |
| (antibiotic) | | | | |
| Geneticin (antibiotic) | — | 500 μg/ml | — | — |
| Zeocin ™ (antibiotic) | — | 250 μg/ml | — | — |
| Blasticiden | — | 10 μg/ml | — | — |
| Cell Culture Freezing | — | — | — | 100% |
| Medium | | | | |

Note:
Unless otherwise stated, have all media and solutions at least at room temperature (we recommend 37° C. for optimal performance) before adding them to the cells.

### 4.2 Growth Conditions

1. Thaw cells in Growth Medium (−) and grow in Growth Medium (+). Passage or feed cells at least twice a week and maintain them in a humidified 37° C./5% CO₂ incubator. Maintain cells at between 5% and 95% confluence. Cells should not be allowed to reach confluence.
2. Freeze cells at 2×10⁶ cells/ml in Freeze Medium.
3. For detailed cell growth and maintenance directions see Appendix A.

### 5.0 ASSAY PROCEDURE

The following instructions outline the recommended procedure for determining activity of compounds as modulators of GPR23 using LiveBLAzer™-FRET B/G Substrate as the readout. If alternative substrates are used (e.g., ToxBLAzer™ DualScreen or LyticBLAzer™ Loading kits), follow the loading protocol provided with the product.

### 5.1 Quick Assay Reference Guides (for a More Detailed Protocol, See Section 5.2).

TABLE 19
Inverse Agonist Assay Quick Reference Guide

| | Un-induced Wells | Induced Wells | Cell-free Wells | Test Compound Wells |
|---|---|---|---|---|
| Step 1 | 32 μl cells in | 32 μl cells in | 32 μl Growth | 32 μl cells in |
| Plate cells | Growth Medium | Growth Medium | Medium | Growth Medium |
| | (20,000 cells/well) | (20,000 cells/well) | (no cells) | (20,000 cells/well) |
| Step 2 | Incubate at 37° C. for | Incubate at 37° C. | Incubate at 37° C. | Incubate at 37° C. |
| Incubate cells | 6 hours | for 6 hours | for 6 hours | for 6 hours |
| Step 3 | Aspirate Growth | Aspirate Growth | Aspirate Growth | Aspirate Growth |
| Change medium | Medium and replace | Medium and | Medium and | Medium and |
| | with 32 μl Assay | replace with 32 μl | replace with 32 μl | replace with 32 μl |
| | Medium. | Assay Medium | Assay Medium | Assay Medium |
| Step 4 | 4 μl Assay Medium | 4 μl 10X | 4 μl 10X | 4 μl 10X |
| Induce GPR23 | with 1 μg/ml | doxycycline in | doxycycline in | doxycycline in |
| expression | pertussis toxin | Assay Medium | Assay Medium | Assay Medium |
| | | with 1 μg/ml | with 1 μg/ml | with 1 μg/ml |
| | | pertussis toxin | pertussis toxin | pertussis toxin |
| Step 5 | Incubate at 37° C. for | Incubate at 37° C. | Incubate at 37° C. | Incubate at 37° C. |
| Incubate cells | 16-24 hours | for 16-24 hours | for 16-24 hours | for 16-24 hours |
| Step 6 | 4 μl Assay Medium | 4 μl Assay | 4 μl Assay | 4 μl 10X Test |
| Add Inverse | with 5% DMSO | Medium with 5% | Medium with 5% | Compounds in 5% |
| Agonist or Test | | DMSO | DMSO | DMSO |
| Compounds | | | | |

| | |
|---|---|
| Step 5 | Incubate in a humidified 37° C./5% CO₂ incubator for 5 hours |
| Incubate cells | |
| Step 6 | 12 μl of 1 mM LiveBLAzer ™-FRET B/G (CCF4-AM) Substrate + 60 μl of |
| Prepare 6X | solution B + 30 μl of solution D, mix. Add 898 μl of Solution C, mix |
| Substrate Mix. | |
| Step 7 | 8 μl per well |
| Add Substrate | |
| Mixture | |
| Step 8 | 2 hours at room temperature in the dark |
| Incubate | |
| Substrate Mix. + | |
| cells | |
| Step 7 | See Section 5.3 |
| Detect activity | |
| Step 8 | See Section 6.0 |
| Analyze data | |

### 5.2 Detailed Assay Protocol

Plate layouts and experimental outlines will vary; in screening mode, we recommend using at least three wells for each control: Unstimulated Control, Stimulated Control, and Cell-free Control.

Note: Some solvents may affect assay performance. Assess the effects of solvent before screening. The cell stimulation described below is carried out in the presence of 0.5% DMSO to simulate the effect that a Test Compound solvent might have on the assay. If you use other solvents and/or solvent concentrations, change the following assay conditions and optimize appropriately.

### 5.2.1 Precautions

1. Work on a dust-free, clean surface. Always handle the 384-well, black-wall, clear-bottom assay plate by the sides; do not touch the clear bottom of the assay plate.
2. If pipetting manually, you may need to centrifuge the plate briefly at room temperature (for 1 minute at 14×g) after additions to ensure all assay components are on the bottom of the wells.

### 5.2.2 Plating Cells

### Day 1 (Day Before the Assay):

### (Morning)

1. Harvest cells and resuspend in Growth Medium (+) at a density of 6.25×10⁵ cells/ml.
2. Add 32 μl per well of the Growth Medium (+) to the Cell-free Control wells. Add 32 μl per well of the cell suspension to the Test Compound wells, the Unstimulated Control wells, and Stimulated Control wells.
3. Plate cells the morning of the day before the assay and place them in a humidified 37° C./5% CO₂ incubator for 6 hours to allow the cells to attach to the assay plate.

### (Afternoon)

4. Remove the Growth Medium from the wells of the plate and replace with 32 μl Assay Medium per well.
5. Prepare a stock solution of 1 μg/ml pertussis toxin in Assay Medium.
6. Prepare a 10× stock of doxycycline in Assay Medium with 1 μg/ml pertussis toxin.
7. Add 4 μl of the 10× stock of doxycycline to the Cell-free Control wells, Test Compound wells, and the Induced Control wells.
8. Add 4 μl of the 1 μg/ml pertussis toxin in Assay Medium to the Un-induced Control wells.
9. Incubate the assay plate in a humidified 37° C./5% CO₂ incubator for 16-24 hours to allow expression of GPR23.

### Day 2 (Day of Assay):

### 5.2.3 Inverse Agonist Assay Plate Setup

Note: This subsection provides directions for performing an Inverse Agonist assay.
1. Prepare a stock solution of 5% DMSO in Assay Medium.
2. Prepare a 10× stock of Test Compounds in Assay Medium with 5% DMSO.
3. Add 4 μl of the stock solution of 5% DMSO in Assay Medium to the Un-induced Control wells, Induced Control wells and Cell-free Control wells.
4. Add 4 μl of the 10× stock of Test Compounds to the Test Compound wells.
5. Incubate the Inverse Agonist assay plate in a humidified 37° C./5% CO₂ incubator for 5 hours.

### 5.2.4 Substrate Loading and Incubation

This protocol is designed for loading cells with LiveBLAzer™-FRET B/G Substrate Mixture (CCF4-AM) or CCF2-AM. If you use alternative substrates, follow the loading protocol provided with the substrate.

Prepare LiveBLAzer™-FRET B/G Substrate Mixture (CCF4-AM) or CCF2-AM Substrate Mixture and load cells in the absence of direct strong lighting. Turn off the light in the hood.
1. Prepare Solution A: 1 mM LiveBLAzer™-FRET B/G Substrate (CCF4-AM) in dry DMSO. Store the aliquots of the stock solution at −20° C. until use. The molecular weight of CCF2-AM is 1082 g/mol, and the molecular weight of the LiveBLAzer™-FRET B/G Substrate (CCF4-AM) is 1096 g/mol.
2. Prepare 6× Loading Solution:
   Add 12 μl of Solution A to 60 μl of Solution B and vortex.
   Add 30 μl of Solution D to the above solution and vortex.
   Add 898 μl Solution C to the above solution and vortex.
3. Remove assay plate from the humidified 37° C./5% CO₂ incubator.
4. Add 8 μl of the 6× Substrate Mixture to each well.
5. Cover the plate to protect it from light and evaporation.
6. Incubate at room temperature for 2 hours.

Note: Handle the plate gently and do not touch the bottom.

### 6.3 Representative Data

Dose response of T-REx-GPR23-CRE-bla CHO-K1 cells to Doxycycline. T-REx-GPR23-CRE-bla CHO-K1 cells were plated in Growth Media (+) and allowed to attach to the tissue culture plate for 6 hrs. They were then induced for GPR23 expression overnight with doxycycline for 16 hrs in Assay Media. Cells were then loaded with LiveBLAzer™-FRET B/G Substrate (CCF4-AM) for 2 hours. Fluorescence emission values at 460 nm and 530 nm were obtained using a standard florescence plate reader and the Blue/Green Emission Ratios were plotted against the indicated concentrations of doxycycline (FIG. 40).

TABLE 20

| EC₅₀ | 1.7 nM |
|---|---|
| EC₁₀₀ | 6.2 μM |
| Z′ at EC₁₀₀ | 0.58 |

1. A cell comprising a first nucleic acid, wherein the first nucleic acid comprises a regulatable promoter operatively linked to a G-protein-coupled receptor (GPCR) coding region and wherein the cell is capable of expressing the GPCR in an activated state.

2. The cell of claim 1, wherein the GPCR is selected from the group consisting of a class A GPCR; a class B GPCR; a class C GPCR; a Frizzled and Smoothened-related receptor; an adhesion family receptor; an adiponectin receptor; and a chemosensory receptor.

3. The cell of claim 1, wherein the cell is a stable cell.

4. The cell of claim 1, wherein the cell is capable of expressing an GPCR in an activated state in the absence of a ligand for the GPCR.

5. The cell of claim 1, wherein the regulatable promoter is selected from the group consisting of a tetracycline inducible promoter, heat shock inducible promoter, a heavy a metal ion inducible promoter, or a nuclear hormone receptor inducible promoter, an inducible promoter and a repressible promoter.

6. The cell of claim 1, wherein the regulatable promoter comprises a tet operator.

7. The cell of claim 1, further comprising a second nucleic acid comprising a second promoter operatively linked to a coding region for a reporter polypeptide, wherein the second promoter is responsive to the activated state of the GPCR.

8. The cell of claim 1, wherein the first nucleic acid comprises a second promoter operatively linked to a coding region for a reporter polypeptide, wherein the second promoter is responsive to the activated state of the GPCR.

9. The cell of claim 7, wherein the second promoter comprises a responsive element selected from the group consisting of an NFAT responsive element, a cAMP responsive element (CRE) and a kinase C-responsive promoter.

10. The cell of claim 7, wherein the reporter polypeptide is selected from the group consisting of a beta-lactamase, a fluorescent polypeptide, a luciferase, a green fluorescent protein (GFP), a chloramphenicol acetyl transferase, an alkaline phosphatase, a beta-galactosidase, an alkaline phosphatase, and a human growth hormone.

11. A method of detecting or monitoring activity of the GPCR comprising:
a. culturing the cell of claim 7 under conditions wherein the GPCR is expressed in an active state; and
b. detecting the expression of the reporter polypeptide.

12. A method of measuring the ability of a compound to affect or modulate activation of the GPCR comprising:
a. culturing the cell of claim 7 under conditions wherein the GPCR is expressed in an active state; and
b. contacting the cell with the compound; and
c. measuring expression of the reporter polypeptide.

13. The method of claim 12, further comprising a second population of the cell of step (a) in the absence of the compound and measuring expression of the reporter polypeptide in the second population of the cell.

14. The method of claim 12, wherein the measuring of the expression of the reporter polypeptide is performed before and after (b).

15. The method of claim 12, wherein the compound is determined to modulate activation of the GPCR if the measured expressions in the presence and absence of the compound differs.

16. A method for determining whether activation of a cell pathway by a first compound that activates the GPCR is capable of being modulated by a second compound comprising:
a. culturing a first population of the cell of claim 7 under conditions wherein the GPCR is expressed and contacting the first cell population with the first compound to form a first sample;
b. culturing a second population of the cell of claim 7 under conditions wherein the GPCR is expressed and contacting the second cell population with the first compound and second compound to form a second sample; and
c. measuring expression of the reporter polypeptide in the first and second samples.

17. A method of identifying a GPCR for a ligand or of identifying a ligand for a GPCR, the method comprising:
a. expressing the GPCR in the cell of claim 7;
b. contacting said cell with the ligand; and
c. detecting expression of the reporter polypeptide.

18. A method of expressing a constitutively activated GPCR from a cell comprising introducing into a population of cells a nucleic acid comprising a regulatable promoter operatively linked to a GPCR coding region and culturing the cell under conditions wherein the activated GPCR is expressed.

19. A method of constructing a stable cell line capable of expressing an activated GPCR comprising:
a. introducing into a first population of cells a nucleic acid comprising a regulatable promoter operatively linked to a GPCR coding region;
b. sorting the first population, wherein the cells have been cultured under conditions to minimize expression of the GPCR and the cells are sorted for cells that have no or low expression levels of the GPCR to create a second population of cells; and
c. sorting the second population of cells, wherein the second population of cells have been cultured under conditions to express or maximize expression of the GPCR and the cells are sorted for cells that express the GPCR in an activated state to create a third population of cells.

20. The method of claim 19, further comprising:
d. isolating clonal populations of cells from the third population of cells; and
e. characterizing the clonal populations of cells.
